(19) United States
(12) Patent Application Publication
(10) Pub. No.: US 2024/0299477 A1
(43) Pub. Date: Sep. 12, 2024

(54) COMPOSITIONS AND METHODS OF USE FOR HUMAN RHINOVIRUSES

(71) Applicant: University of Utah Research Foundation, Salt Lake City, UT (US)

(72) Inventor: Matthew W. VanBrocklin, Salt Lake City, UT (US)

(21) Appl. No.: 18/444,305

(22) Filed: Feb. 16, 2024

Related U.S. Application Data
(60) Provisional application No. 63/485,348, filed on Feb. 16, 2023.

(51) Int. Cl. A61K 35/768; A61P 35/00

(52) U.S. Cl. CPC A61K 35/768; A61P 35/00; C12N 2770/32722; C12N 2770/32732; C12N 2770/32771

(57) ABSTRACT

Disclosed are methods for treating hematologic and solid cancers in a subject, the method comprising administering locally or systemically a therapeutically effective amount of a HRVA2.s and/or HRVA45.s alone or in combination with FDA approved or experimental immunomodulatory agents such that some cells of the cancer undergo viral oncolysis, wherein the host immune system is engaged culminating in host immune activation and tumor regression in the human or animal host. Disclosed are methods for treating viral induced lesions of the skin, including HPV driven papillomas in a subject, the method comprising administering a therapeutically effective amount of HRVA2.s and/or HRVA45.s alone or in combination with immunomodulatory agents such that some cells of the papilloma cells undergo selective viral lysis, wherein the host immune system is engaged culminating in host immune activation and papilloma in the subject.

### CROSS-REFERENCE TO RELATED APPLICATIONS

This application claims the benefit of U.S. Provisional Patent Application No. 63/485,348, filed Feb. 16, 2023, which is incorporated by reference herein in its entirety.

### REFERENCE TO SEQUENCE LISTING

The Sequence Listing submitted Feb. 16, 2024 as a xml file named “21101.0451U2.xml,” created on Feb. 16, 2024, and having a size of 71,187 bytes is hereby incorporated by reference pursuant to 37 C.F.R. § 1.52(e)(5).

### BACKGROUND

Solid tumors are an abnormal mass of cells or tissue that are benign or malignant (cancerous). Solid tumors, including lung, breast, colon, skin, melanoma, glioblastoma, and pancreatic cancer account for the vast majority of cancer diagnoses and deaths each year in the United States. Despite a growing number of FDA approved drugs for treating these cancers over the past decade, including promising immunotherapeutic agents, mortality rates continue to rise.

An emerging approach in controlling cancer progression is through the use of oncolytic viruses. Oncolytic viruses are wild type or modified viruses that are able to selectively eliminate malignant cells, with limited capacity for spreading in normal host cells. Nearly all human solid cancers are susceptible to the oncolytic activity of various viruses, each possessing unique properties that mediate selective tumor lysis. Oncorine (H101), a modified adenovirus, was the first oncolytic virus approved for the treatment of cancer in the world in 2005 in China. Talimogene laherparepvec (T-VEC), a genetically modified herpes virus, was the first FDA approved oncolytic virus for the treatment of advanced melanoma in 2015. Presently there are numerous oncolytic viruses being evaluated clinically as single agents and/or in combination with FDA approved or experimental agents in nearly all solid tumor types.

Viral infection of skin keratinocytes by human papillomaviruses (HPVs) can lead to the development of plantar warts, common and genital warts that can become cancerous. Current treatments for papillomas have limited effectiveness with host immunity being the primary cause of regression. Like many other DNA viruses, HPVs disrupt key cell cycle mediators (TP53, RB) leading to unregulated cell division and papilloma development analogous to a benign cancer. In addition, like cancer HPVs impair cellular innate immunity in order to evade host immune detection and elimination.

Human Rhinoviruses (HRVs) are the leading cause of upper respiratory tract infections, such as the common cold, in humans with over 160 known serotypes. Nearly all HRV serotypes, including A45, gain entry into cells via the cellular receptor intercellular adhesion molecule-1 (ICAM-1) leading to rapid replication and cell lysis. Only 12 ‘minor group’ HRVs, including HRV A2, utilize members of the low-density lipoprotein receptor (LDLR) family as a cellular receptor. Many solid tumors and papillomas express high levels of ICAM-1 and/or LDLR including melanoma, pancreatic, and lung cancer while most normal tissue has limited or no expression of these receptors with notable exceptions such as bronchial epithelial cells (ICAM1, LDLR), endothelial cells (ICAM1), subsets of macrophages and lymphocytes (ICAM1), and adrenal gland and cortex (LDLR).

HRVs preferentially replicate at 33 C, the temperature of the upper respiratory tract, are the most common viral infection in humans and are the predominant cause of the common cold. HRVs can be transmitted via aerosols or contaminated surfaces. HRVs are lytic in nature and can replicate rapidly within hours after contact. Despite significant effort, current antiviral drugs have been largely ineffective. Numerous vaccines have been developed and have also failed as there is little, if any, cross-protection between serotypes. Each serotype is quite distinct in regards to RNA and polypeptide sequence and yet retains the ability to bind ICAM1 or LDLR. To date there are no reports of HRVs evaluated as theralytic or oncolytic viruses.

Like all Picornaviruses, HRVs are small, non-enveloped positive sense RNA viruses with receptor and host limited tropism with a rapid replication cycle. Unlike many DNA viruses (e.g., HPV) that possess elaborate means of immune evasion, sometimes lifelong, HRVs employ rapid replication and spread along with a high mutation rate to evade host immunity. Therefore, activation of host innate defense pathways are activated following HRV infection. This culminates in the release of proinflammatory factors that reverse an immunosuppressive local environment, recruit and activate adaptive host immune effectors (e.g., T cells) that culminate in the elimination of HRV infection over time. When HRVs selectively infect cancer or HPV infected cells, they retain the ability to activate innate networks leading to activation of host immune effectors that strive to eliminate HRV infected tumor or HPV infected cells. In effect, HRV infection unmasks these lesions revealing tumor and viral antigens leading to their local and systemic elimination.

### BRIEF SUMMARY

Disclosed are methods of treating cancer comprising administering to a subject in need thereof a composition comprising a human rhinovirus.

Disclosed are methods of killing cancer cells comprising administering to a cancer cell a composition comprising a human rhinovirus.

Disclosed are methods of treating hematologic and solid cancers in a subject, the method comprising administering locally or systemically a therapeutically effective amount of a HRVA2.s and/or HRVA45.s alone or in combination with FDA approved or experimental immunomodulatory agents such that some cells of the cancer undergo viral oncolysis, wherein the host immune system is engaged culminating in host immune activation and tumor regression in the human or animal host.

Disclosed are methods of treating viral induced lesions of the skin, including HPV driven papillomas in a subject, the method comprising administering a therapeutically effective amount of HRVA2.s and/or HRVA45.s alone or in combination with immunomodulatory agents such that some cells of the papilloma cells undergo selective viral lysis, wherein the host immune system is engaged culminating in host immune activation and papilloma in the subject.

Disclosed are methods of treating cancer comprising administering to a subject in need thereof one or more of the compositions disclosed herein.

Disclosed are methods of killing cancer cells comprising administering to a cancer cell one or more of the compositions disclosed herein.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1. Diagram of accrued amino acid changes in HRVA2 and HRVA45 following directed evolution/selection.

FIG. 2 shows HRV constructs.

FIG. 3 shows HRVA2.s blastn results

FIG. 4 shows HRV constructs.

FIG. 5 HaCaT cells are an immortal keratinocyte cell line derived from adult human skin. HaCaT cells were transfected with an empty vector (HaCaT), HPV1 cDNA (HaCaT HPV1), or HPV E6/E7 (HaCaT p1321). 24 hours later each cohort was infected at multiplicity of infection (MOI) as indicated (0, 0.1, 1 and 10) with HRVA2.s (left) or HRVA45.s (right) and cell death was assessed 24 hours later by CellTiter-Glo Cell Viability Assay (Promega). The percent of remaining viable cells are displayed with standard error of the mean (SEM). HaCaT cells expressing HPV1 cDNA or HPV E6/E7 genes are highly susceptible to HRV infection in a dose dependent manner while HaCaT cells not expressing HPV genes remain highly insensitive to HRV infection.

FIG. 6 demonstrates the inability of HRV to initiate tumor regression of established tumors in an immunodeficient murine xenograft model relative to untreated control cohorts.

FIGS. 7A-7C depicts the development and verification of murine tumor cells expressing human ICAM1 (A, B) and dosing schema (C) employed in a novel C57BL/6 Hu-ICAM1 immunocompetent murine tumor model.

FIG. 8 shows tumor response of established tumors following HRVA45 delivery vs vehicle control cohorts in BL6 HuICAM-1 mice. A 38% complete response (complete tumor regression) was observed in the HRVA45 cohort while no tumor regression was observed in vehicle control group. Spider plot showing tumor volume (growth) over time-treatment began when median tumor was between 100-200 cubic mm for all cohorts (Day 8).

FIG. 9 Kaplan-Meier survival curve demonstrating the survival advantage of HRVA45 treated mice relative to the vehicle control group out to 70 days in the syngeneic BL6 HuICAM-1 murine tumor model.

FIG. 10 demonstrates enhanced infiltration of key host immune effector subsets that promote tumor regression in HRVA45 cohorts relative to vehicle control or anti-PD-1 treated cohorts. CD44+hi CD62Llo represent memory/activated T cells while CD8+ KLRG1+ designate highly cytotoxic T-cells.

FIGS. 11A and 11B show that HRVs infect melanoma cells that express their cognate receptors. (A) Genomic structure of human rhinovirus. The genome is a positive-sense, single stranded RNA. Viral capsid genes VP1-VP4 are located on the 5′ end, while the non-structural components (proteases, polymerases, etc.) are on the 3′ end. (B) RT-PCR of RNA extracted from a panel of melanoma cell lines infected with HRVA2 and HRVA45. Uninfected parental lines are shown for comparison. Purified HRV stock virus was used as the positive control. The RT-PCR produces an amplicon from the 3′ end of the viral genome in the 3D^{pol}.

FIGS. 12A-12D show that HRVs induce melanoma cell death upon infection. (A,B) Assessment of cell death following HRV infection at MOI 0, 0.1, 1.0, and 10 at 24 h by flow cytometry in representative cell lines. SYTOX Green was used to stain dead cells. (C,D) Quantitation of cell death from the flow cytometry assay represented in panel. Samples were run in triplicate and analyzed using a one-way ANOVA test for statistical differences. Error bars represent the standard deviation between replicates. p values <0.05 were considered significant (p<0.05*, <0.0001****).

FIG. 13 shows that HRVs induce inflammatory signaling and adaptive immune responses in murine tumor models following delivery of HRVA2 or HRVA45. ELISA test for HRVA2 and HRVA45 antibodies in the serum of naïve and inoculated mice immediately and 2 weeks post-inoculation.

FIG. 14 demonstrates the expression of human GAPDH control (left) ICAM1 (right) in indicated organs in wt C57BL/6 (−) mice vs newly generated transgenic C57BL/6 HuICAM1 mice (+).

FIGS. 15A-15C demonstrate the restriction of HRVA45 to infecting cells expressing human ICAM1 (A) Expression of human ICAM1 in melanoma cell lines. (+) is murine YUMM2.1 expressing human ICAM1, (−) is YUMM2.1 cells, C3 is a murine melanocyte cell line, the remainder are human melanoma cell lines. (B) Detection of HRV-A45 replication by RT-PCR in murine melanoma cell lines either mock infected or infected with HRV-A45 (red+). Bolded cell lines indicate constitutive expression of human ICAM1. (C) Infection of human melanoma cell lines by HRV-A45 detected at 24 hours by RT-PCR. Red+ indicates infection with HRV-A45 at an MOI of 1. Bands of 290 bp are indicative of viral infection.

FIGS. 16A-16C show HRV-A45 infection of human breast cancer and glioma cell lines. (A) Expression of ICAM1 in human breast cancer and glioma cell lines. (B) Detection of HRV-A45 replication by RT-PCR in breast cancer cell lines 24 hours post infection. Red+ indicates HRV-A45 infection at MOI=1. (C) Detection of active HRV-45 infection (MOI=1) in human glioma cell lines by RT-PCR. Bands of 290 bp are indicative of viral infection.

FIG. 17 shows limited capacity of HRVA2 and HRVA45 to infect canine tumor cell lines at 48 hours. Cell death as determined by SYTOX green staining and FACS analysis. HELA-H1 cells are a positive control for each virus. CHL-1 cells lack detectable ICAM1 expression. Two MOIs were used for each virus as indicated (0.5, 5). The first five cell lines are canine tumor cells.

FIG. 18 shows HRV oncolysis of a broad spectrum of human cancer types at 48 hours. Cell death was determined by SYTOX green staining by FACS 48 hours following HRV-A2 or HRV-A45 infection. Following one hour infection with each virus at an MOI of 1, cells were washed and fresh growth medium added. HELA-H1 cells are a positive control.

FIG. 19 shows HRV infection of human cancer cell leads to enhanced cytokine production. Each cell line indicated was infected with HRV A2 at an MOI of 1 and 25 uL supernatant was collected at 24 hours. Uninfected cells have the cell line name only while HRV infected cells are indicated with HRV A2. Each soluble factor is expressed as pg/mL. Cytokine/Chemokine/Growth Factor 45-Plex Human ProcartaPlex™ Panel 1 EPX450-12171-901 was used and run on a MAGPIX. The fold change is displayed in the upper right hand corner of each plot.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Headings are provided for convenience only and are not to be construed to limit the invention in any manner. Embodiments illustrated under any heading or in any portion of the disclosure may be combined with embodiments illustrated under the same or any other heading or other portion of the disclosure.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a rhinovirus” includes a plurality of such rhinoviruses, reference to “the composition” is a reference to one or more compositions and equivalents thereof known to those skilled in the art, and so forth.

By “treat” is meant to administer a virus, composition, or nucleic acid of the invention to a subject, such as a human or other mammal (for example, an animal model), that has an increased susceptibility for developing a disease, disorder or infection in order to prevent or delay onset of the disease disorder or infection, prevent or delay a worsening of the effects of the disease, disorder or infection, or to partially or fully reverse the effects of the disease, disorder or infection. In some aspects, treat can mean to ameliorate a symptom of a disease, disorder or infection.

By “prevent” is meant to minimize the chance that a subject who has an increased susceptibility for developing a disease, disorder or infection will actually develop the disease, disorder or infection.

As used herein, the term “subject” or “patient” can be used interchangeably and refer to any organism to which a virus or composition of the invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates, and the like.

As used herein, the terms “administering” and “administration” refer to any method of providing a disclosed virus or composition of the invention to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration for a disclosed composition or a disclosed exosome so as to treat a subject.

The terms “variant” and “mutant” are used interchangeably herein. As used herein, the term “mutant” refers to a modified nucleic acid or protein which displays the same characteristics when compared to a reference nucleic acid or protein sequence. A variant can be at least 65, 70, 75, 80, 85, 90, 95, or 99 percent homologous to a reference sequence. In some aspects, a reference sequence can be a fragment of one or more of the disclosed HRV sequences. A “variant” can mean a difference in some way from the reference sequence other than just a simple deletion of an N- and/or C-terminal amino acid. A variant can also be a difference in the nucleotide sequence. Variants can also or alternatively include at least one substitution and/or at least one addition; there may also be at least one deletion. Alternatively or in addition, variants can comprise modifications, such as non-natural residues at one or more positions with respect to a reference nucleic acid or protein.

Substitutions, deletions, insertions or any combination thereof may be used to arrive at a final derivative or variant. Generally, these changes are done on a few nucleotides to minimize the alteration of the molecule. However, larger changes may be tolerated in certain circumstances.

Generally, the amino acid or nucleotide identity between individual variant sequences can be at least 90%, 91%, 92%, 93%, 94%, 95%, 96%, 97%, 98%, 99%, or 100%. Thus, a “variant sequence” can be one with the specified identity to the parent or reference sequence (e.g. wild-type sequence) of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. For example, a “variant sequence” can be a sequence that contains 1, 2, or 3 4 nucleotide base changes as compared to the parent or reference sequence of the invention, and shares or improves biological function, specificity and/or activity of the parent sequence. Thus, a “variant sequence” can be one with the specified identity to the parent sequence of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. The variant sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of a reference sequence (e.g. wild-type sequence).

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

The term “percent (%) homology” is used interchangeably herein with the term “percent (%) identity” and refers to the level of nucleic acid or amino acid sequence identity when aligned with a wild type sequence using a sequence alignment program. For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 80, 85, 90, 95, 98% or more sequence identity to a given sequence, e.g., the coding sequence for anyone of the inventive polypeptides, as described herein. Exemplary computer programs which can be used to determine identity between two sequences include, but are not limited to, the suite of BLAST programs, e.g., BLASTN, BLASTX, and TBLASTX, BLASTP and TBLASTN, publicly available on the Internet. Sec also, Altschul, et al., 1990 and Altschul, et al., 1997. Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62 matrix. (See, e.g., Altschul, S. F., et al., Nucleic Acids Res. 25:3389-3402, 1997.) A preferred alignment of selected sequences in order to determine“% identity” between two or more sequences, is performed using for example, the CLUSTAL-W program in Mac Vector version 13.0.7, operated with default parameters, including an open gap penalty of 10.0, an extended gap penalty of 0.1, and a BLOSUM 30 similarity matrix.

As used herein, the term “therapeutically effective amount” of a composition (e.g. human rhinovirus) as provided herein is meant a sufficient amount of the composition to provide the desired therapeutic effect (e.g. killing of cancer cells or inducing immune response). The exact amount required will vary from subject to subject, depending on the species, age, and general condition of the subject, the severity of disease (or underlying genetic defect) that is being treated, the particular composition used, its mode of administration, and the like. Thus, it is not possible to specify an exact “therapeutically effective amount.” However, an appropriate “therapeutically effective amount” may be determined by one of ordinary skill in the art using only routine experimentation.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Rhinoviruses

Disclosed are variant rhinoviruses compared to wild type rhinovirus. In some aspects, wild type rhinovirus is the synthetic wild-type reference GenBank: X02316.1 or FJ445132.1.

In some aspects, the rhinovirus can be any of those described in Table 1 or Table 2.

In some aspects, the rhinovirus can be a variant of any of those described in Table 1 or Table 2 as long as they at least contain the mutation shown in Tables 1 and/or 2. For example, each of the disclosed variant rhinoviruses can have one or more mutations. In some aspects, disclosed are rhinoviruses comprising at least one mutation of Table 1 or Table 2.

In some aspects, a rhinovirus can be encoded by the nucleic acid sequence of SEQ ID NO:1, SEQ ID NO:3, or a variant thereof. In some aspects, a rhinovirus can comprise the amino acid sequence of SEQ ID NO:2, SEQ ID NO:4, or a variant thereof. In some aspects, a rhinovirus can be encoded by a nucleic acid sequence 70, 75, 80, 85, 90, 95, or 99% identical to SEQ ID NO: 1 or SEQ ID NO:3. In some aspects, a rhinovirus can comprise an amino acid sequence 70, 75, 80, 85, 90, 95, or 99% identical to of SEQ ID NO:2 or SEQ ID NO:4.

Disclosed are human rhinoviruses comprising one or more single nucleotide polymorphisms (SNPs), wherein the SNPs are in the nucleic acid sequences in the 5′UTR, VP4, VP3, VP1, P2-C polypeptide, P3-A polypeptide. VpG protein. Protease 3C, or RDRP regions of the rhinovirus when compared to the nucleic acid sequence that is available under the GenBank accession number X02316.1. In some aspects, the one or SNPs are at positions 228, 491, 740, 744, 1735, 2491, 2517, 2714, 4755, 4756, 4854, 5018, 5023, 5026, 5027, 5152, 5300, 5521, or 6238 when compared to the nucleic acid sequence that is available under the GenBank accession number X02316.1. In some aspects, the one or SNPs are A228U, U491C, C740A, A744G, A1735G, A2491U, G2517A, G2714A, GC4755/4756CG, A4854G, G5018A, G5023A, GA5026/5027AG, G5152A, A5300G, C5521U, or A6238G when compared to the nucleic acid sequence that is available under the GenBank accession number X02316.1. In some aspects, the human rhinovirus comprises SNPs at positions 228, 491, 740, 744, 1735, 2491, 2517, 2714, 4755, 4756, 4854, 5018, 5023, 5026, 5027, 5152, 5300, 5521, or 6238 when compared to the nucleic acid sequence that is available under the GenBank accession number X02316.1. In some aspects, the human rhinovirus comprises the SNPs A228U, U491C, C740A, A744G, A1735G, A2491U, G2517A, G2714A, GC4755/4756CG, A4854G, G5018A, G5023A, GA5026/5027AG, G5152A, A5300G, C5521U, and A6238G when compared to the nucleic acid sequence that is available under the GenBank accession number X02316.1.

Disclosed are human rhinoviruses comprising one or more single nucleotide polymorphisms (SNP), wherein the SNPs are in the nucleic acid sequences in the VP3, VP1, P2-B polypeptide, P2-C polypeptide, or P3-A polypeptide regions when compared to the nucleic acid sequence that is available under the GenBank accession number FJ445132.1. In some aspects, the one or more SNPs are at positions 1665, 1746, 2953, 3262, 3631, 3817, 4007, 4020, 4194, 4664, 4695, 4917, or 4952 when compared to the nucleic acid sequence that is available under the GenBank accession number FJ445132.1. In some aspects, the one or more SNPs are A1665U, A1746G, G2953C, U3262C, G3631A, A3817G, A4007G, U4020C, A4194G, A4664G, U4695C, A4917G, or U4952C when compared to the nucleic acid sequence that is available under the GenBank accession number FJ445132.1. In some aspects, the human rhinovirus comprises SNPs at positions 1665, 1746, 2953, 3262, 3631, 3817, 4007, 4020, 4194, 4664, 4695, 4917, and 4952 when compared to the nucleic acid sequence that is available under the GenBank accession number FJ445132.1. In some aspects, the human rhinovirus comprises the SNPs A1665U, A1746G, G2953C, U3262C, G3631A, A3817G, A4007G, U4020C, A4194G, A4664G, U4695C, A4917G, and U4952C when compared to the nucleic acid sequence that is available under the GenBank accession number FJ445132.1.

In some aspects, the disclosed rhinoviruses can comprise or be encoded by any of the nucleic acid sequences disclosed herein. For example, the disclosed rhinoviruses can comprise a nucleic acid of SEQ ID NO:1, 2, or variant thereof and/or can be encoded by a nucleic acid of SEQ ID NO:1, 2, or variant thereof.

### C. Compositions

Disclosed are compositions comprising at least one or more of the disclosed rhinoviruses. For example, disclosed are compositions comprising one or more rhinoviruses comprising one or more SNPs of Table 1 or Table 2. Disclosed are compositions comprising a rhinovirus encoded by the nucleic acid sequence of SEQ ID NO:1, SEQ ID NO:3, or a variant thereof. In some aspects, disclosed are compositions comprising a rhinovirus comprising the amino acid sequence of SEQ ID NO:2, SEQ ID NO:4, or a variant thereof.

In some instances, the compositions can further comprise a pharmaceutically acceptable carrier. Thus, also disclosed are pharmaceutical compositions. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, P A 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Other formulations for administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

The disclosed rhinoviruses and/or compositions can be formulated and/or administered in or with a pharmaceutically acceptable carrier. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug (e.g. peptide) in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Thus, the compositions disclosed herein can comprise lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subject's lung cells. Regarding liposomes, see, e.g., Brigham et al. Am. J. Resp. Cell. Mol. Biol. 1:95 100 (1989); Felgner et al. Proc. Natl. Acad. Sci USA 84:7413 7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In some instances, disclosed are pharmaceutical compositions comprising any of the disclosed peptides described herein, or a pharmaceutically acceptable salt or solvate thereof, and a pharmaceutically acceptable carrier, buffer, or diluent. In various aspects, the peptide of the pharmaceutical composition is encapsulated in a delivery vehicle. In a further aspect, the delivery vehicle is a liposome, a microcapsule, or a nanoparticle. In a still further aspect, the delivery vehicle is PEG-ylated.

In the methods described herein, delivery of the compositions to cells can be via a variety of mechanisms. As defined above, disclosed herein are compositions comprising any one or more of the peptides described herein and can also include a carrier such as a pharmaceutically acceptable carrier. For example, disclosed are pharmaceutical compositions, comprising the peptides disclosed herein, and a pharmaceutically acceptable carrier. In one aspect, disclosed are pharmaceutical compositions comprising the disclosed peptides. That is, a pharmaceutical composition can be provided comprising a therapeutically effective amount of at least one disclosed peptide or at least one product of a disclosed method and a pharmaceutically acceptable carrier.

In certain aspects, the disclosed pharmaceutical compositions comprise the disclosed peptides (including pharmaceutically acceptable salt(s) thereof) as an active ingredient, a pharmaceutically acceptable carrier, and, optionally, other therapeutic ingredients or adjuvants. The instant compositions include those suitable for nasal, oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In practice, the peptides described herein, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

By “pharmaceutically acceptable” is meant a material or carrier that would be selected to minimize any degradation of the active ingredient and to minimize any adverse side effects in the subject, as would be well known to one of skill in the art. The peptides described herein, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. Other examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, P A 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

In order to enhance the solubility and/or the stability of the disclosed peptides in pharmaceutical compositions, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also, co-solvents such as alcohols may improve the solubility and/or the stability of the compounds according to the invention in pharmaceutical compositions.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Because of the case in administration, oral administration can be used, and tablets and capsules represent the most advantageous oral dosage unit forms in which case solid pharmaceutical carriers are obviously employed. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their case of administration, tablets and capsules are the preferred oral dosage units whereby solid pharmaceutical carriers are employed. Optionally, tablets can be coated by standard aqueous or nonaqueous techniques.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

A tablet containing the compositions of the present invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a disclosed peptide (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. Typically, the final injectable form should be sterile and should be effectively fluid for easy syringability. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. Also included are solid form preparations that are intended to be converted, shortly before use, to liquid form preparations.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot on, as an ointment.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Formulations for optical administration may include ointments, lotions, creams, gels, drops, suppositories, sprays, liquids and powders. Conventional pharmaceutical carriers, aqueous, powder or oily bases, thickeners and the like may be desirable.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a disclosed peptide, and/or pharmaceutically acceptable salts thereof, can also be prepared in powder or liquid concentrate form.

The exact dosage and frequency of administration depends on the particular disclosed peptide, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compositions.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

### D. Methods

Disclosed are methods of using the disclosed rhinoviruses or compositions.

### 1. Methods of Treating Cancer

Disclosed are methods of treating cancer in a subject having cancer comprising administering to the subject one or more of the disclosed human rhinoviruses or one or more of the disclosed compositions comprising a human rhinovirus. In some aspects, the human rhinovirus is one or more of the variant human rhinoviruses disclosed herein.

In some aspects, the cancer can be a solid tumor. For example, in some aspects, the cancer can be head and neck cancer, skin cancer, breast cancer, or any HPV associated cancer. In some aspects, the cancer is multiple myeloma.

In some aspects, administering can be local or systemic administration.

In some aspects, the one or more human rhinoviruses, administered alone or as a composition, comprises one or more single nucleotide polymorphisms (SNPs), wherein the SNPs are in the nucleic acid sequences in the 5′UTR, VP4, VP3, VP1, P2-C polypeptide, P3-A polypeptide. VpG protein. Protease 3C, or RDRP regions of the rhinovirus when compared to the nucleic acid sequence that is available under the GenBank accession number X02316.1. In some aspects, the one or SNPs are at positions 228, 491, 740, 744, 1735, 2491, 2517, 2714, 4755, 4756, 4854, 5018, 5023, 5026, 5027, 5152, 5300, 5521, or 6238 when compared to the nucleic acid sequence that is available under the GenBank accession number X02316.1. In some aspects, the one or SNPs are A228U, U491C, C740A, A744G, A1735G, A2491U, G2517A, G2714A, GC4755/4756CG, A4854G, G5018A, G5023A, GA5026/5027AG, G5152A, A5300G, C5521U, or A6238G when compared to the nucleic acid sequence that is available under the GenBank accession number X02316.1. In some aspects, the human rhinovirus comprises SNPs at positions 228, 491, 740, 744, 1735, 2491, 2517, 2714, 4755, 4756, 4854, 5018, 5023, 5026, 5027, 5152, 5300, 5521, or 6238 when compared to the nucleic acid sequence that is available under the GenBank accession number X02316.1. In some aspects, the human rhinovirus comprises the SNPs A228U, U491C, C740A, A744G, A1735G, A2491U, G2517A, G2714A, GC4755/4756CG, A4854G, G5018A, G5023A, GA5026/5027AG, G5152A, A5300G, C5521U, and A6238G when compared to the nucleic acid sequence that is available under the GenBank accession number X02316.1.

In some aspects, the one or more human rhinoviruses, administered alone or as a composition, comprises one or more single nucleotide polymorphisms (SNP), wherein the SNPs are in the nucleic acid sequences in the VP3, VP1, P2-B polypeptide, P2-C polypeptide, or P3-A polypeptide regions when compared to the nucleic acid sequence that is available under the GenBank accession number FJ445132.1. In some aspects, the one or more SNPs are at positions 1665, 1746, 2953, 3262, 3631, 3817, 4007, 4020, 4194, 4664, 4695, 4917, or 4952 when compared to the nucleic acid sequence that is available under the GenBank accession number FJ445132.1. In some aspects, the one or more SNPs are A1665U, A1746G, G2953C, U3262C, G3631A, A3817G, A4007G, U4020C, A4194G, A4664G, U4695C, A4917G, or U4952C when compared to the nucleic acid sequence that is available under the GenBank accession number FJ445132.1. In some aspects, the human rhinovirus comprises SNPs at positions 1665, 1746, 2953, 3262, 3631, 3817, 4007, 4020, 4194, 4664, 4695, 4917, and 4952 when compared to the nucleic acid sequence that is available under the GenBank accession number FJ445132.1. In some aspects, the human rhinovirus comprises the SNPs A1665U, A1746G, G2953C, U3262C, G3631A, A3817G, A4007G, U4020C, A4194G, A4664G, U4695C, A4917G, and U4952C when compared to the nucleic acid sequence that is available under the GenBank accession number FJ445132.1.

In some aspects, the rhinovirus can be encoded by the nucleic acid sequence of SEQ ID NO:1, SEQ ID NO:3, or a variant thereof. In some aspects, a rhinovirus can comprise the amino acid sequence of SEQ ID NO:2, SEQ ID NO:4, or a variant thereof. In some aspects, a rhinovirus can be encoded by a nucleic acid sequence 70, 75, 80, 85, 90, 95, or 99% identical to SEQ ID NO: 1 or SEQ ID NO:3. In some aspects, a rhinovirus can comprise an amino acid sequence 70, 75, 80, 85, 90, 95, or 99% identical to of SEQ ID NO:2 or SEQ ID NO:4.

In some aspects, a combination of rhinoviruses can be used to treat a subject having cancer. In some aspects, two or more rhinoviruses can be present in a single composition. In some aspects, two separate compositions, each comprising a different rhinovirus, can be administered for the treatment of cancer. For example, a combination of HRVA2 and HRVA45 can be used in the disclosed methods. In some aspects, each of the HRVA2 and HRVA45 are variants of wildtype viruses identified by GenBank references X02316.1 or FJ445132.1. In some aspects, the combination of HRVA2 and HRVA45 can be any of those disclosed herein.

Also disclosed are methods of treating cancer in a subject having cancer comprising administering to the subject one or more of the disclosed human rhinoviruses or one or more of the disclosed compositions comprising a human rhinovirus and further comprising administering a known anti-cancer therapeutic to the subject having cancer. In some aspects, the known anti-cancer therapeutic is an immune checkpoint inhibitor. In some aspects, an immune checkpoint inhibitor can be a CTLA-4 inhibitor, PD-1 inhibitor or PD-L1 inhibitor.

Disclosed are methods for treating hematologic and solid cancers in a subject, the method comprising administering locally or systemically a therapeutically effective amount of a HRVA2.s and/or HRVA45.s alone or in combination with FDA approved or experimental immunomodulatory agents such that some cells of the cancer undergo viral oncolysis, wherein the host immune system is engaged culminating in host immune activation and tumor regression in the subject.

Disclosed are methods of treating a hematologic or a solid cancers in a subject, the method comprising administering locally or systemically a therapeutically effective amount of one or more of the compositions disclosed herein alone or in combination with FDA approved or experimental immunomodulatory agents such that some cells of the cancer undergo viral oncolysis, wherein the host immune system is engaged culminating in host immune activation and tumor regression in the human or animal host.

In some aspects, the human rhinovirus induces tumor regression, thus treating cancer in a subject.

In some aspects, the human rhinovirus induces pro-inflammatory cytokines. In some aspects, pro-inflammatory cytokines can regulate growth, cell activation, differentiation, and homing of immune cells to the sites of cancer cells with the aim to control and eradicate the cancer cells.

In some aspects, the cancer cell expresses ICAM-1 and/or LDLR on the surface. HRVA2, and variants thereof, bind to LDLR on the surface of cancer cells. HRVA45, and variants thereof, bind to ICAM1 on the surface of cancer cells. In some aspects, ICAM-1 and/or LDLR can first be upregulated in a cancer cell in a subject prior to administering the human rhinovirus or composition comprising a human rhinovirus.

### 2. Methods of Killing Cancer Cells

Disclosed are methods of killing a cancer cell comprising contacting a cancer cell with a human rhinovirus or a composition comprising a human rhinovirus. In some aspects, contacting a cancer cell can include exposing a cancer cell to a composition comprising a human rhinovirus or administering a composition comprising a human rhinovirus to a cancer cell or a container comprising a cancer cell.

In some aspects, the human rhinovirus induces an immune response, thereby killing cancer cells. In some aspects, the human rhinovirus increases proinflammatory response, thereby killing cancer cells.

In some aspects, the cancer can be a solid tumor. For example, in some aspects, the cancer can be head and neck cancer, skin cancer, breast cancer, or any HPV associated cancer. In some aspects, the cancer is multiple myeloma.

In some aspects, the cancer cell is in a subject. If the cancer cell is in a subject, the method of killing a cancer cell comprises administering a human rhinovirus or a composition comprising a human rhinovirus to a subject having cancer. In some aspects, the cancer cell is in vitro. The in vitro methods comprise contact a cancer cell with a human rhinovirus or a composition comprising a human rhinovirus or exposing a cancer cell to a human rhinovirus or a composition comprising a human rhinovirus. In some aspects, the in vitro method comprises administering a human rhinovirus or a composition comprising a human rhinovirus to a container (e.g. culture well or plate) comprising cancer cells.

In some aspects, the one or more human rhinoviruses, contacted to a cancer cell alone or as a composition, comprises one or more single nucleotide polymorphisms (SNPs), wherein the SNPs are in the nucleic acid sequences in the 5′UTR, VP4, VP3, VP1, P2-C polypeptide, P3-A polypeptide. VpG protein. Protease 3C, or RDRP regions of the rhinovirus when compared to the nucleic acid sequence that is available under the GenBank accession number X02316.1. In some aspects, the one or SNPs are at positions 228, 491, 740, 744, 1735, 2491, 2517, 2714, 4755, 4756, 4854, 5018, 5023, 5026, 5027, 5152, 5300, 5521, or 6238 when compared to the nucleic acid sequence that is available under the GenBank accession number X02316.1. In some aspects, the one or SNPs are A228U, U491C, C740A, A744G, A1735G, A2491U, G2517A, G2714A, GC4755/4756CG, A4854G, G5018A, G5023A, GA5026/5027AG, G5152A, A5300G, C5521U, or A6238G when compared to the nucleic acid sequence that is available under the GenBank accession number X02316.1. In some aspects, the human rhinovirus comprises SNPs at positions 228, 491, 740, 744, 1735, 2491, 2517, 2714, 4755, 4756, 4854, 5018, 5023, 5026, 5027, 5152, 5300, 5521, or 6238 when compared to the nucleic acid sequence that is available under the GenBank accession number X02316.1. In some aspects, the human rhinovirus comprises the SNPs A228U, U491C, C740A, A744G, A1735G, A2491U, G2517A, G2714A, GC4755/4756CG, A4854G, G5018A, G5023A, GA5026/5027AG, G5152A, A5300G, C5521U, and A6238G when compared to the nucleic acid sequence that is available under the GenBank accession number X02316.1.

In some aspects, the one or more human rhinoviruses, contacted to a cancer cell alone or as a composition, comprises one or more single nucleotide polymorphisms (SNP), wherein the SNPs are in the nucleic acid sequences in the VP3, VP1, P2-B polypeptide, P2-C polypeptide, or P3-A polypeptide regions when compared to the nucleic acid sequence that is available under the GenBank accession number FJ445132.1. In some aspects, the one or more SNPs are at positions 1665, 1746, 2953, 3262, 3631, 3817, 4007, 4020, 4194, 4664, 4695, 4917, or 4952 when compared to the nucleic acid sequence that is available under the GenBank accession number FJ445132.1. In some aspects, the one or more SNPs are A1665U, A1746G, G2953C, U3262C, G3631A, A3817G, A4007G, U4020C, A4194G, A4664G, U4695C, A4917G, or U4952C when compared to the nucleic acid sequence that is available under the GenBank accession number FJ445132.1. In some aspects, the human rhinovirus comprises SNPs at positions 1665, 1746, 2953, 3262, 3631, 3817, 4007, 4020, 4194, 4664, 4695, 4917, and 4952 when compared to the nucleic acid sequence that is available under the GenBank accession number FJ445132.1. In some aspects, the human rhinovirus comprises the SNPs A1665U, A1746G, G2953C, U3262C, G3631A, A3817G, A4007G, U4020C, A4194G, A4664G, U4695C, A4917G, and U4952C when compared to the nucleic acid sequence that is available under the GenBank accession number FJ445132.1.

In some aspects, the rhinovirus can be encoded by the nucleic acid sequence of SEQ ID NO:1, SEQ ID NO:3, or a variant thereof. In some aspects, a rhinovirus can comprise the amino acid sequence of SEQ ID NO:2, SEQ ID NO:4, or a variant thereof. In some aspects, a rhinovirus can be encoded by a nucleic acid sequence 70, 75, 80, 85, 90, 95, or 99% identical to SEQ ID NO: 1 or SEQ ID NO:3. In some aspects, a rhinovirus can comprise an amino acid sequence 70, 75, 80, 85, 90, 95, or 99% identical to of SEQ ID NO:2 or SEQ ID NO:4.

In some aspects, a combination of rhinoviruses can be used to kill cancer cells. In some aspects, two or more rhinoviruses can be present in a single composition. In some aspects, two separate compositions, each comprising a different rhinovirus, can be contacted to a cancer cell for killing a cancer cell. For example, a combination of HRVA2 and HRVA45 can be used in the disclosed methods. In some aspects, each of the HRVA2 and HRVA45 are variants of wildtype viruses identified by GenBank references X02316.1 or FJ445132.1. In some aspects, the combination of HRVA2 and HRVA45 can be any of those disclosed herein.

In some aspects, the cancer cell expresses ICAM-1 and/or LDLR on the surface. HRVA2, and variants thereof, bind to LDLR on the surface of cancer cells. HRVA45, and variants thereof, bind to ICAM1 on the surface of cancer cells. In some aspects, ICAM-1 and/or LDLR can first be upregulated in a cancer cell in a subject prior to administering the human rhinovirus or composition comprising a human rhinovirus.

### 3. Methods of Inducing Pro-Inflammatory Cytokines in a Cancer Cell

Disclosed are methods of inducing pro-inflammatory cytokines in a cancer cell comprising contacting the cancer cell with a composition comprising a human rhinovirus. IN some aspects, both cancer cells and non-cancer cells (e.g. immune cells) can produce cytokines in response to the human rhinovirus. In some aspects, the pro-inflammatory cytokines can be, but are not limited to, IL-6, CXCL10, CCL5, IFN-α, IL-1, IL-8, MIP1A, and/or MIP1B.

In some aspects, the cancer cell can be from a solid tumor. For example, in some aspects, the cancer cell can be from head and neck cancer, skin cancer, breast cancer, or any HPV associated cancer. In some aspects, the cancer cell is a multiple myeloma cell.

In some aspects, the cancer cell is in a subject. If the cancer cell is in a subject, the method of inducing pro-inflammatory cytokines in a cancer cell in a subject comprises administering a human rhinovirus or a composition comprising a human rhinovirus to a subject having cancer. In some aspects, the cancer cell is in vitro. The in vitro methods comprise contacting a cancer cell with a human rhinovirus or a composition comprising a human rhinovirus or exposing a cancer cell to a human rhinovirus or a composition comprising a human rhinovirus. In some aspects, the in vitro method comprises administering a human rhinovirus or a composition comprising a human rhinovirus to a container (e.g. culture well or plate) comprising cancer cells.

In some aspects, the one or more human rhinoviruses, contacted to a cancer cell alone or as a composition, comprises one or more single nucleotide polymorphisms (SNPs), wherein the SNPs are in the nucleic acid sequences in the 5′UTR, VP4, VP3, VP1, P2-C polypeptide, P3-A polypeptide. VpG protein. Protease 3C, or RDRP regions of the rhinovirus when compared to the nucleic acid sequence that is available under the GenBank accession number X02316.1. In some aspects, the one or SNPs are at positions 228, 491, 740, 744, 1735, 2491, 2517, 2714, 4755, 4756, 4854, 5018, 5023, 5026, 5027, 5152, 5300, 5521, or 6238 when compared to the nucleic acid sequence that is available under the GenBank accession number X02316.1. In some aspects, the one or SNPs are A228U, U491C, C740A, A744G, A1735G, A2491U, G2517A, G2714A, GC4755/4756CG, A4854G, G5018A, G5023A, GA5026/5027AG, G5152A, A5300G, C5521U, or A6238G when compared to the nucleic acid sequence that is available under the GenBank accession number X02316.1. In some aspects, the human rhinovirus comprises SNPs at positions 228, 491, 740, 744, 1735, 2491, 2517, 2714, 4755, 4756, 4854, 5018, 5023, 5026, 5027, 5152, 5300, 5521, or 6238 when compared to the nucleic acid sequence that is available under the GenBank accession number X02316.1. In some aspects, the human rhinovirus comprises the SNPs A228U, U491C, C740A, A744G, A1735G, A2491U, G2517A, G2714A, GC4755/4756CG, A4854G, G5018A, G5023A, GA5026/5027AG, G5152A, A5300G, C5521U, and A6238G when compared to the nucleic acid sequence that is available under the GenBank accession number X02316.1.

In some aspects, the one or more human rhinoviruses, contacted to a cancer cell alone or as a composition, comprises one or more single nucleotide polymorphisms (SNP), wherein the SNPs are in the nucleic acid sequences in the VP3, VP1, P2-B polypeptide, P2-C polypeptide, or P3-A polypeptide regions when compared to the nucleic acid sequence that is available under the GenBank accession number FJ445132.1. In some aspects, the one or more SNPs are at positions 1665, 1746, 2953, 3262, 3631, 3817, 4007, 4020, 4194, 4664, 4695, 4917, or 4952 when compared to the nucleic acid sequence that is available under the GenBank accession number FJ445132.1. In some aspects, the one or more SNPs are A1665U, A1746G, G2953C, U3262C, G3631A, A3817G, A4007G, U4020C, A4194G, A4664G, U4695C, A4917G, or U4952C when compared to the nucleic acid sequence that is available under the GenBank accession number FJ445132.1. In some aspects, the human rhinovirus comprises SNPs at positions 1665, 1746, 2953, 3262, 3631, 3817, 4007, 4020, 4194, 4664, 4695, 4917, and 4952 when compared to the nucleic acid sequence that is available under the GenBank accession number FJ445132.1. In some aspects, the human rhinovirus comprises the SNPs A1665U, A1746G, G2953C, U3262C, G3631A, A3817G, A4007G, U4020C, A4194G, A4664G, U4695C, A4917G, and U4952C when compared to the nucleic acid sequence that is available under the GenBank accession number FJ445132.1.

In some aspects, the rhinovirus can be encoded by the nucleic acid sequence of SEQ ID NO:1, SEQ ID NO:3, or a variant thereof. In some aspects, a rhinovirus can comprise the amino acid sequence of SEQ ID NO:2, SEQ ID NO:4, or a variant thereof. In some aspects, a rhinovirus can be encoded by a nucleic acid sequence 70, 75, 80, 85, 90, 95, or 99% identical to SEQ ID NO:1 or SEQ ID NO:3. In some aspects, a rhinovirus can comprise an amino acid sequence 70, 75, 80, 85, 90, 95, or 99% identical to of SEQ ID NO:2 or SEQ ID NO:4.

In some aspects, a combination of rhinoviruses can be used to induce pro-flammatory cytokines. In some aspects, two or more rhinoviruses can be present in a single composition. In some aspects, two separate compositions, each comprising a different rhinovirus, can be contacted to a cancer cell for inducing pro-flammatory cytokines. For example, a combination of HRVA2 and HRVA45 can be used in the disclosed methods. In some aspects, each of the HRVA2 and HRVA45 are variants of wildtype viruses identified by GenBank references X02316.1 or FJ445132.1. In some aspects, the combination of HRVA2 and HRVA45 can be any of those disclosed herein.

In some aspects, the cancer cell expresses ICAM-1 and/or LDLR on the surface. HRVA2, and variants thereof, bind to LDLR on the surface of cancer cells. HRVA45, and variants thereof, bind to ICAM1 on the surface of cancer cells. In some aspects, ICAM-1 and/or LDLR can first be upregulated in a cancer cell in a subject prior to administering the human rhinovirus or composition comprising a human rhinovirus.

In some aspects, the human rhinovirus induces pro-inflammatory cytokines. In some aspects, pro-inflammatory cytokines can regulate growth, cell activation, differentiation, and homing of immune cells to the sites of cancer cells with the aim to control and eradicate the cancer cells.

### 4. Methods of Inducing Tumor Regression

Disclosed are methods of inducing tumor regression in a subject having cancer comprising administering to the subject having cancer a composition comprising a human rhinovirus. In some aspects, the tumor regression is immune response dependent.

In some aspects, the cancer can be a solid tumor. For example, in some aspects, the cancer can be head and neck cancer, skin cancer, breast cancer, or any HPV associated cancer. In some aspects, the cancer is multiple myeloma.

In some aspects, tumor regression can be at least a 1, 2, 3, 4, 5, 6, 7, 8, 9, 10, 20, 30, 40, or 50% decrease in tumor size compared to before administering the human rhinovirus.

In some aspects, administering can be local or systemic administration.

In some aspects, the one or more human rhinoviruses, administered alone or as a composition, comprises one or more single nucleotide polymorphisms (SNPs), wherein the SNPs are in the nucleic acid sequences in the 5′UTR, VP4, VP3, VP1, P2-C polypeptide, P3-A polypeptide. VpG protein. Protease 3C, or RDRP regions of the rhinovirus when compared to the nucleic acid sequence that is available under the GenBank accession number X02316.1. In some aspects, the one or SNPs are at positions 228, 491, 740, 744, 1735, 2491, 2517, 2714, 4755, 4756, 4854, 5018, 5023, 5026, 5027, 5152, 5300, 5521, or 6238 when compared to the nucleic acid sequence that is available under the GenBank accession number X02316.1. In some aspects, the one or SNPs are A228U, U491C, C740A, A744G, A1735G, A2491U, G2517A, G2714A, GC4755/4756CG, A4854G, G5018A, G5023A, GA5026/5027AG, G5152A, A5300G, C5521U, or A6238G when compared to the nucleic acid sequence that is available under the GenBank accession number X02316.1. In some aspects, the human rhinovirus comprises SNPs at positions 228, 491, 740, 744, 1735, 2491, 2517, 2714, 4755, 4756, 4854, 5018, 5023, 5026, 5027, 5152, 5300, 5521, or 6238 when compared to the nucleic acid sequence that is available under the GenBank accession number X02316.1. In some aspects, the human rhinovirus comprises the SNPs A228U, U491C, C740A, A744G, A1735G, A2491U, G2517A, G2714A, GC4755/4756CG, A4854G, G5018A, G5023A, GA5026/5027AG, G5152A, A5300G, C5521U, and A6238G when compared to the nucleic acid sequence that is available under the GenBank accession number X02316.1.

In some aspects, the one or more human rhinoviruses, administered alone or as a composition, comprises one or more single nucleotide polymorphisms (SNP), wherein the SNPs are in the nucleic acid sequences in the VP3, VP1, P2-B polypeptide, P2-C polypeptide, or P3-A polypeptide regions when compared to the nucleic acid sequence that is available under the GenBank accession number FJ445132.1. In some aspects, the one or more SNPs are at positions 1665, 1746, 2953, 3262, 3631, 3817, 4007, 4020, 4194, 4664, 4695, 4917, or 4952 when compared to the nucleic acid sequence that is available under the GenBank accession number FJ445132.1. In some aspects, the one or more SNPs are A1665U, A1746G, G2953C, U3262C, G3631A, A3817G, A4007G, U4020C, A4194G, A4664G, U4695C, A4917G, or U4952C when compared to the nucleic acid sequence that is available under the GenBank accession number FJ445132.1. In some aspects, the human rhinovirus comprises SNPs at positions 1665, 1746, 2953, 3262, 3631, 3817, 4007, 4020, 4194, 4664, 4695, 4917, and 4952 when compared to the nucleic acid sequence that is available under the GenBank accession number FJ445132.1. In some aspects, the human rhinovirus comprises the SNPs A1665U, A1746G, G2953C, U3262C, G3631A, A3817G, A4007G, U4020C, A4194G, A4664G, U4695C, A4917G, and U4952C when compared to the nucleic acid sequence that is available under the GenBank accession number FJ445132.1.

In some aspects, the rhinovirus can be encoded by the nucleic acid sequence of SEQ ID NO:1, SEQ ID NO:3, or a variant thereof. In some aspects, a rhinovirus can comprise the amino acid sequence of SEQ ID NO:2, SEQ ID NO:4, or a variant thereof. In some aspects, a rhinovirus can be encoded by a nucleic acid sequence 70, 75, 80, 85, 90, 95, or 99% identical to SEQ ID NO: 1 or SEQ ID NO:3. In some aspects, a rhinovirus can comprise an amino acid sequence 70, 75, 80, 85, 90, 95, or 99% identical to of SEQ ID NO:2 or SEQ ID NO:4.

In some aspects, a combination of rhinoviruses can be used to treat a subject having cancer. In some aspects, two or more rhinoviruses can be present in a single composition. In some aspects, two separate compositions, each comprising a different rhinovirus, can be administered for the treatment of cancer. For example, a combination of HRVA2 and HRVA45 can be used in the disclosed methods. In some aspects, each of the HRVA2 and HRVA45 are variants of wildtype viruses identified by GenBank references X02316.1 or FJ445132.1. In some aspects, the combination of HRVA2 and HRVA45 can be any of those disclosed herein.

### 5. Methods of Increasing Survival

Disclosed are methods of increasing survival of a subject having cancer comprising administering to the subject one or more of the disclosed human rhinoviruses or one or more of the disclosed compositions comprising a human rhinovirus. In some aspects, the human rhinovirus is one or more of the variant human rhinoviruses disclosed herein.

In some aspects, the cancer can be a solid tumor. For example, in some aspects, the cancer can be head and neck cancer, skin cancer, breast cancer, or any HPV associated cancer. In some aspects, the cancer is multiple myeloma.

In some aspects, administering can be local or systemic administration.

In some aspects, the one or more human rhinoviruses, administered alone or as a composition, comprises one or more single nucleotide polymorphisms (SNPs), wherein the SNPs are in the nucleic acid sequences in the 5′UTR, VP4, VP3, VP1, P2-C polypeptide, P3-A polypeptide. VpG protein. Protease 3C, or RDRP regions of the rhinovirus when compared to the nucleic acid sequence that is available under the GenBank accession number X02316.1. In some aspects, the one or SNPs are at positions 228, 491, 740, 744, 1735, 2491, 2517, 2714, 4755, 4756, 4854, 5018, 5023, 5026, 5027, 5152, 5300, 5521, or 6238 when compared to the nucleic acid sequence that is available under the GenBank accession number X02316.1. In some aspects, the one or SNPs are A228U, U491C, C740A, A744G, A1735G, A2491U, G2517A, G2714A, GC4755/4756CG, A4854G, G5018A, G5023A, GA5026/5027AG, G5152A, A5300G, C5521U, or A6238G when compared to the nucleic acid sequence that is available under the GenBank accession number X02316.1. In some aspects, the human rhinovirus comprises SNPs at positions 228, 491, 740, 744, 1735, 2491, 2517, 2714, 4755, 4756, 4854, 5018, 5023, 5026, 5027, 5152, 5300, 5521, or 6238 when compared to the nucleic acid sequence that is available under the GenBank accession number X02316.1. In some aspects, the human rhinovirus comprises the SNPs A228U, U491C, C740A, A744G, A1735G, A2491U, G2517A, G2714A, GC4755/4756CG, A4854G, G5018A, G5023A, GA5026/5027AG, G5152A, A5300G, C5521U, and A6238G when compared to the nucleic acid sequence that is available under the GenBank accession number X02316.1.

In some aspects, the one or more human rhinoviruses, administered alone or as a composition, comprises one or more single nucleotide polymorphisms (SNP), wherein the SNPs are in the nucleic acid sequences in the VP3, VP1, P2-B polypeptide, P2-C polypeptide, or P3-A polypeptide regions when compared to the nucleic acid sequence that is available under the GenBank accession number FJ445132.1. In some aspects, the one or more SNPs are at positions 1665, 1746, 2953, 3262, 3631, 3817, 4007, 4020, 4194, 4664, 4695, 4917, or 4952 when compared to the nucleic acid sequence that is available under the GenBank accession number FJ445132.1. In some aspects, the one or more SNPs are A1665U, A1746G, G2953C, U3262C, G3631A, A3817G, A4007G, U4020C, A4194G, A4664G, U4695C, A4917G, or U4952C when compared to the nucleic acid sequence that is available under the GenBank accession number FJ445132.1. In some aspects, the human rhinovirus comprises SNPs at positions 1665, 1746, 2953, 3262, 3631, 3817, 4007, 4020, 4194, 4664, 4695, 4917, and 4952 when compared to the nucleic acid sequence that is available under the GenBank accession number FJ445132.1. In some aspects, the human rhinovirus comprises the SNPs A1665U, A1746G, G2953C, U3262C, G3631A, A3817G, A4007G, U4020C, A4194G, A4664G, U4695C, A4917G, and U4952C when compared to the nucleic acid sequence that is available under the GenBank accession number FJ445132.1.

In some aspects, the rhinovirus can be encoded by the nucleic acid sequence of SEQ ID NO:1, SEQ ID NO:3, or a variant thereof. In some aspects, a rhinovirus can comprise the amino acid sequence of SEQ ID NO:2, SEQ ID NO:4, or a variant thereof. In some aspects, a rhinovirus can be encoded by a nucleic acid sequence 70, 75, 80, 85, 90, 95, or 99% identical to SEQ ID NO: 1 or SEQ ID NO:3. In some aspects, a rhinovirus can comprise an amino acid sequence 70, 75, 80, 85, 90, 95, or 99% identical to of SEQ ID NO:2 or SEQ ID NO:4.

In some aspects, a combination of rhinoviruses can be used to treat a subject having cancer. In some aspects, two or more rhinoviruses can be present in a single composition. In some aspects, two separate compositions, each comprising a different rhinovirus, can be administered for the treatment of cancer. For example, a combination of HRVA2 and HRVA45 can be used in the disclosed methods. In some aspects, each of the HRVA2 and HRVA45 are variants of wildtype viruses identified by GenBank references X02316.1 or FJ445132.1. In some aspects, the combination of HRVA2 and HRVA45 can be any of those disclosed herein.

Also disclosed are methods of increasing survival of a subject having cancer comprising administering to the subject one or more of the disclosed human rhinoviruses or one or more of the disclosed compositions comprising a human rhinovirus and further comprising administering a known anti-cancer therapeutic to the subject having cancer. In some aspects, the known anti-cancer therapeutic is an immune checkpoint inhibitor. In some aspects, an immune checkpoint inhibitor can be a CTLA-4 inhibitor, PD-1 inhibitor or PD-L1 inhibitor.

In some aspects, survival is increased due to the human rhinovirus killing cancer cells and/or increasing the immune response (e.g. pro-inflammatory cytokines).

### 6. Methods of Treating Viral Induced Lesions

Disclosed are methods of treating viral induced lesions in a subject comprising administering to a subject in need thereof a composition comprising a human rhinovirus. In some aspects, the human rhinovirus is one or more of the variant human rhinoviruses disclosed herein. In some aspects, the viral induced lesions are on the skin of the subject.

In some aspects, a viral induced lesion can be a wart. Disclosed are methods of treating warts in a subject comprising administering to a subject in need thereof a composition comprising a human rhinovirus. In some aspects, the human rhinovirus is one or more of the variant human rhinoviruses disclosed herein.

Disclosed are methods for treating viral induced lesions of the skin, including HPV driven papillomas in a subject, the method comprising administering a therapeutically effective amount of HRVA2.s and/or HRVA45.s alone or in combination with immunomodulatory agents such that some cells of the papilloma cells undergo selective viral lysis, wherein the host immune system is engaged culminating in host immune activation and papilloma in the subject.

Disclosed are methods of treating a hematologic or a solid cancers in a subject, the method comprising administering locally or systemically a therapeutically effective amount of one or more of the compositions disclosed herein.

Disclosed are methods of treating viral induced lesions of the skin, including HPV driven papillomas in a subject, the method comprising administering a therapeutically effective amount of one or more of the compositions disclosed herein.

Disclosed are methods of treating viral induced lesions of the skin, including HPV driven papillomas in a subject, the method comprising administering a therapeutically effective amount of one or more of the compositions disclosed herein alone or in combination with immunomodulatory agents such that some cells of the papilloma cells undergo selective viral lysis, wherein the host immune system is engaged culminating in host immune activation and papilloma in the subject.

In some aspects, administering can be local or systemic administration.

In some aspects, the one or more human rhinoviruses, administered alone or as a composition, comprises one or more single nucleotide polymorphisms (SNPs), wherein the SNPs are in the nucleic acid sequences in the 5′UTR, VP4, VP3, VP1, P2-C polypeptide, P3-A polypeptide. VpG protein. Protease 3C, or RDRP regions of the rhinovirus when compared to the nucleic acid sequence that is available under the GenBank accession number X02316.1. In some aspects, the one or SNPs are at positions 228, 491, 740, 744, 1735, 2491, 2517, 2714, 4755, 4756, 4854, 5018, 5023, 5026, 5027, 5152, 5300, 5521, or 6238 when compared to the nucleic acid sequence that is available under the GenBank accession number X02316.1. In some aspects, the one or SNPs are A228U, U491C, C740A, A744G, A1735G, A2491U, G2517A, G2714A, GC4755/4756CG, A4854G, G5018A, G5023A, GA5026/5027AG, G5152A, A5300G, C5521U, or A6238G when compared to the nucleic acid sequence that is available under the GenBank accession number X02316.1. In some aspects, the human rhinovirus comprises SNPs at positions 228, 491, 740, 744, 1735, 2491, 2517, 2714, 4755, 4756, 4854, 5018, 5023, 5026, 5027, 5152, 5300, 5521, or 6238 when compared to the nucleic acid sequence that is available under the GenBank accession number X02316.1. In some aspects, the human rhinovirus comprises the SNPs A228U, U491C, C740A, A744G, A1735G, A2491U, G2517A, G2714A, GC4755/4756CG, A4854G, G5018A, G5023A, GA5026/5027AG, G5152A, A5300G, C5521U, and A6238G when compared to the nucleic acid sequence that is available under the GenBank accession number X02316.1.

In some aspects, the one or more human rhinoviruses, administered alone or as a composition, comprises one or more single nucleotide polymorphisms (SNP), wherein the SNPs are in the nucleic acid sequences in the VP3, VP1, P2-B polypeptide, P2-C polypeptide, or P3-A polypeptide regions when compared to the nucleic acid sequence that is available under the GenBank accession number FJ445132.1. In some aspects, the one or more SNPs are at positions 1665, 1746, 2953, 3262, 3631, 3817, 4007, 4020, 4194, 4664, 4695, 4917, or 4952 when compared to the nucleic acid sequence that is available under the GenBank accession number FJ445132.1. In some aspects, the one or more SNPs are A1665U, A1746G, G2953C, U3262C, G3631A, A3817G, A4007G, U4020C, A4194G, A4664G, U4695C, A4917G, or U4952C when compared to the nucleic acid sequence that is available under the GenBank accession number FJ445132.1. In some aspects, the human rhinovirus comprises SNPs at positions 1665, 1746, 2953, 3262, 3631, 3817, 4007, 4020, 4194, 4664, 4695, 4917, and 4952 when compared to the nucleic acid sequence that is available under the GenBank accession number FJ445132.1. In some aspects, the human rhinovirus comprises the SNPs A1665U, A1746G, G2953C, U3262C, G3631A, A3817G, A4007G, U4020C, A4194G, A4664G, U4695C, A4917G, and U4952C when compared to the nucleic acid sequence that is available under the GenBank accession number FJ445132.1.

In some aspects, the rhinovirus can be encoded by the nucleic acid sequence of SEQ ID NO:1, SEQ ID NO:3, or a variant thereof. In some aspects, a rhinovirus can comprise the amino acid sequence of SEQ ID NO:2, SEQ ID NO:4, or a variant thereof. In some aspects, a rhinovirus can be encoded by a nucleic acid sequence 70, 75, 80, 85, 90, 95, or 99% identical to SEQ ID NO: 1 or SEQ ID NO:3. In some aspects, a rhinovirus can comprise an amino acid sequence 70, 75, 80, 85, 90, 95, or 99% identical to of SEQ ID NO:2 or SEQ ID NO:4.

In some aspects, a combination of rhinoviruses can be used to treat viral induced lesions. In some aspects, two or more rhinoviruses can be present in a single composition. In some aspects, two separate compositions, each comprising a different rhinovirus, can be administered for the treatment of viral induced lesions. For example, a combination of HRVA2 and HRVA45 can be used in the disclosed methods. In some aspects, each of the HRVA2 and HRVA45 are variants of wildtype viruses identified by GenBank references X02316.1 or FJ445132.1. In some aspects, the combination of HRVA2 and HRVA45 can be any of those disclosed herein.

In some aspects, the human rhinovirus induces pro-inflammatory cytokines. In some aspects, pro-inflammatory cytokines can regulate growth, cell activation, differentiation, and homing of immune cells to the sites of infection with the aim to control and eradicate the intracellular pathogens, including viruses

In some aspects, viral induced lesions can be from, but are not limited to, herpes simplex virus, herpes zoster, human papilloma virus, vesicular stomatitis virus, or epstein-barr virus.

### 7. Methods of Killing Cells Infected with Virus

Disclosed are methods of killing a cell infected with a virus comprising contacting a cell infected with a virus with a human rhinovirus or a composition comprising a human rhinovirus. In some aspects, contacting a cell infected with a virus can include exposing a cell infected with a virus to a composition comprising a human rhinovirus or administering a composition comprising a human rhinovirus to a cell infected with a virus or a container comprising a cell infected with a virus.

In some aspects, the human rhinovirus induces an immune response, thereby killing cells infected with a virus. In some aspects, the human rhinovirus increases proinflammatory response, thereby killing cells infected with a virus.

In some aspects, the virus can be a virus that induces lesions, such as but not limited to, herpes simplex virus, herpes zoster, human papilloma virus, vesicular stomatitis virus, or epstein-barr virus.

In some aspects, the cell infected with a virus is in a subject. If the cell infected with a virus is in a subject, the method of killing the cell infected with a virus comprises administering a human rhinovirus or a composition comprising a human rhinovirus to a subject infected with the virus. In some aspects, the cell infected with a virus is in vitro. The in vitro methods comprise contact a cell infected with a virus with a human rhinovirus or a composition comprising a human rhinovirus or exposing a cell infected with a virus to a human rhinovirus or a composition comprising a human rhinovirus. In some aspects, the in vitro method comprises administering a human rhinovirus or a composition comprising a human rhinovirus to a container (e.g. culture well or plate) comprising cells infected with a virus.

In some aspects, the one or more human rhinoviruses, contacted to a cell infected with a virus alone or as a composition, comprises one or more single nucleotide polymorphisms (SNPs), wherein the SNPs are in the nucleic acid sequences in the 5′UTR, VP4, VP3, VP1, P2-C polypeptide, P3-A polypeptide. VpG protein. Protease 3C, or RDRP regions of the rhinovirus when compared to the nucleic acid sequence that is available under the GenBank accession number X02316.1. In some aspects, the one or SNPs are at positions 228, 491, 740, 744, 1735, 2491, 2517, 2714, 4755, 4756, 4854, 5018, 5023, 5026, 5027, 5152, 5300, 5521, or 6238 when compared to the nucleic acid sequence that is available under the GenBank accession number X02316.1. In some aspects, the one or SNPs are A228U, U491C, C740A, A744G, A1735G, A2491U, G2517A, G2714A, GC4755/4756CG, A4854G, G5018A, G5023A, GA5026/5027AG, G5152A, A5300G, C5521U, or A6238G when compared to the nucleic acid sequence that is available under the GenBank accession number X02316.1. In some aspects, the human rhinovirus comprises SNPs at positions 228, 491, 740, 744, 1735, 2491, 2517, 2714, 4755, 4756, 4854, 5018, 5023, 5026, 5027, 5152, 5300, 5521, or 6238 when compared to the nucleic acid sequence that is available under the GenBank accession number X02316.1. In some aspects, the human rhinovirus comprises the SNPs A228U, U491C, C740A, A744G, A1735G, A2491U, G2517A, G2714A, GC4755/4756CG, A4854G, G5018A, G5023A, GA5026/5027AG, G5152A, A5300G, C5521U, and A6238G when compared to the nucleic acid sequence that is available under the GenBank accession number X02316.1.

In some aspects, the one or more human rhinoviruses, contacted to a cell infected with a virus alone or as a composition, comprises one or more single nucleotide polymorphisms (SNP), wherein the SNPs are in the nucleic acid sequences in the VP3, VP1, P2-B polypeptide, P2-C polypeptide, or P3-A polypeptide regions when compared to the nucleic acid sequence that is available under the GenBank accession number FJ445132.1. In some aspects, the one or more SNPs are at positions 1665, 1746, 2953, 3262, 3631, 3817, 4007, 4020, 4194, 4664, 4695, 4917, or 4952 when compared to the nucleic acid sequence that is available under the GenBank accession number FJ445132.1. In some aspects, the one or more SNPs are A1665U, A1746G, G2953C, U3262C, G3631A, A3817G, A4007G, U4020C, A4194G, A4664G, U4695C, A4917G, or U4952C when compared to the nucleic acid sequence that is available under the GenBank accession number FJ445132.1. In some aspects, the human rhinovirus comprises SNPs at positions 1665, 1746, 2953, 3262, 3631, 3817, 4007, 4020, 4194, 4664, 4695, 4917, and 4952 when compared to the nucleic acid sequence that is available under the GenBank accession number FJ445132.1. In some aspects, the human rhinovirus comprises the SNPs A1665U, A1746G, G2953C, U3262C, G3631A, A3817G, A4007G, U4020C, A4194G, A4664G, U4695C, A4917G, and U4952C when compared to the nucleic acid sequence that is available under the GenBank accession number FJ445132.1.

In some aspects, the rhinovirus can be encoded by the nucleic acid sequence of SEQ ID NO:1, SEQ ID NO:3, or a variant thereof. In some aspects, a rhinovirus can comprise the amino acid sequence of SEQ ID NO:2, SEQ ID NO:4, or a variant thereof. In some aspects, a rhinovirus can be encoded by a nucleic acid sequence 70, 75, 80, 85, 90, 95, or 99% identical to SEQ ID NO: 1 or SEQ ID NO:3. In some aspects, a rhinovirus can comprise an amino acid sequence 70, 75, 80, 85, 90, 95, or 99% identical to of SEQ ID NO:2 or SEQ ID NO:4.

In some aspects, a combination of rhinoviruses can be used to kill cells infected with a virus. In some aspects, two or more rhinoviruses can be present in a single composition. In some aspects, two separate compositions, each comprising a different rhinovirus, can be contacted to a cancer cell for killing a cancer cell. For example, a combination of HRVA2 and HRVA45 can be used in the disclosed methods. In some aspects, each of the HRVA2 and HRVA45 are variants of wildtype viruses identified by GenBank references X02316.1 or FJ445132.1. In some aspects, the combination of HRVA2 and HRVA45 can be any of those disclosed herein.

In some aspects, the cell infected with a virus expresses ICAM-1 and/or LDLR on the surface. HRVA2, and variants thereof, bind to LDLR on the surface of cells infected with a virus. HRVA45, and variants thereof, bind to ICAM1 on the surface of cells infected with a virus. In some aspects, ICAM-1 and/or LDLR can first be upregulated in a cell infected with a virus in a subject prior to administering the human rhinovirus or composition comprising a human rhinovirus.

### E. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of the disclosed rhinoviruses.

### EXAMPLES

### A. Example 1—Treating Cancer

Synthetic genomes (GeneUniversal) of HRV A2 (based on X02316.1) and HRV A45 (based on FMDV2A) were generated following standard full length in vitro RNA transcription (Invitrogen), transfected H1-HELA cells (cervical cancer cell line that also harbors 3+ known HPVs) at 37 C in order to produce infectious virus. Viral titers of each, following initial harvest was <1e3 infectious units (IFU) per mL. Following standard purification (lyse infected cells/3 freeze-thaw cycles/remove cellular debris/sucrose gradient ultracentrifugation) each virus was used to infect fresh H1-HELA cells. This process was carried out weekly until viral titers reached ≥1e9 ifu/mL using a modified TCID₅₀ assay employing RT-PCR methodology. For HRVA45 this process took 12 months while it took 14 months for HRVA2. The titers of these selected viruses have since improved the titers to ˜1e11 ifu/mL by identifying optional MOI (0.01), harvest timing (22 h) and volume (125 mL) of each shaker flask at 37 C. Next Generation Sequencing (NGS) with complete coverage revealed numerous alterations within the 5′ untranslated (UTR) regulatory and coding sequence (CDS) of each HRV. These selected alterations are distinct from the initial synthetic genomes and other reported genome sequences of HRVA2 and HRVA45. Each has retained restricted tropism to cells expressing the appropriate cellular receptor ICAM1 (HRV A45) or LDLR (HRV A2).

HRVA2.s was found to have 19 accrued, unique base pair alterations with 11 different amino acids identified relative to the synthetic wild-type reference GenBank: X02316.1 at the following loci (See Table 1). All other reported HRVA2 variants have significantly greater divergence.

TABLE 1
HRVA2.s accrued alterations following selection

| Genomic | | | | NGS | | |
|---|---|---|---|---|---|---|
| position | HRVA2 | | | depth | AA | |
| (1-7102) | X02316.1 | HRVA2.s | Type | (reads) | change | Region |
| 228 | A | U | SNP | 5313 | N/A | 5′ UTR |
| 491 | U | C | SNP | 12307 | N/A | 5′ UTR |
| 740 | C | A | SNP | 16092 | L44M | VP4 |
| 744 | A | G | SNP | 15726 | E45G | VP4 |
| 1735 | A | G | SNP | 23517 | — | VP3 |
| 2491 | A | U | SNP | 33544 | Q627H | VP1 |
| 2517 | G | A | SNP | 34276 | S636N | VP1 |
| 2714 | G | A | SNP | 37908 | D701N | VP1 |
| 4755/4756 | GC | CG | MNP | 61869 | S1382T | P2-C |
| | | | | | | polypeptide |
| 4854 | A | G | SNP | 73294 | K1415R | P3-A |
| | | | | | | polypeptide |
| 5018 | G | A | SNP | 70450 | G1470S | P3-A |
| | | | | | | polypeptide |
| 5023 | G | A | SNP | 70458 | A1472T | P3-A |
| | | | | | | polypeptide |
| 5026/5027 | GA | AG | MNP | 70459 | R1473G | P3-A |
| | | | | | | polypeptide |
| 5152 | G | A | SNP | 70458 | — | VpG protein |
| 5300 | A | G | SNP | 46841 | I1563V | protease 3C |
| 5521 | C | U | SNP | 78513 | — | RDRP |
| 6238 | A | G | SNP | 89813 | — | RDRP |

The HRVA45.s strain has 13 accrued, unique base pair alterations with 7 different amino acids were identified relative to the synthetic wild-type reference GenBank: FJ445132.1 at the following loci (See Table 2). All other reported HRVA45 variants have significantly greater divergence.

TABLE 2
HRVA45.s accrued alterations following selection

| Genomic | | | | NGS | | |
|---|---|---|---|---|---|---|
| position | HRVA45 | | | depth | AA | |
| (1-7114) | FJ445132.1 | HRVA45.s | Type | (reads) | change | Region |
| 1665 | A | U | SNP | 11403 | — | VP3 |
| 1746 | A | G | SNP | 18423 | — | VP3 |
| 2953 | G | C | SNP | 33160 | D780H | VP1 |
| 3262 | U | C | SNP | 33227 | Y883H | Protease |
| | | | | | | P2A |
| 3631 | G | A | SNP | 36242 | D1006N | P2-B |
| | | | | | | poly- |
| | | | | | | peptide |
| 3817 | A | G | SNP | 36079 | N1068D | P2-B |
| | | | | | | poly- |
| | | | | | | peptide |
| 4007 | A | G | SNP | 41111 | N1131S | P2-C |
| | | | | | | poly- |
| | | | | | | peptide |
| 4020 | U | C | SNP | 32897 | — | P2-C |
| | | | | | | poly- |
| | | | | | | peptide |
| 4194 | A | G | SNP | 32345 | — | P2-C |
| | | | | | | poly- |
| | | | | | | peptide |
| 4664 | A | G | SNP | 21819 | K1350R | P2-C |
| | | | | | | poly- |
| | | | | | | peptide |
| 4695 | U | C | SNP | 21966 | — | P2-C |
| | | | | | | poly- |
| | | | | | | peptide |
| 4917 | A | G | SNP | 32849 | — | P3-A |
| | | | | | | poly- |
| | | | | | | peptide |
| 4952 | U | C | SNP | 25976 | I1446T | P3-A |
| | | | | | | poly- |
| | | | | | | peptide |

Utilizing the National Library of Medicine BLASTn tool reveals 11 HRVA2 reported genomes with X02316.1 having the greatest identity to HRVA2.s at 99% (7083/7102). HRVA45.s BLASTn results in HRV A45 FJ445132.1 having the greatest identity to HRVA45.s at 99% (7101/7114). Below are the unique HRVA2.s and HRVA45.s unique RNA nucleotide sequences with alterations bolded along with the amino acid sequences, also with selected alterations bolded.

HRVA2.s nucleotide sequence
(SEQ ID NO: 1)
UUAAAACUGGAUCCAGGUUGUUCCCACCUGGAUUUCCCACAGGGA
GUGGUACUCUGUUAUUACGGUAACUUUGUACGCCAGUUUUAUCUC
CCUUCCCCCAUGUAACUUAGAAGUUUUUCACAAAGACCAAUAGCC
GGUAAUCAGCCAGAUUACUGAAGGUCAAGCACUUCUGUUUCCCCG
GUCAAUGUUGAUAUGCUCCAACAGGGCAAAAACAACUGCGAUCGU
UAUCCGCAAAGCGCCUACGCAAAGCUUAGUAGCAUCUUUGAAAUC
GUUUGGCUGGUCGAUCCGCCAUUUCCCCUGGUAGACCUGGCAGAU
GAGGCUAGAAAUACCCCACUGGCGACAGUGUUCUAGCCUGCGUGG
CUGCCUGCACACCCUAUGGGUGUGAAGCCAAACAAUGGACAAGGU
GUGAAGAGCCCCGUGUGCUCGCUUUGAGUCCUCCGGCCCCUGAAU
GUGGCUAACCUUAACCCUGCAGCUAGAGCACGUAACCCAACGUGU
AUCUAGUCGUAAUGAGCAAUUGCGGGAUGGGACCAACUACUUUGG
GUGUCCGUGUUUCACUUUUUCCUUUAUAUUUGCUUAUGGUGACAA
UAUAUACAAUAUAUAUAUUGGCACCAUGGGUGCACAGGUUUCAAG
ACAAAAUGUUGGAACUCACUCCACGCAAAACUCUGUAUCAAAUGG
GUCUAGUUUAAAUUAUUUUAACAUCAAUUAUUUCAAAGAUGCUGC
UUCAAAUGGUGCAUCAAAAAUGGGAUUCACACAAGAUCCUAGUAA
AUUUACUGACCCAGUUAAGGAUGUUUUGGAAAAGGGAAUACCAAC
ACUACAGUCCCCCACAGUGGAGGCUUGUGGAUACUCUGAUAGGAU
UAUACAGAUUACCAGAGGAGAUUCAACCAUAACCUCACAAGAUGU
GGCUAAUGCUAUCGUUGCGUAUGGUGUUUGGCCACAUUAUCUAUC
CUCCAAGGAUGCCUCUGCAAUUGAUAAACCCUCUCAACCAGAUAC
AUCUUCUAAUAGAUUUUAUACUCUAAGGAGUGUGACCUGGAGCAG
UUCCUCAAAGGGUUGGUGGUGGAAACUACCUGAUGCACUCAAGGA
CAUGGGUAUUUUUGGUGAAAACAUGUUUUAUCAUUACCUGGGUAG
GAGUGGAUACACAAUACAUGUGCAGUGUAAUGCUAGUAAAUUUCA
CCAGGGUACACUAAUUGUUGCUCUGAUACCUGAGCAUCAGAUUGC
AAGUGCCUUACAUGGCAAUGUGAAUGUUGGUUACAACUACACACA
CCCAGGUGAAACAGGCAGGGAAGUUAAAGCUGAGACGAGAUUGAA
UCCUGAUCUACAACCUACUGAAGAGUAUUGGCUAAACUUUGAUGG
GACACUCCUUGGAAAUAUUACCAUAUUCCCUCAUCAAUUUAUCAA
CUUGAGGAGUAAUAAUUCUGCCACAAUAAUUGCCCCUUAUGUCAA
UGCAGUUCCUAUGGAUUCAAUGCGGAGCCACAAUAAUUGGAGUUU
GGUAAUAAUACCAAUAUGUCCCCUUGAGACAUCAAGUGCAAUUAA
CACAAUACCUAUUACAAUAUCUAUAAGCCCCAUGUGUGCAGAGUU
UUCCGGCGCGCGUGCCAAGCGUCAAGGAUUACCAGUUUUCAUCAC
ACCAGGUUCAGGACAGUUUUUGACAACAGAUGAUUUCCAAUCCCC
AUGUGCACUUCCCUGGUAUCACCCAACUAAGGAAAUUUCUAUUCC
AGGUGAGGUUAAAAAUUUGGUUGAGAUUUGUCAAGUAGACAGCCU
AGUACCAAUAAAUAACACUGACACCUACAUCAAUAGUGAAAAUAU
GUAUUCUGUUGUAUUGCAAUCAUCAAUUAAUGCACCAGAUAAGAU
CUUCUCUAUUCGAACAGAUGUUGCUUCCCAACCUUUAGCUACUAC
UUUGAUUGGUGAGAUAUCUAGCUAUUUCACCCACUGGACAGGGAG
UCUCCGUUUCAGCUUCAUGUUUUGUGGUACUGCCAACACUACUGU
UAAGCUUUUGUUGGCAUACACACCACCUGGUAUCGCAGAACCCAC
CACAAGAAAGGAUGCAAUGCUAGGCACUCAUGUUAUAUGGGAUGU
GGGGUUGCAGUCUACAAUAUCAAUGGUAGUGCCAUGGAUUAGCGC
UAGUCAUUAUAGAAACACAUCACCAGGUAGAUCUACAUCUGGGUA
CAUAACAUGCUGGUAUCAGACUAGAUUAGUCAUUCCACCUCAGAC
CCCACCAACAGCUAGAUUGUUAUGUUUUGUAUCUGGGUGCAAAGA
CUUUUGCUUGCGCAUGGCACGAGAUACUAACCUACACCUGCAAAG
UGGUGCAAUAGCACAGAACCCUGUUGAGAAUUAUAUAGAUGAAGU
UCUUAAUGAAGUUUUAGUUGUCCCAAAUAUUAAUAGUAGUAACCC
CACAACAUCAAAUUCUGCCCCAGCAUUAGAUGCUGCAGAAACAGG
GCACACUAGUAGUGUUCAACCAGAGGAUGUCAUUGAAACUAGGUA
UGUGCAGACAUCACAUACAAGAGAUGAAAUGAGUUUAGAGAAUUU
UCUUGGCAGAUCAGGAUGCAUACAUGAAUCUAAAUUAGAGGUUAC
ACUUGCAAAUUAUAACAAGGAGAAUUUUACAGUGUGGGCUAUUAA
UCUACAAGAAAUGGCUCAAAUUAGAAGGAAAUUUGAAUUGUUCAC
CUAUACUAGGUUUGAUUCUGAAAUAACCCUAGUUCCAUGCAUUUC
CGCCCUUAGUCAGAACAUUGGACACAUCACAAUGCAAUACAUGUA
UGUUCCACCAGGUGCACCGGUGCCCAAUAGUAGGGACGAUUAUGC
AUGGCAGUCUGGCACUAAUGCCUCUGUUUUCUGGCAACAUGGACA
GGCUUAUCCAAGAUUUUCCUUACCUUUCCUAAGUGUGGCAUCUGC
UUAUUACAUGUUUUAUGAUGGGUAUGAUGAACAAGAUCAAAACUA
UGGUACAGCAAACACAAAUAACAUGGGGUCACUAUGCUCUAGGAU
AGUAACAGAGAAACACAUUCAUAAAGUACAUAUAAUGACAAGAAU
CUAUCACAAGGCUAAACAUGUCAAGGCAUGGUGUCCACGCCCACC
CAGAGCGCUUGAGUAUACUCGUGCUCAUCGCACUAAUUUUAAAAU
UGAGGAUAGGAGUAUUCAGACAGCAAUUGUGACCAGACCAAUUAU
CACUACAGCUGGCCCCAGUGACAUGUAUGUUCAUGUAGGUAACCU
UAUUUAUAGAAAUCUUCAUCUUUUCAACUCUGAGAUGCAUGAAUC
UAUUUUGGUAUCUUAUUCAUCAGAUUUAAUCAUUUACCGAACAAA
CACUGUAGGUGAUGAUUACAUUCCCUCUUGUGAUUGUACCCAAGC
UACUUAUUAUUGCAAACAUAAAAAUAGAUACUUCCCAAUUACAGU
UACAAGCCAUGACUGGUAUGAAAUACAGGAAAGUGAGUACUAUCC
CAAACACAUACAGUACAAUUUGUUGAUUGGUGAGGGCCCUUGUGA
ACCAGGUGACUGUGGUGGAAAGUUGCUAUGCAAACAUGGUGUCAU
AGGUAUAGUAACAGCUGGUGGUGAUAAUCAUGUGGCUUUUAUUGA
CCUUAGACACUUCCAUUGUGCUGAAGAACAAGGGGUUACAGAUUA
UAUACAUAUGCUAGGAGAAGCAUUUGGAAAUGGAUUUGUGGAUAG
UGUAAAAGAACAUAUACAUGCCAUAAACCCAGUAGGAAAUAUCAG
CAAGAAAAUUAUUAAAUGGAUGUUGAGAAUAAUAUCAGCAAUGGU
CAUAAUAAUUAGAAACUCUUCUGACCCCCAAACUAUAUUAGCAAC
ACUCACACUGAUUGGGUGUUCUGGAUCACCCUGGAGAUUUUUAAA
GGAAAAAUUCUGUAAAUGGACACAGCUUAAUUAUAUACACAAAGA
AUCAGAUUCAUGGUUAAAGAAAUUUACUGAAGCAUGCAAUGCAGC
UAGAGGGCUUGAAUGGAUAGGGAAUAAGAUAUCUAAAUUUAUUGA
AUGGAUGAAGUCGAUGCUCCCGCAAGCUCAAUUGAAGGUUAAGUA
CUUAAACGAGCUUAAAAAACUCAACCUAUACGAAAAGCAAGUUGA
GAGCUUGCGGGUGGCUGACAUGAAAACACAAGAAAAAAUUAAAAU
GGAAAUAGACACUUUACAUGAUUUGUCACGUAAAUUUCUACCUUU
GUAUGCAAGUGAGGCAAAAAGGAUAAAAACCCUAUACAUUAAAUG
UGAUAAUAUCAUCAAGCAGAAGAAAAGAUGUGAACCAGUAGCUAU
AGUUAUUCAUGGACCACCUGGUGCUGGCAAAUCUAUAACAACAAA
UUUCCUGGCCAAAAUGAUAACUAAUGAUAGUGACAUAUACUCUCU
ACCUCCUGAUCCAAAAUAUUUUGAUGGUUAUGACCAACAGAGUGU
AGUAAUAAUGGAUGACAUUAUGCAGAAUCCAGCCGGGGAUGACAU
GACACUGUUCUGCCAAAUGGUUUCUAGUGUUACAUUUAUACCACC
AAUGGCUGAUCUACCAGAUAAAGGCAAGGCUUUUGAUUCUAGGUU
UGUAUUAUGCAGCACAAAUCAUUCCCUUCUAACACCCCCGACAAU
AACUUCACUACCUGCAAUGAAUAGAAGAUUUUUCCUAGAUUUAGA
UAUAAUAGUACAUGAUAACUUCAAAGAUCCACAGGGCAAACUUAA
UGUGGCAGCAGCGUUUCGACCAUGUGAUGUAGAUAAUAGAAUAGG
AAAUGCACGUUGUUGUCCAUUUGUGUGUGGAAAAGCAGUUUCUUU
CAAAGAUCGUAACUCUUGCAACAAAUACACGCUUGCGCAGGUGUA
CAACAUAAUGAUUGAAGAAGACAGACGGAGAAGACAAGUGGUUGA
UGUCAUGACAGCUAUAUUCCAAGGGCCAAUUGAUAUGAGAAACCC
ACCACCACCUGCUAUUACUGACUUGCUCCAGUCUGUUAGAACCCC
UGAAGUUAUUAAGUAUUGUGAGGGUAAUAGAUGGAUAAUUCCAGC
AGAAUGCAAGAUAGAAAAGGAGUUGAACUUGGCUAACACAAUCAU
AACAAUCAUUGCAAAUGUUAUUAGUAUGACAGGAAUAAUAUAUGU
UAUUUACAAACUUUUUUGCACAUUACAGGGACCAUAUUCAGGAGA
ACCAAAGCCCAAGACUAAAAUCCCAGAAAGGCGUGUAGUAACACA
GGGACCAGAGGAGGAAUUUGGAAUGUCUUUAAUUAAACAUAACUC
AUGUGUUAUUACAACAGAAAAUGGGAAAUUCACAGGUCUUGGAGU
AUACGACAGAUUUGUGGUCGUACCAACACAUGCAGAUCCUGGAAA
GGAAAUUCAGGUUGAUGGUAUAACUACAAAAGUCGUUGACUCAUA
UGACCUAUACAACAAGAAUGGGAUAAAGCUAGAAAUAACAGUACU
UAAAUUAGAUAGAAAUGAAAAAUUUAGAGAUAUCAGGAGAUAUAU
ACCUAACAAUGAAGAUGAUUACCCCAAUUGCAACUUAGCACUGCU
AGCAAACCAGCCUGAACCAACUAUAAUCAAUGUUGGAGAUGUUGU
AUCCUAUGGCAAUAUACUGCUCAGUGGCAAUCAAACGGCUAGAAU
GCUUAAAUACAGUUACCCAACUAAAUCUGGUUACUGUGGAGGUGU
CUUAUACAAAAUUGGGCAAGUGCUUGGAAUACAUGUUGGGGGCAA
UGGUAGGGAUGGUUUCUCAGCUAUGUUACUCAGAUCCUAUUUCAC
UGAUGUUCAGGGCCAAAUAACGUUAUCAAAGAAGACCAGUGAAUG
UAACCUACCCAGUAUACACACCCCAUGCAAAACCAAAUUGCAGCC
UAGUGUUUUCUAUGAUGUAUUCCCUGGUUCAAAAGAACCAGCUGU
GUUGUCUGAAAAAGAUGCCCGGUUACAAGUUGAUUUCAAUGAAGC
ACUAUUUUCUAAAUACAAAGGGAAUACAGAUUGCUCCAUUAAUGA
CCACAUAAGAAUUGCAUCAUCACAUUAUGCAGCACAACUCAUUAC
CUUAGAUAUUGACCCAAAACCUAUUACACUUGAGGACAGUGUCUU
UGGCACUGAUGGAUUAGAGGCUCUUGAUUUGAACACUAGCGCAGG
AUUUCCAUAUAUUGCAAUGGGAGUUAAAAAGAGAGAUUUAAUAAA
CAACAAGACCAAGGAUAUAAGCAAACUUAAAGAAGCAAUUGACAA
AUACGGAGUUGACUUACCUAUGGUCACCUUCUUGAAAGAUGAACU
CAGAAAGCAUGAAAAGGUAAUUAAAGGUAAAACUAGAGUUAUUGA
AGCUAGUAGUGUGAAUGAUACCCUAUUGUUUAGAACAACUUUUGG
CAACCUCUUUUCAAAGUUCCACUUGAAUCCUGGAAUUGUUACUGG
AUCAGCAGUUGGAUGUGAUCCAGAGGUGUUUUGGUCAAAAAUACC
AGCAAUGUUGGAUGAUAAAUGUAUUAUGGCUUUUGAUUAUACAAA
UUAUGAUGGUAGUAUACACCCUAUUUGGUUUGAAGCUCUUAAACA
GGUACUGGUAGAUCUAUCAUUUAAUCCAACAUUAAUAGAUAGACU
AUGCAAGUCUAAACACAUCUUCAAAAAUACAUACUAUGAAGUGGA
GGGAGGUGUACCAUCUGGGUGUUCAGGUACUAGUAUUUUUAACAC
UAUGAUCAAUAAUAUUAUCAUAAGGACCUUAGUGUUAGAUGCAUA
CAAGAAUAUAGAUCUAGAUAAGCUUAAGAUAAUUGCCUAUGGUGA
UGAUGUCAUAUUCUCAUACAUACAUGAACUGGACAUGGAGGCUAU
AGCAAUAGAGGGUGUUAAAUAUGGUUUGACUAUAACUCCUGCUGA
UAAAUCUAACACAUUUGUAAAAUUAGACUAUAGCAAUGUUACUUU
UUUAAAAAGAGGGUUUAAGCAAGAUGAGAAGUAUAACUUUCUAAU
ACAUCCAACUUUCCCUGAAGAUGAAAUAUUUGAAUCCAUCAGAUG
GACAAAGAAACCAUCACAAAUGCAUGAACAUGUGUUGUCUCUGUG
UCACUUAAUGUGGCACAAUGGACGUGACGCAUACAAAAAAUUUGU
GGAGAAGAUACGCAGUGUAAGCGCUGGUCGUGCACUGUACAUCCC
UCCGUAUGAUUUGCUUUUGCAUGAGUGGUAUGAAAAAUUUUAAAG
AUAUAGAAAUAGUAAACUGAUAGUUUAUUAGUUUUAU
HRVA2.s amino acid sequence
(SEQ ID NO: 2)
MGAQVSRQNVGTHSTQNSVSNGSSLNYFNINYFKDAASNGASKMG
FTQDPSKFTDPVKDVLEKGIPTLQSPTVEACGYSDRIIQITRGDS
TITSQDVANAIVAYGVWPHYLSSKDASAIDKPSQPDTSSNRFYTL
RSVTWSSSSKGWWWKLPDALKDMGIFGENMFYHYLGRSGYTIHVQ
CNASKFHQGTLIVALIPEHQIASALHGNVNVGYNYTHPGETGREV
KAETRLNPDLQPTEEYWLNFDGTLLGNITIFPHQFINLRSNNSAT
IIAPYVNAVPMDSMRSHNNWSLVIIPICPLETSSAINTIPITISI
SPMCAEFSGARAKRQGLPVFITPGSGQFLTTDDFQSPCALPWYHP
TKEISIPGEVKNLVEICQVDSLVPINNTDTYINSENMYSVVLQSS
INAPDKIFSIRTDVASQPLATTLIGEISSYFTHWTGSLRFSFMFC
GTANTTVKLLLAYTPPGIAEPTTRKDAMLGTHVIWDVGLQSTISM
VVPWISASHYRNTSPGRSTSGYITCWYQTRLVIPPQTPPTARLLC
FVSGCKDFCLRMARDTNLHLQSGAIAQNPVENYIDEVLNEVLVVP
NINSSNPTTSNSAPALDAAETGHTSSVQPEDVIETRYVQTSHTRD
EMSLENFLGRSGCIHESKLEVTLANYNKENFTVWAINLQEMAQIR
RKFELFTYTRFDSEITLVPCISALSQNIGHITMQYMYVPPGAPVP
NSRDDYAWQSGTNASVFWQHGQAYPRFSLPFLSVASAYYMFYDGY
DEQDQNYGTANTNNMGSLCSRIVTEKHIHKVHIMTRIYHKAKHVK
AWCPRPPRALEYTRAHRTNFKIEDRSIQTAIVTRPIITTAGPSDM
YVHVGNLIYRNLHLFNSEMHESILVSYSSDLIIYRTNTVGDDYIP
SCDCTQATYYCKHKNRYFPITVTSHDWYEIQESEYYPKHIQYNLL
IGEGPCEPGDCGGKLLCKHGVIGIVTAGGDNHVAFIDLRHFHCAE
EQGVTDYIHMLGEAFGNGFVDSVKEHIHAINPVGNISKKIIKWML
RIISAMVIIIRNSSDPQTILATLTLIGCSGSPWRFLKEKFCKWTQ
LNYIHKESDSWLKKFTEACNAARGLEWIGNKISKFIEWMKSMLPQ
AQLKVKYLNELKKLNLYEKQVESLRVADMKTQEKIKMEIDTLHDL
SRKFLPLYASEAKRIKTLYIKCDNIIKQKKRCEPVAIVIHGPPGA
GKSITTNFLAKMITNDSDIYSLPPDPKYFDGYDQQSVVIMDDIMQ
NPAGDDMTLFCQMVSSVTFIPPMADLPDKGKAFDSRFVLCSTNHS
LLTPPTITSLPAMNRRFFLDLDIIVHDNFKDPQGKLNVAAAFRPC
DVDNRIGNARCCPFVCGKAVSFKDRNSCNKYTLAQVYNIMIEEDR
RRRQVVDVMTAIFQGPIDMRNPPPPAITDLLQSVRTPEVIKYCEG
NRWIIPAECKIEKELNLANTIITIIANVISMTGIIYVIYKLFCTL
QGPYSGEPKPKTKIPERRVVTQGPEEEFGMSLIKHNSCVITTENG
KFTGLGVYDRFVVVPTHADPGKEIQVDGITTKVVDSYDLYNKNGI
KLEITVLKLDRNEKFRDIRRYIPNNEDDYPNCNLALLANQPEPTI
INVGDVVSYGNILLSGNQTARMLKYSYPTKSGYCGGVLYKIGQVL
GIHVGGNGRDGFSAMLLRSYFTDVQGQITLSKKTSECNLPSIHTP
CKTKLQPSVFYDVFPGSKEPAVLSEKDARLQVDFNEALFSKYKGN
TDCSINDHIRIASSHYAAQLITLDIDPKPITLEDSVFGTDGLEAL
DLNTSAGFPYIAMGVKKRDLINNKTKDISKLKEAIDKYGVDLPMV
TFLKDELRKHEKVIKGKTRVIEASSVNDTLLFRTTFGNLFSKFHL
NPGIVTGSAVGCDPEVFWSKIPAMLDDKCIMAFDYTNYDGSIHPI
WFEALKQVLVDLSFNPTLIDRLCKSKHIFKNTYYEVEGGVPSGCS
GTSIFNTMINNIIIRTLVLDAYKNIDLDKLKIIAYGDDVIFSYIH
ELDMEAIAIEGVKYGLTITPADKSNTFVKLDYSNVTFLKRGFKQD
EKYNFLIHPTFPEDEIFESIRWTKKPSQMHEHVLSLCHLMWHNGR
DAYKKFVEKIRSVSAGRALYIPPYDLLLHEWYEKF*
HRVA45.s nucleotide sequence
(SEQ ID NO: 3)
UUAAAACUGGGUCGUGGUUGUUCCCACCACGACUAUCUACGGAUG
UAGUGCUCUUGUAUUCCGGUACGCUUGCACGCCAGUUUUGUCACC
CCCCCCUACAUUGUAACUUAGAAGAUUAACACAAAGACCAAUAGG
CGGCAAUGAACCACAUUGUCAACGGUCAAGCACUUCUGUUUCCCC
GGUCAAUCUAGAUAUGCUUUACCAAAGGCAAAAACUGGAGCGAUC
GUUAUCCGCAAGGUGCCUACGGGAAACCCAGUAGCAUUCUUUAUU
UGAUCUGGUUGGUUGCUCAGCCGUUAAACCCAAACGGUAAACCUG
GCAGAUGAGGCUGGAAGAUCCCCACCAGCGAUGGUGUUCCAGCCU
GCGUGGCUGCCUGCACACCCGAAAGGGUGUGAAGCCUUUUCAAAG
ACAGGGUGCGAAGAGUCUACUGUGCUCACCUUGAUUCCUCCGGCC
CCUGAAUGUGGCUAAUCCUAACCCCGUAGCUGUGGUGUGCAAUCC
AGCACAUUCGCAGUCGUAAUGGGUAACUGCGGGAUGGAACCAACU
ACUUUGGGUGUCCGUGUUUCUCUUUUUCCUUUUAUAUAUGCUUAU
GGUGACAAUUAUACGGAUAUAGUUGUCAUCAUGGGCGCUCAAGUC
UCAAGGCAGAAUGUUGGUACCCACUCUACACAAAACACAGUUACA
GGAGGAUCCAGUCUUAAUUAUUUUAAUAUUAAUUACUUCAAAGAU
GCUGCUUCAUCUGGAGCGUCAAGGUUGGACUUUUCUCAAGACCCA
AGUAAAUUUACAGACCCAGUCAAAGAUGUUUUAACCAAGGGUAUC
CCAACCCUACAGUCACCUACAGUAGAAGCUUGUGGGUAUUCAGAU
AGAAUAAUACAAAUUACCAGAGGAGACACAACAAUAACAUCCCAG
GACAUUGCAAACGCUGUAGUUGGAUAUGGAGUUUGGCCUACUUAU
UUAGAUUCAAAGGAUGCCUCAGCUAUAGAUAAGCCCACACAACCA
GAUACUUCUGCAAAUAGGUUUUACACACUGGAAAGUAAGGAAUGG
ACUCCAGAUAGUAAAGGAUGGUGGUGGAAAUUACCAGAUGCACUC
AAAGAUAUGGGUGUUUUUGGUGAAAACAUGUUCUACCAUGCACUU
GGUAGGUCUGGUUACCUAAUUCAUGUUCAGUGCAAUGCAAGUAAA
UUUCAUUCAGGGACCCUUCUUGUUGUUGCUAUCCCAGAGCACCAG
UUGGCAUAUAUAGGCACAGGAAAUGUAACAGUAGGAUACAAACAC
ACCCAUCCUGGAGAGACUGGAAGAGUCAUAGCAACAACCACAGAC
AAACAGACAAGACAGCCAUCGUUUGACAGUUGGCUCAAUUGUAAU
GGUACCCUUUUAGGCAAUGCCUUAAUAUUUCCACAUCAAUUUAUC
AAUUUGAGAACAAACAAUGCUGCCACCUUGAUCCUGCCGUACGUA
AAUGCAACUCCCAUGGAUUCCAUGUUAAGACAUAAUAAUUGGUCA
UUACUAAUAGUGCCUGUAUCAGAACUAAGAGGUGACACCAGUAUA
CCAAUUACUGUAUCAAUCUCUCCCAUGGCAGCUGAAUUUUCUGGA
GCACGGAACCGCAGUGCACGUGUGGAAGGUUUACCAGUGAUGUUA
ACACCAGGAUCAGGACAAUUCUUGACAACUGAUGACAUGCAAUCU
CCAUCAGUUUUACCUUACUUCCACUCAACACAAGAAAUCUUCAUA
CCAGGAGAAGUUAAAAAUCUUAUUGAAUUGUGCCAGGUGGACACC
AUGGUACCAUUAAACAACUUGCACAUAAAUAAAAACAAGAUUGGA
AUGUAUGCUCUACCACUUACACGACAGAAUACACCAGCUGCUGAA
CUCUUUGCAAUGCCAGUCGACAUUACAUCUUCACCUUUAGCAACC
ACGCUUUUAGGAGAGAUUGCUUCUUAUUACACCAACUGGACAGGU
AGUAUAAGAUUGAGUUUCAUGUUUUGUGGCAGUGCAAACACCUCA
CUCAAGCUUCUUCUAGCAUAUACUCCACCUGGAGUGAGUAAACCA
ACCAGUAGGAGAGAAGCUAUGCUGGGCACCCAUCUUGUAUGGGAU
GUAGGUCUUCAAUCCACAUGUUCUUUAGUCAUCCCGUGGAUAUCA
GCAUCACAUUUUCGGAAUACCACGCCAGACACCUACUCAAAAGCU
GGUUAUGUGACAUGCUGGUAUCAGACAAAUUUCAUCACAGCACCA
AACACACCACCUACAGCUGACAUUAUUUGUCUGGUUUCAGCAUGU
AAGGAUUUUUGCUUACGCAUGGCUAGAGAUACUAACCAGCACACU
CAGCUUGGAGCAAUAGAGCAAAACCCUGUUGAACAAUUUGCAGAA
GCAGUCCUUGAUCAAGUAUUAGUAGUUCCAAACACUCGACCCAGC
GAUGGGUUGAUUGCAAACUCAGCCCCAGCUUUGGAUGCAGCUGAA
ACUGGACACACCAGUUCAGUGCAGCCUGAGGACCUUAUAGAGACU
AGAUAUGUGAUUGCAGACCAAACCAGACAUGAAACCUCCAUUGAA
UCUUUUUUGGGUAGGGCUGGAUGUGUGGCCACUAUUAGUUUAGAC
AUUAACCAUGAUGACUACCAAAAGAAUUACAAAAAUUGGGCAAUU
AGUUUACAAGAAAUGUCACAAAUUAGGAGGAAAUUUGAAAUGUUU
ACAUAUGUCAGAUUUGAUUCCGAAAUAACAAUAGUACCAUGUGUU
GCUGCCACAGAAGGUAACUUGGGACACAUUGUUGUGCAAUACAUG
UUUGUACCACCAGGAGCACCUCUCCCUGUUAGUAGAACUGACAAC
ACUUGGCAAUCUAGCACAAAUGCAUCAGUCUUUUGGCAGGUUGGU
CAAACUUAUCCCAGAUUUUCUAUACCUUUCUCAAGUAUAGCUUCA
GCUUACUACAUGUUUUAUGAUGGAUACGACACUGAUGGCACAGAU
GCAGUGUAUGGUGUUAGUGUGACUAACCAUAUGGGGACUAUAUGU
GUUAGAAUUGUUACAGACCAACAACAACAUAGAGUUAAGAUCGAC
UCCAUGGUAUAUCUAAAAGCUAAACACAUCAAGGCAUGGUGUCCC
AGACCUCCAAGAGCAGUCACAUAUAACCAUACAUAUAAUCCAAAU
UAUGUUAGGGCUGAUGAAACAGCCACAAAAGUCCAAACUAGAGCA
AAUGUCACAACAGUAGGUCCAUCAGACAUGUUUAUUCACGCCUCA
GAGUUUUUAUACAGAAAUUACCAUCUCACUCCAGAAAAGGAACUG
AAAGAAGCAUGCCAAAUUGUACAUACUGCAGAUCUAGUUAUACAC
CGCACAAGAGACAAAGGUGAUGACUAUAUCCCACAAUGCAAUUGU
ACAGAUUGCUGUUACUACUGUGCCCACAAAGACAGGUAUAUUCCC
AUCAAAGUAGAAUACCAUAGCUACUACACCAUCCAGAAAUCAGAU
UAUUAUCCAAAACAUAUACAGUAUGAUAUACUAAUUGGUGAGGGA
CCUUCGCAACCAGGUGAUUGUGGAGGAAAACUUUUAUGCAGACAU
GGUGUUAUUGGUAUGGUGACAGCUGGAGGGGAAGGACAUGUAGCA
UUUACUGAUUUGAGGAAGUAUAGAAUGGUAGAGGCUGAGGAGCAA
GGUAUAACAGAUUAUGUUAAAUCCUUAGGUAAUGCUUUUGGAGUA
GGAUUUGUAGAUCAAAUUAAAGAACAAAUUAAUAAUAUAAACCCA
UUAAAUAAAAUCAGUGCUAAAGUGAUCAAAUGGCUAAUCAGAGUA
AUAUCAGCACUAGUGAUAGCAGUGCGUAGCCAAGGGGAUCCAGCA
ACACUAUCAGCUACUCUACUUUUACUUGGGUGCUCUGAUUCCCCG
UGGCGGUUCCUGAAACAGAAGGUGUGUACAUGGUUGGGGCUUAGA
UACAUACACAAAGAAUCAGAUGGUUGGAUCAAGAAAUUUACUGAA
AUGUGCAAUGCAGCCAGAGGCUUAGAAUGGAUAGGGUGUAAGAUU
UCAAAAUUCAUUGACUGGCUGAAAUCCAUGCUACCUCAAGCACAG
AGCAAAAUCAAGUUCCUCCACUUCAUGAAACAAUUGCAGCUAAAA
GAAAAACAGAUCGAUGGUUUACCUUAUGCAACAGUUAAGCAACAA
GAAGAUUAUCUCAAAGAAAUGGAAGAGAUGUUGGACAUUUCAAAU
AAACUAUUACCACUAUACCCAAAGGAGAAUAAGAUUAUAAAAGAU
CUACUCAAGCAAGCUAAAAGCAUGACAACAACAUCAAAGAGAGUU
GAACCAGUUGCAAUCAUGUUUCAUGGAGAUCCAGGGUCAGGGAAA
UCAGUGUGCACAAACAUCCUUGCCCGCAUGAUAACUAAUCCAUCA
GACAUAUAUUCCCUACCCCCAAAUCCAAAGUAUUUUGAUGGAUAC
CAUCAACAGACUGUAGUAAUAAUGGAUGAUGUGAUGCAAAACCCA
GAUGGGGAAGACAUGAGCACCUUCUGCCAAAUGGUUUCCUCAGUU
AAUUAUGUAGUGCCAAUGGCUGAUCUACCAGAUAAGGGUACCCUG
UUUUCAUCAGAUUAUGUCUUUUGCAGUACAAAUCAGCAUGUUUUG
AUUCCUCCAACCAUAUCUACCAUACCCGCCCUGAAUCGGCGAUUG
UUCUUCGAUCUAACUGUUAAGGUGAACCCCAGAUAUCAGGAAGCA
GGAAAACUAAAUCUGGACUGUGCAUUAAGACCAUGCAAUCAUGAG
UUAAAGGUUGGAAACGCAAGGUGUUGUCCUCUCAUUUGUGGUAAG
GCUAUAACCUUCAUUAACAGACACAACAAUGAAGAAUUGACACUG
UCCCAAAUAUACAAUCAAGUAGUAAAUGAACACAACCGCAGAUUA
AACGUGUCAAAACACAUGGAAGCAAUUUUCCAGGGACCUAUUGAU
AUGCAAGCACCUCCCCCACCAGCAAUAGUUGAUUUACUUAGAUCC
ACCAGAAAUGAGGAUGUCAUCAACUACUGCAAAAACCAAAAUUGG
ACUAUUCCUGCUGACAUCAGUAUUGAGAGAGAAUUGAAUCUGGUA
AAUCUUUCAAUAUCAAUUCUGGCAAAUCUAAUUAGUGUGAUAGGA
AUCAUUUACAUUAUAUAUAAAUUAUUUGUAUCAUUACAAGGACCU
UAUAGUGGACUACCCACCAAGAAAAAAGUGAUUCCAGAGAAAAGA
GUGGUAGUACAAGGUCCUUCUACUGAAUUUGGCCUAAGUUUAAUA
AAACAUAACACAUGUAUUGUAGAAACAGAAAAUGGUAAGUUUACA
GGUUUAGGAAUAUACGAUAACCUAUUAGUCAUCCCCACACAUGCU
GAUCCCAGUAGAACUGUCAAGGUGGAUGGAGUGGAAACAGAAGUG
UUGGACUCUUACGAUUUGUACAACAAAGAAGGAGUAAAAUUAGAA
AUCACAGUUCUGAUCUUAUCCAGAAAUGAAAAGUUUAGGGAUAUU
AGAAAAUACAUACCAAAUUCUGAGGAUGAUUAUGUCAACUGUAAU
CUGGCUUUAGUUGCAAACCAAGAUAUGCCACAGAUACUGGAGGUC
GGAGAUGUUGUCUCUUAUGGCAAUAUACUCUUGAGUGGUAAUAAC
ACCGCCAGAAUGCUCAAAUAUGAAUACCCUACAAAAUCGGGUUUU
UGUGGAGGUGUUUUGUACAAGGUGGGUCAAGUAAUUGGCAUACAC
GUAGGUGGCAAUGGAAGACAAGGAUUCUCAGCCAUGUUACUUAAA
AGAUAUUUCAACCAACAACAAGGUCAGAUAAUUCUAAAGAAACCA
GUCAAGGAAGUUGAUUAUCCCAGUAUACAUACCCCUACUAAGACA
AAACUCCAACCAAGUGUUUUCCAUGAUAUUUUUCCCGGAGUUAAG
GAACCUGCAGUUCUAACAGAAAAAGACCCCCGGUUGGAAGUAGAC
UUGAACUCCUCUCUUUUUUCAAAAUAUGCUGGAAAUGUUAAUUUA
GAAAUGAAUGAGUACAUGAUUGUUGCAGCAUCCCAUUAUGCAUCA
CAACUUGAAACACUAGACAUAUCCAACCAGCAAAUGUCUAUUGAG
GAAUGUGUGUAUGGAACAGAUAACUUAGAAGCCUUGGAUUUAAAU
ACUAGUGCAGGAUUUCCCUAUGUGGCUUUGGGAAUUAAGAAGAAG
GACUUAAUAAAUAGAGAGACUAGAGAUGUCAGUAAAAUUAAAAAU
UGCUUAGACACUUAUGGUGUAGAUUUACCAAUGAUAACAUAUUUA
AAAGAUGAGCUCAGAACACCAGAAAAAAUAAAGUUAGGAAAAACU
AGGGUAAUAGAAGCAAGUAGUUUAAAUGAUACUAUUCAUAUGAGA
AUGCUCUUUGGAAACUUGUUUAAAGCCUUUCACGCAAAUCCAGGC
AUAGUAACAGGGUGUGCAGUUGGUUGUGAUCCAGAAACAUUUUGG
UCUAAAAUUCCUCCAAUGCUGGGUGAUGGGUGUGUGAUGGCAUUU
GAUUAUACAAAUUAUGAUGGAAGUUUACAUCCAAUAUGGUUUAGG
CUACUUGAAAGAGUGCUGGAUAGGUUGGGAUUCCCAGGCUGUGCA
GUUAGAAAAUUAUCACACUCCACCCAUAUAUACAAAGGAAUGUAC
UAUGAAGUUGAUGGAGGAAUGCCAUCUGGGUGUGCAGGUACUUCA
AUCUUCAACUCAAUGAUCAAUAACAUCAUAAUCAGAACAAUAAUU
CUACAUGCUUACAAGAAUAUAGACCUGGAUCAGUUGAGGAUACUU
ACAUAUGGUGAUGAUAUAAUUUUUACUUACCCAGAUAAACUAGAC
AUGGCUUAUUUAGCCCAAAUUGGAGAAAAAUAUGGUUUAAAAAUG
ACACCUGCAGACAAGUCAGAUACAUUUAAGGAUUUGGAUCUGAGU
ACAGCAACUUUCCUAAAAAGAGGCUUUAAACCUGACUCAAAACAC
CCAUUUUUAGUCCAUCCCAUAUAUCCAAUCCAGGAUAUCUAUGAA
UCAAUCAGAUGGACAAAGAAUCCCAGAUGUUUACAGGAGCAUGUG
CUCAGUUUGGCUCACUUAUGCUGGCAUAGUGGACCAGAGCAGUAU
GCUGAUUUUGUCAAGAGAAUCAGAUCAACAUCUGUUGGGAAGAAC
CUAUACAUACCAUCUUAUGAUGUACUACUUUAUGAGUGGUAUGAG
AAAUUUUAAGUUAAUAUAUACAGUUACUAUUUAGGUAGUUUGGGU
GUAU
HRVA45.s amino acid sequence
(SEQ ID NO: 4)
MGAQVSRQNVGTHSTQNTVTGGSSLNYFNINYFKDAASSGASRLD
FSQDPSKFTDPVKDVLTKGIPTLQSPTVEACGYSDRIIQITRGDT
TITSQDIANAVVGYGVWPTYLDSKDASAIDKPTQPDTSANRFYTL
ESKEWTPDSKGWWWKLPDALKDMGVFGENMFYHALGRSGYLIHVQ
CNASKFHSGTLLVVAIPEHQLAYIGTGNVTVGYKHTHPGETGRVI
ATTTDKQTRQPSFDSWLNCNGTLLGNALIFPHQFINLRTNNAATL
ILPYVNATPMDSMLRHNNWSLLIVPVSELRGDTSIPITVSISPMA
AEFSGARNRSARVEGLPVMLTPGSGQFLTTDDMQSPSVLPYFHST
QEIFIPGEVKNLIELCQVDTMVPLNNLHINKNKIGMYALPLTRQN
TPAAELFAMPVDITSSPLATTLLGEIASYYTNWTGSIRLSFMFCG
SANTSLKLLLAYTPPGVSKPTSRREAMLGTHLVWDVGLQSTCSLV
IPWISASHFRNTTPDTYSKAGYVTCWYQTNFITAPNTPPTADIIC
LVSACKDFCLRMARDTNQHTQLGAIEQNPVEQFAEAVLDQVLVVP
NTRPSDGLIANSAPALDAAETGHTSSVQPEDLIETRYVIADQTRH
ETSIESFLGRAGCVATISLDINHDDYQKNYKNWAISLQEMSQIRR
KFEMFTYVRFDSEITIVPCVAATEGNLGHIVVQYMFVPPGAPLPV
SRTDNTWQSSTNASVFWQVGQTYPRFSIPFSSIASAYYMFYDGYD
TDGTDAVYGVSVTNHMGTICVRIVTDQQQHRVKIDSMVYLKAKHI
KAWCPRPPRAVTYNHTYNPNYVRADETATKVQTRANVTTVGPSDM
FIHASEFLYRNYHLTPEKELKEACQIVHTADLVIHRTRDKGDDYI
PQCNCTDCCYYCAHKDRYIPIKVEYHSYYTIQKSDYYPKHIQYDI
LIGEGPSQPGDCGGKLLCRHGVIGMVTAGGEGHVAFTDLRKYRMV
EAEEQGITDYVKSLGNAFGVGFVDQIKEQINNINPLNKISAKVIK
WLIRVISALVIAVRSQGDPATLSATLLLLGCSDSPWRFLKQKVCT
WLGLRYIHKESDGWIKKFTEMCNAARGLEWIGCKISKFIDWLKSM
LPQAQSKIKFLHFMKQLQLKEKQIDGLPYATVKQQEDYLKEMEEM
LDISNKLLPLYPKENKIIKDLLKQAKSMTTTSKRVEPVAIMFHGD
PGSGKSVCTNILARMITNPSDIYSLPPNPKYFDGYHQQTVVIMDD
VMQNPDGEDMSTFCQMVSSVNYVVPMADLPDKGTLFSSDYVFCST
NQHVLIPPTISTIPALNRRLFFDLTVKVNPRYQEAGKLNLDCALR
PCNHELKVGNARCCPLICGKAITFINRHNNEELTLSQIYNQVVNE
HNRRLNVSKHMEAIFQGPIDMQAPPPPAIVDLLRSTRNEDVINYC
KNQNWTIPADISIERELNLVNLSISILANLISVIGIIYIIYKLFV
SLQGPYSGLPTKKKVIPEKRVVVQGPSTEFGLSLIKHNTCIVETE
NGKFTGLGIYDNLLVIPTHADPSRTVKVDGVETEVLDSYDLYNKE
GVKLEITVLILSRNEKFRDIRKYIPNSEDDYVNCNLALVANQDMP
QILEVGDVVSYGNILLSGNNTARMLKYEYPTKSGFCGGVLYKVGQ
VIGIHVGGNGRQGFSAMLLKRYFNQQQGQIILKKPVKEVDYPSIH
TPTKTKLQPSVFHDIFPGVKEPAVLTEKDPRLEVDLNSSLFSKYA
GNVNLEMNEYMIVAASHYASQLETLDISNQQMSIEECVYGTDNLE
ALDLNTSAGFPYVALGIKKKDLINRETRDVSKIKNCLDTYGVDLP
MITYLKDELRTPEKIKLGKTRVIEASSLNDTIHMRMLFGNLFKAF
HANPGIVTGCAVGCDPETFWSKIPPMLGDGCVMAFDYTNYDGSLH
PIWFRLLERVLDRLGFPGCAVRKLSHSTHIYKGMYYEVDGGMPSG
CAGTSIFNSMINNIIIRTIILHAYKNIDLDQLRILTYGDDIIFTY
PDKLDMAYLAQIGEKYGLKMTPADKSDTFKDLDLSTATFLKRGFK
PDSKHPFLVHPIYPIQDIYESIRWTKNPRCLQEHVLSLAHLCWHS
GPEQYADFVKRIRSTSVGKNLYIPSYDVLLYEWYEKF*VNIYSYY
LGSLGV

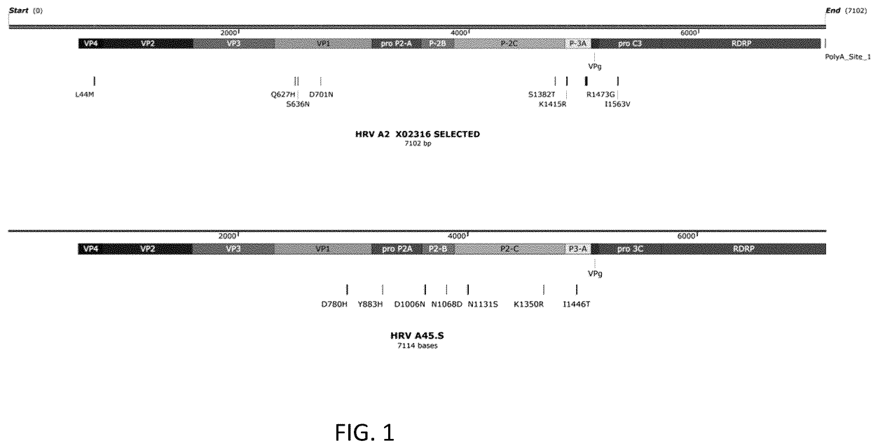

Below is a diagram (FIG. 1) showing amino acid changes within distinct viral polypeptides for HRVA2.s and HRVA45.s. VP1-4 encode the <30 nM structural capsid of each HRV while the remaining subunits encode nonstructural and enzymatic functions. Proteases P2-A and C3 play an important role in cleavage of viral polyproteins, assembly of viral RNA replication, and abrogation of cellular translation. The RNA-dependent RNA polymerase (RDRP) is necessary for viral RNA replication. Although not well characterized, P-2B polyprotein is vital for viral replication and release. P-2C polyprotein, a RNA helicase and ATPase is necessary for viral synthesis. P-3A polyprotein function is the least established, but it is involved in inhibiting intracellular transport. VPg is covalently bound to the 5′ end of the + sense, single strand RNA viral genome and is required for priming RNA synthesis. The vast majority of accrued alterations are in the non-structural coding sequence of each HRV, suggesting that acquired enhanced viral replication may be due to one or more of these selected mutations.

### B. Example 2—Treating Papilloma

Certain human papillomaviruses (HPVs) cause warts. Most warts are benign, though they can still cause discomfort, insecurity, and/or pain for the patient. “High risk” HPV types can become cancer. HPV hides from the host immune system and creates reservoirs of its own DNA within infected cells' nuclei which makes warts particularly difficult to treat.

Infection with HPV is especially tricky to mimic in vitro because it relies on the cell cycle, but studies are ongoing (early pre-clinical phase).

The “solution” to the problem of warts being difficult to treat was developed as an oncolytic virus. Using a wide variety of virus families and cancer cell lines, a virus was created that is tuned to infecting cells with broken cell cycles. The virus that was most prolific within cancer cells is the one being used as a wart therapeutic. Both cell types (cancer and HPV infected cells) exhibit abnormal cell cycles and evade the immune system.

In cancer cell culture, the disclosed viruses can kill quite effectively, but in normal cells, very few are killed.

The disclosed rhinoviruses, unlike HPV, do not care about hiding from the host immune system and stimulates an immune response within the wart/tumor. The virus used causes extremely minor symptoms in humans if infecting the exact environment it needs, so it's very safe, especially as an injectable. The virus is also capable of being lyophilized (freeze-dried under vacuum). Thus, disclosed herein are an injectable and lyophilized viral therapy for warts.

Confirming expression of ICAM1 (HRVA45) and VLDLR (HRVA2) receptors on normal keratinocytes as well as those expressing HPV E6/E7 genes and cell infected with HPV1. Normal human epidermal keratinocytes (nHEK), immortalized/non-transformed keratinocytes (HaCat cells) as well as control HPV driven cancer cells (HeLa cells) have been obtained. ICAM1 and VLDLR expression can be compared in the cells prior to and following expression of E6/E7 or infection with HPV1. In the literature it is reported that ICAM1 expression increases following HPV infection and that LDLR family receptors are expressed in less differentiated nHEKs. Western blots can evaluate receptor expression-data indicates they all express these receptors prior to HPV infection, but this can be assessed any way.

Assessing HRVA2 and HRVA45 infection and replication in nHEK and HaCat cells prior to and following HPV E6/E7 expression or HPV1 infection is performed. Previously, it was shown that these HRVs can infect and kill normal human melanocytes at high MOI in vitro, but these viruses cannot replicate and spread in these cells and within one week uninfected cells proliferate to confluence and there is no detection of HRV RNA. In contrast, infection of human melanoma cells leads to rapid replication and complete cell death in most cell lines. This is due to intact innate immunity (e.g., Toll-like receptors, TLRs, etc.) within each normal cell with limited retention of some innate pathways in the melanoma cell lines. HPV infected cells also suppress innate immune pathways present within normal keratinocytes in order to evade the immune system potentially making them more susceptible to HRV infection and replication.

HPV E6/E7 and HPV1 can be delivered to nHEKs and HaCat cells and assessed for the ability of HRVs to infect and replicate relative to negative (untransfected) and positive HeLa cells in vitro. HPV infected or even E6/E7 expressing cells can be more susceptible to HRV infection and spread.

The impact of cytokine production can also be assessed in a control, HPV infected, and E6/E7 expressing nHEKs and HaCat cells prior to and following HRV infection. It has been shown that a robust pro-inflammatory IFN response ensues following HRV infection of human cancer cells even in the absence of viral replication (normal melanocytes). The IFN response can be seen following HRV infection regardless of HPV suppression.

Any of the disclosed HRVs can be used in the HPV studies. The HRVs can be produced following the same protocol described in Example 1.

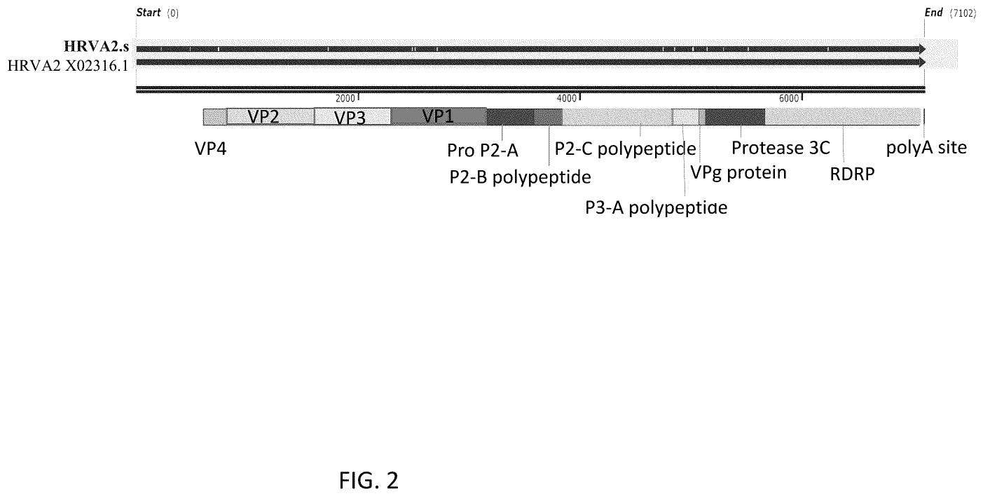

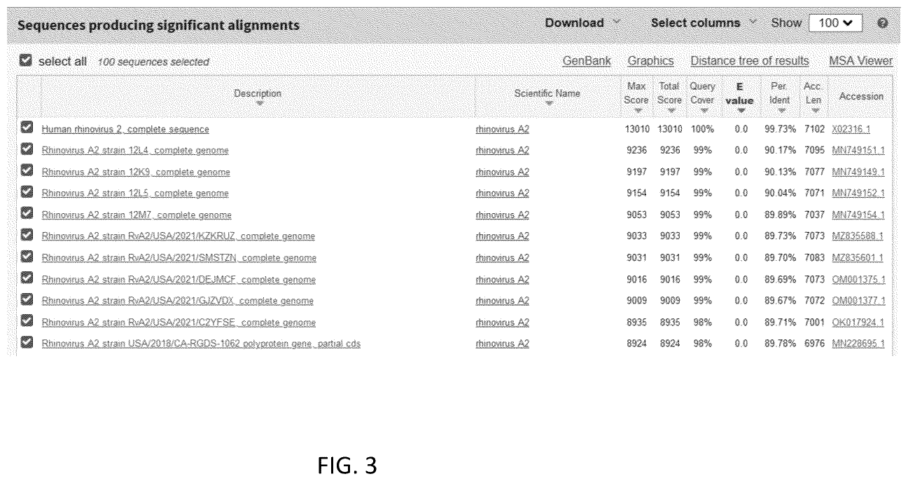

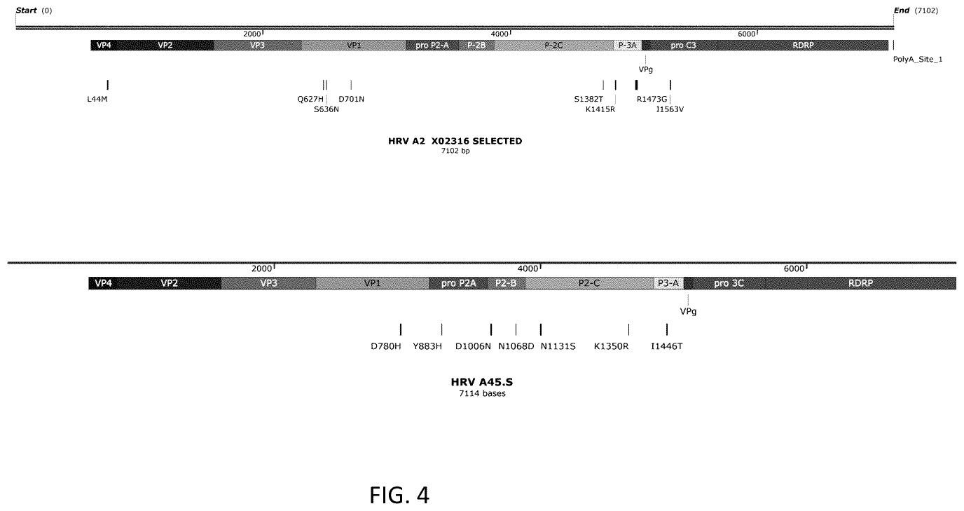

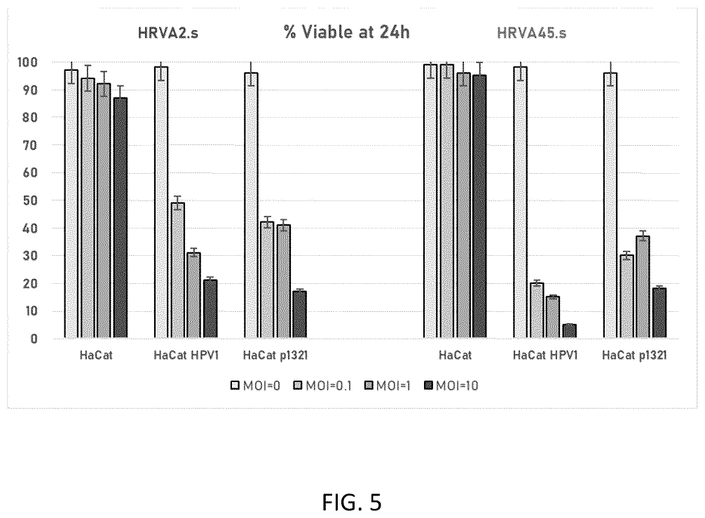

Data showing the ability of the HRVs to preferentially infect human keratinocytes harboring HPV1 or expressing HPV E6/E7 cDNAs in vitro is shown in FIGS. 2-5.

### C. Example 3: Human Rhinoviruses: A Novel Class of Oncolytic Virus

The idea of utilizing viruses to combat cancer has been around for over a hundred years, yet in practice has seen moderate therapeutic success following decades of strategic design to improve tumor selectivity, safety, and effectiveness. It is clear that engaging an immune response is critical to have lasting tumor regression upon treatment, but many patients are unable to mount a sufficient response to standard-of-care immunotherapy alone. Oncolytic viruses (OVs) have been shown to synergize with such therapies to accomplish this goal to varying degrees. The purpose of this study is to investigate Human Rhinoviruses (HRVs) to establish their efficacy as a novel class of anti-cancer agents. We aim to use HRVs to promote longer lasting and more effective immune responses to tumors by fostering immune evasion and viral persistence, and by developing multivalent treatment strategies that enhance tumor regression by preventing tumor escape from viral infection.

In continuing to develop new and improved OVs, researchers have been vigilant in optimizing their ability to selectively infect and replicate within tumors and induce an immune response while striving to minimize toxicity. One key limitation is that modifications of viruses often negatively impact their ability to infect, propagate, and spread within the tumor. Furthermore, induction of strong immune responses also promotes rapid viral clearance. Wild-type HRVs as a novel class of OV was investigated. Multiple HRV serotypes were propagated in human melanoma cell lines to produce highly oncolytic populations of virus. A large panel of cancer types were infected and cytotoxicity was evaluated using flow cytometry and real time live imaging. Pro-inflammatory signaling was assessed by cytokine multiplexing. Tumor responses to HRV were assessed in human xenograft and in syngeneic, immune-competent mouse tumor models. HRVs were found to be capable of infecting and killing a wide variety of human cancer cell lines in vitro and in vivo induce pro-inflammatory responses and tumor regression.

### 1. Introduction

Human rhinoviruses (HRVs) are members of the picornavirus family and are the most common infectious agent known to man. They are the major cause of the “common cold” via infection of the upper respiratory tract. In fact HRVs are estimated to be responsible for up to half of all human infections. Despite this impressive pathogenicity, in a study of short-term repeated HRV exposure most (82%) but not all human subjects infected with high titers of HRV caught colds, even when exposed directly through the upper respiratory tract. Symptoms from infection tend to be mild and HRVs are eventually cleared from the body, though it has proven incredibly challenging if not impossible to generate effective vaccines. This is due to the dynamic evolution of HRV, which occurs via genomic recombination and low fidelity replication (6.9×10⁻⁵ substitutions/nucleotide/cell infection) that generates an incredible amount of diversity. While neutralizing antibodies to HRVs are detected upon infection, antibody titers rapidly decrease over time and are not retained in the body long term, unlike what is seen during infection by other picornaviruses such as coxsackievirus and poliovirus. There is also little cross-reactivity between antibodies against one serotype of HRV versus another. Taking these factors into consideration, HRVs appear to be reasonably safe and have inherent characteristics that may facilitate their persistence in tumors where they can establish a productive infection. While effective OVs need to induce a strong immune response, it may be equally important to give the virus ample time to replicate and spread to enhance the anti-tumor effect. HRVs appear able to meet these criteria in that they are capable of inducing adaptive immunity but are able to evade neutralization through use of multiple serotypes and by avoiding induction of long term memory responses.

In this study human rhinoviruses were sought to be investigated and establish their efficacy as a novel class of oncolytic agent. It was found that HRVs are capable of killing a wide variety of cancer cell types and can replicate to produce a productive infection. HRV infections were demonstrated to stimulate a pro-inflammatory immune response. It was also shown that viral infection promotes oncolysis and adaptive immunity leading to regression in murine tumor models. As such, the data shows that HRVs represent a class of natural oncolytic viruses that are reasonably safe and effective at killing tumor cells while driving anti-tumor immunity, and should be considered for further development in anticipation of clinical testing.

### 2. Materials and Methods

2.1 Cell culture and cell lines Normal human epithelial melanocytes (NHEM) were purchased from Invitrogen. We have previously described the following human melanoma cell lines: A375, C32, C8161R, CACL, LOX IMVI, M14-MEL, MALME-3M, SK-MEL-28, SK-MEL-5, SK-MEL-2, SK-MEL-103, SK-MEL-147 UACC-62, UACC-257. CHL-1 and HeLa-H1 cells were purchased from American Type Culture Collection (ATCC, Manassas, VA, USA). Yale University Mouse Melanoma Exposed to Radiation 1.7 (YUMMER 1.7) cells were purchased from Millipore Sigma (Burlington, MA, USA). Human melanoma cell lines were cultured in RPMI 1640 supplemented with 7% fetal bovine serum (FBS) and gentamicin. YUMMER 1.7 cells were cultured in DMEM/F12 supplemented with 10% FBS, non-essential amino acids (NEAA), and gentamicin. NHEM cells were cultures in 254 media supplemented with human melanoma growth supplement, 10% FBS, NEAA, and gentamicin. Cells were cultured in humidified incubators at 37° C. with 5% CO₂.

2.2 Western blotting Cell lysates were prepared by washing cells once in phosphate buffered saline (PBS) followed by the addition of 0.5 ml of 0.25% Trypsin to remove cells from the tissue culture plates. Cells were collected in RPMI media (7% FBS; gentamicin) washed in PBS, and re-suspended in cold RIPA buffer with protease inhibitors and EDTA for protein extraction. Cell were lysed by agitation on a rotating platform at 4° C. for 20 minutes and clarified at 17,000×g for 10 minutes at 4° C. Protein was quantified using a bicinchoninic acid (BCA) protein assay (Pierce; 23225) according to manufacturer's instructions. Following quantification, 10 μg of protein were diluted in 4× lithium dodecyl sulfate (LDS) buffer (Life Technologies; NP0008) with dithiothreitol, denatured at 95° C. for 5 minutes, and loaded on to 4-12% Bis-Tris gels (Life Technologies; NP0321BOX/NP0323BOX). Gels were run at 200 volts for 40 minutes. Proteins were transferred in NuPAGE transfer buffer (Thermo Fisher; NP0006) to a nitrocellulose membrane (Bio-Rad; 162-0232) at 90 volts for 90 minutes and blocked in 5% non-fat dry milk in 0.05% TBS-T for 30 minutes at RT. Membranes were washed several times in 0.05% TBS-T, and probed with primary antibodies diluted in 5% BSA with agitation at 4° C. The membrane was washed several times in 0.05% TBS-T. Anti-mouse (Cell Signaling Technologies; 7076S) and anti-rabbit (Cell Signaling Technologies; 7074S) secondary antibodies were diluted 1:1000 in 0.05% TBS-T and used to probe the membrane for 1-2 hours with agitation at 4° C. The membrane was then washed several times in 0.05% TBS-T and proteins were detected using enhanced chemiluminescence (ECL) reagent (GE Healthcare; RPN2106). Antibodies used include ICAM-1 (Cell Signaling Technologies; Rabbit; 4915), HA (Cell Signaling Technologies; Rabbit; 3274), and GAPDH (Millipore; Mouse; MAB374).

2.3 Reverse-transcription polymerase chain reaction (RT-PCR) RT-PCR to detect HRV was carried out in two steps. Reverse transcription was performed on RNA samples using the ProtoScript II first strand cDNA synthesis kit (New England BioLabs; E6560S) with HRV serotype-specific reverse primers (Rev 5′-3′). The reaction was carried out at 25° C. for 5 minutes, then 42° C. for 1 hour followed by inactivation of the enzyme at 80° C. for 5 minutes. Once cDNAs were generated for each sample, HRV amplicons (408 bp) were generated by polymerase chain reactions (PCR) using EconoTaq PLUS Green 2X Master Mix (Lucigen; 30033-1) and HRV serotype-specific primers (Fwd 5′-3′; Rev 5′-3′). The PCR was run with an initial 95° C. denaturing step for 10 minutes, followed by 21 cycles of denaturing at 95° C. for 20 seconds, annealing at 55° C. for 20 seconds, and elongation at 68° C. for 30 seconds. The PCR was concluded with a final elongation step of 72° C. for 5 minutes prior to cool down to 4° C. Amplified DNA was detected by running 25 μl of the PCR product on a 1% agarose gel stained with ethidium bromide and imaged with a UV illuminator.

2.4 HRV propagation, purification, and titration HRVs were propagated in HeLa-H1 cells (ATCC). A 10 cm dish of HeLa-H1 cells was infected at 90-100% confluence with 1×10⁶ IFU (MOI 0.1) for 8-24 hours, whereupon cells were observed for signs of cytopathological effect (CPE). Upon confirmation of <50% CPE, cells were scraped into the culture media, collected into conical tubes, and flash frozen using a methanol-dry ice bath. Virus was released from infected cells by thawing in a 47° C. water bath followed by rigorous vortexing for 30 seconds. Freeze-thaw cycles were carried out three times on samples before being cleared at 5,000 rpm for 10 minutes at 4° C. Viral supernatants were then 0.8 μm filtered into clean tubes. Viral stocks were stored at −80 C in aliquots.

To generate purified viral stocks, HeLa-H1 cells in suspension culture were allowed to grow between 5×10⁵ and 1.5×10⁶ cells/mL in 800 mL of S-MEM media [10% FBS, Non-essential amino acids (NEAA), Pleuronic-F68, L-glutamine, gentamicin]. Cells were then pelleted at 500×g for 10 minutes, re-suspended in 45 mL of media, and infected with 35 mL of propagated HRV stock. The infection was carried out at 37° C. for 1 hour with shaking at 70 rpm. 80 mL of fresh S-MEM was then added to the flask and the infection proceeded at 37° C. with shaking at 120 rpm until limited (<50%) CPE was observed. The cell suspension was freeze-thawed and cleared as previously described and aliquoted equally into 25×89 mm SW 28 ultracentrifuge tubes (Beckman Coulter; 326823). 1.5 mL of a buffered 30% sucrose solution (20 mM Tris, 1M NaCl) was added to the bottom of each tube to create a sucrose cushion. Tubes were weighed in rotor buckets and balanced with PBS. HRV cultures were ultracentrifuged at 25,000 rpm for 4 hours at 4° C. Supernatant was decanted and the virus pellets were suspended in 100 μl of PBS, covered, and placed at 4° C. overnight to allow the pellets to dissolve. The pellets were then combined, suspended in 4 mL of PBS, and stored at −80° C. in 50 μl aliquots.

To determine viral titer, HeLa-H1 cells were seeded in two 6-well cell culture dishes at 50-60% confluence. A ten-fold serial dilution of the viral stock was prepared in Opti-MEM media and cells were infected with 500 μl of each dilution for 2 hours at 37° C. Following the infection, cells were washed in PBS to remove unincorporated virus and then incubated in 1 mL of normal culture media (RPMI; 7% FBS, gentamicin) for 72 hours at 37° C. RNA was then purified from the cells by TRIzol (ThermoFisher Scientific; 15596018) extraction according to manufacturer's instructions. Cells were washed in PBS and 400 μl of TRIzol reagent was added to each well. Wells were rinsed vigorously with the TRIzol and cells were collected into tubes and incubated at RT for 5 minutes. 80 μl of chloroform was then added and samples incubated for an additional 3 minutes. Separation of the aqueous, inorganic, and organic layers was carried out by centrifugation at 12,000×g for 15 minutes at 4° C. The aqueous phase was then transferred to a tube containing 15 μg of GlycoBlue (ThermoFisher Scientific; AM9515). RNA was precipitated with 200 μl of isopropanol and incubated for 10 minutes at room temperature prior to being cleared at 12,000×g for 10 minutes at 4° C. The supernatant was discarded and the pellet was re-suspended in 75% ethanol and vortexed prior to centrifugation at 7,500×g for 5 minutes at 4° C. The supernatant was removed and the pellet was allowed to air-dry at room temperature for 10 minutes. Samples were re-suspended in 20 μl of RNase-free water and heated at 55° C. for 15 minutes. RT-PCR was then performed to detect HRVs in the extracted RNA. RNAs were stored at −80° C.

2.5 Flow cytometry Cell death assays were performed using the Attune Nxt Acoustic Focusing Cytometer (Life Technologies). Cells were seeded in 6-well plates at a density of 1×10⁶ cells per well. The following day cells were infected with HRVs at an MOI of 0, 0.1, 1.0, or 10 in phenol red-free RPMI (7% FBS; gentamicin) for 1 hour. Cells were then washed with PBS, re-fed fresh media, and incubated for 24 to 48 hours. At the experimental endpoint media was collected and combined with a PBS wash for each sample. Following treatment with 0.25% trypsin, adherent cells were re-suspended in the media/PBS mixture from each well. Cells were pelleted by centrifugation at 500×g for 10 minutes at 4° C. and re-suspended in 1 ml of 0.2 μM SYTOX Green (Invitrogen; S7020) reagent diluted 1:1000 in PBS from a 5 mM stock. Cells were stained in the dark at room temperature (RT) for 20 minutes prior to being stored on ice and run on the Attune. Cells were detected using a blue laser (488 nm excitation BL1 530/30 bandpass filter) and analyzed for uptake of the dye indicative of cell death. Cell death was quantitated by averaging the percentage of cell death in three replicates. Error bars were generated based on the calculated standard deviation of the replicates.

Tumor profiling was performed using the BD LSRFortessa flow cytometer. Tumor initiation treatments were performed as described. At the experimental endpoint, mice were euthanized and tumors were collected into pre-weighed tubes containing 5 mL of serum-free RPMI media. Samples were then weighed again to determine the mass of each tumor collected. Tumors were dissociated in the RPMI using scissors and forceps and by grinding the tumors between frosted glass slides. 250 μl of 20× Collagenase IV/DNAse was then added and dissociated tumor sample were incubated with gentle shaking at 37° C. for 45 minutes. Following the collagenase digestion, samples were 40 μm filtered followed by an additional wash of 5 ml of scrum-free RPMI into the same collection tube. Cells were collected by centrifugation at 1500 rpm for 5 minutes at 4° C. and decanted prior to resuspension in 1 mL of ACK (Ammonium-Chloride-Potassium; pH 7.22) buffer. Cells were again cleared by centrifugation as before and resuspended in 300 μl of RPMI (2% FBS) and 3 wells/mouse were plated in round-bottomed 96-well plates at 100 μl/well. Cells were pelleted in the plate at 2000 rpm for 1 minute and stained using three different panels, including myeloid [F480 (APC), CD45 (AF488), CD86 (BV421), Ly6C (BV510), Live/Dead Aqua (BV605), CD80 (BV650), CD11b (BV711), MHCII (IA/IE) (BV786), B220 (PE), CD11c (PE-CF594), CD8a (PECy7), Fc Block], lymphoid [CD44 (APCe780), CD45 (AF488), CD127 (PerCP Cy5.5), Live/Dcad Aqua (BV510), PD-1 (BV605), CD4 (BV650), KLRG1 (BV711), CD8a (BV786), CXCR6 (PE), CD62L (PE-CF594)|, and transcription Factor panels [FoxP3 (APC), BCL6 (APC Cy7), CD45 (AF488), GrzB (BV421), Live/Dead Aqua (BV510), Tbet (BV605), CD4 (BV650), CD8a (BV786), TCF1 (PE)].

Staining for myeloid and lymphoid panels was carried out on ice in the dark for 30 minutes and clarified at 2000 rpm for 1 minute. Stained samples were then washed three times in 1×PBS and clarified under the same conditions. Cells were stained again with live/dead stain (1:1000 in PBS) for 10 minutes in the dark at RT. Following live/dead staining, samples were washed three times in FACS buffer and clarified as before. Myeloid and Lymphoid panels were then fixed in Cytofix/Cytoperm for 5 minutes at RT in the dark, washed three times in FACS buffer, and resuspended in 200 μl of FACS buffer prior to overnight incubation at 4° C. in the dark.

Cells used for the transcription factor panel were permeabilized in FOXP3 perm buffer (eBiosciences) and washed in FOXP3 perm wash (eBiosciences). They were fixed in FOXP3 Fix buffer for 10 minutes at RT in the dark followed by additional washes in FOXP3 perm buffer. Antibodies for the transcription factor panel were suspended in FOXP3 perm buffer and cells were stained on ice for 30 minutes. Cells were then washed once in perm buffer and twice in FACS buffer, followed by a final resuspension in FACS buffer and incubated at 4° C. overnight in the dark.

Data was analyzed using a two-way ANOVA test with a Tukey post-test. P values <0.005 were considered significant (P<0.005*, <0.001**, <0.0001***, <0.00001****).

2.6 HRV enzyme linked immunosorbent assay (ELISA) A 96-well ELISA plate (Costar; 3590) was coated with HRV by incubating each well in 2×10⁵ IFU of HRV in 100 μl of PBS for 1 hour at RT. Coated plates were stored overnight at 4° C. HRV was discarded and wells were incubated in 250 μl of blocking buffer (ThermoFisher; TBS Buffer 28358, 0.5% Tween, 0.5% BSA) for 1 hour at RT. Blocking buffer was then discarded and mouse serum samples were diluted 1:50, 1:500, 1:5000, and 1:50000 in blocking buffer and incubated in the antigen coated wells for 1 hour at RT. The plate was then washed three times in 250 μl of wash buffer (ThermoFisher; TBS Buffer 28358, 0.5% Tween) and incubated in HRP-conjugated goat anti-mouse IgG secondary antibody (ThermoFisher, 31430) diluted 1:5000 in blocking buffer. Wells were incubated in 100 μl of secondary antibody for an hour at RT. Following four additional wash steps, 50 μl of TMB reagent (ThermoFisher; 34028) was added to each well. After 15-30 minutes, 50 μl of stop solution (ThermoFisher; SS04) was added to the plate and the absorbance at 450 nm was quantified using a plate reader.

2.7 Mice and injectables. C57BL6 (BL6) mice were obtained from the Charles River Laboratories (Raleigh, NC, USA). Congenic ICAM-1 mice were generated by crossing C.FVB-Tg(ICAM1)4Grom/J mice (The Jackson Laboratory) with C57BL6 mice to produce heterozygous ICAM-1 transgenic mice. The presence of the transgenic ICAM-1 allele was confirmed by PCR using human ICAM-1 specific primers (Fwd 5′-3′; Rev 5′-3′). Transgenic F1 offspring were then backcrossed to C57BL6 mice. This process of selection and backcrossing was carried out for 7 generations. C57BL6 ICAM-1 mice were injected with 2×10⁶ YUMM 2.1 ICAM-1 cells in order to determine whether they were sufficiently syngeneic to allow tumor growth. High tumor penetrance (90-100%) was observed in this model. C57BL6 ICAM-1 mice were then crossed in order to make the transgene homozygous.

Tumors were initiated in 6-9 week old mice by subcutaneous flank injections of 10×10⁶ YUMMER 1.7 ICAM-1 cells. Prior to injection, cells were washed once in PBS and once in Hank's balanced salt solution (HBSS) before a final suspension in 100 μl of HBSS. Tumor re-challenge experiments were carried out by preparing and injecting cells in the same manner in the opposing flank.

HRV intratumoral injections were administered when tumors reached 100 mm³. Mice received a dose of 2×10⁸ IFU of virus every other day for the first week on days 1, 3, 5, and 8, followed by injections once a week for the next four weeks on days 15, 22, 29, and 36. The αPD-1 antibody (clone RMP1-14; BE0146) and the isotype control antibody (clone 2A3; BE0089) were obtained from Bio X Cell (West Lebanon, NH, USA). αPD-1 and isotype control antibodies were delivered systemically by IP injection on days 1, 3, 5, and 8. Tumors were monitored daily and measured three times a week with electronic calipers until the longest diameter of the tumor reached 2 cm or adverse tumor-related complications required animals to be euthanized. Tumor size was calculated using an elliptical estimation:

V=(L×W²)/2,

where tumor volume (V) is equal to the length (L) multiplied by the square of the width (W) divided by two.

Tumor responses were classified as progressive disease (PD), partial response (PR), stable disease (SD), or complete response (CR) according to RECIST 1.1 criteria. According to this criteria, PD is determined by a 20% increase in the diameter of target lesion (with no CR, PR, or SD documented before increased disease). PR is classified as a 30% decrease in the longest diameter of the target lesion with recurrence, whereas SD is when neither PR nor PD criteria are met from the smallest diameter since treatment started. CR is classified as the disappearance of the target lesion with no recurrence. Survival curves were generated and analyzed using GraphPad Prism 9.0.1. Survival distributions were compared using a Log-rank (Mantel-Cox) test. P values <0.005 were considered significant (P<0.005*, <0.001**, <0.0001***, <0.00001****).

2.8 Cytokine and chemokine multiplexing Cytokine/chemokine multiplexing was performed according to the manufacturer's instructions as described previously. Mouse plasma was collected immediately following euthanasia of animal subjects. Upon sacrifice, 200 μl of blood was collected into tubes coated with 10 μl of 0.25 mM EDTA following cardiac puncture. Blood samples were cleared at 1600×g for 10 minutes at 4° C. Plasma was then transferred to clean tubes and flash frozen in a methanol-dry ice bath. Cell culture supernatants were collected 24 hours post-infection, cleared by centrifugation, and flash frozen in a methanol-dry ice bath. All samples were stored at −80° C. Samples were run using the appropriate ProcartaPlex Multiplex Immunoassay kit (Invitrogen; EPXR360-26092-901; Invitrogen, EPXP420-10200-901). Samples were thawed at 4° C. with agitation, vortexed, and cleared at 1000×g for 10 minutes at 4° C. 25 μl of each sample was then added to a 96-well assay plate and incubated with antibody-conjugated magnetic multiplexing beads for 2 hours at RT on a microplate shaker at 500 rpm. Following a wash step using a magnetic plate washer, captured analytes were then probed under similar conditions with biotin-labeled detection antibodies for 30 minutes and washed prior to treatment with Strepdavidin-PE for another 30 minutes. Following the final wash steps, the beads were re-suspended in reading buffer prior to running the plate on the Luminex MAGPIX platform. Analyte differences between samples were compared using an unpaired, two-tailed t-test with GraphPad Prism software version 9.0.1. P values <0.005 were considered significant (P<0.005*, <0.001**, <0.0001***, <0.00001****).

2.9 IncuCyte live cell imaging and analysis Live cell imaging was performed using the IncuCyte platform. Cells were seeded at a density of 1.4×10⁴ cells per well and given time to adhere to the plate for 4 hours. Following cell seeding, HRV infections were performed for 1 hour, after which cells were washed with PBS and incubated at 37° C. Two pictures of each well were taken every two hours for 72 hours to assess cell death over time. Images were collected and compiled into movies using the IncuCyteZoom2016 software.

### 3. Results

i. HRVs Infect and Rapidly Kill Melanoma Cells

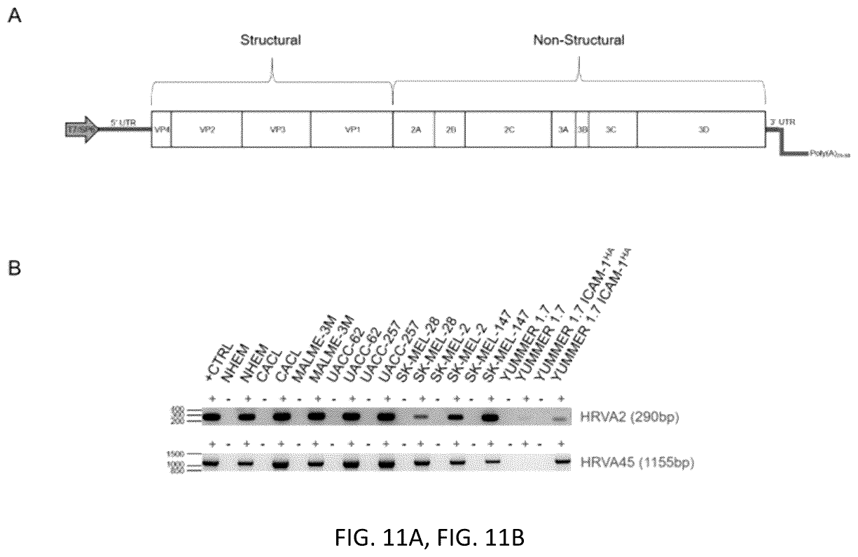

HRVs exploit cell surface receptors for infection, which include ICAM-1 for major group HRVs as shown with CVA21 or LDL-R family members. To test the ability of HRVs to infect melanoma, a panel of cell lines representing BRAF and NRAS-driven variants was infected with HRVA2 and HRVA45 at MOI of 0 and 1.0 for one hour, after which unincorporated virus was washed out and cells were incubated in fresh media at 37° C. for 24 hours. Following another wash, RNA was extracted from cells using TRIzol and the presence of internalized HRV genomic RNAs were assayed by RT-PCR (FIG. 11). Amplicons were not observed in uninfected (MOI 0) cells. Serotype specific amplicons for HRVA2 were detected in each of the other infected cell lines (MOI 1.0), including Normal Human Epithelial Melanocytes (NHEM). Major group HRVA45 was likewise able to infect all cell lines with the exception of YUMMER 1.7/YUMM 2.1 cells, which lack its' cognate receptor. This demonstrates the ability of HRVs to be internalized by melanoma cells.

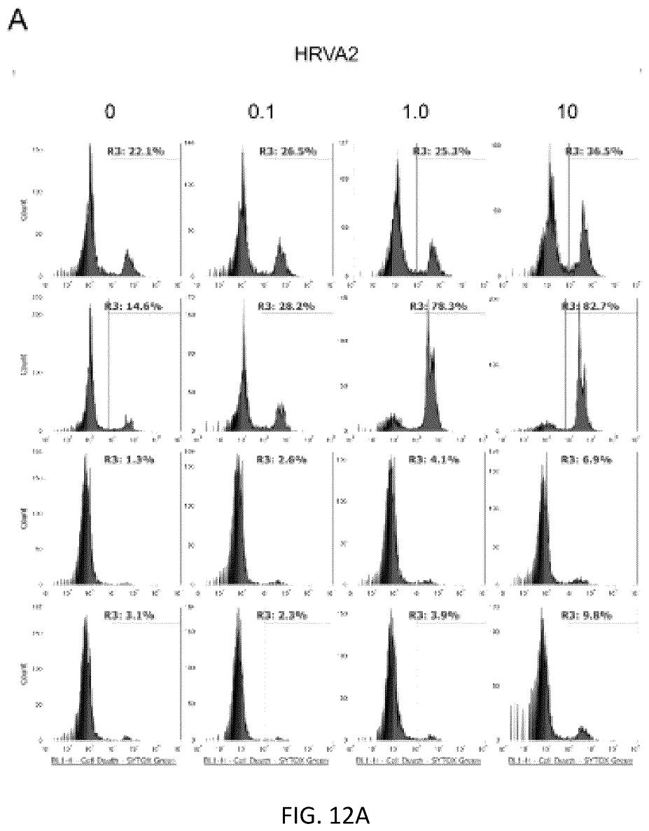

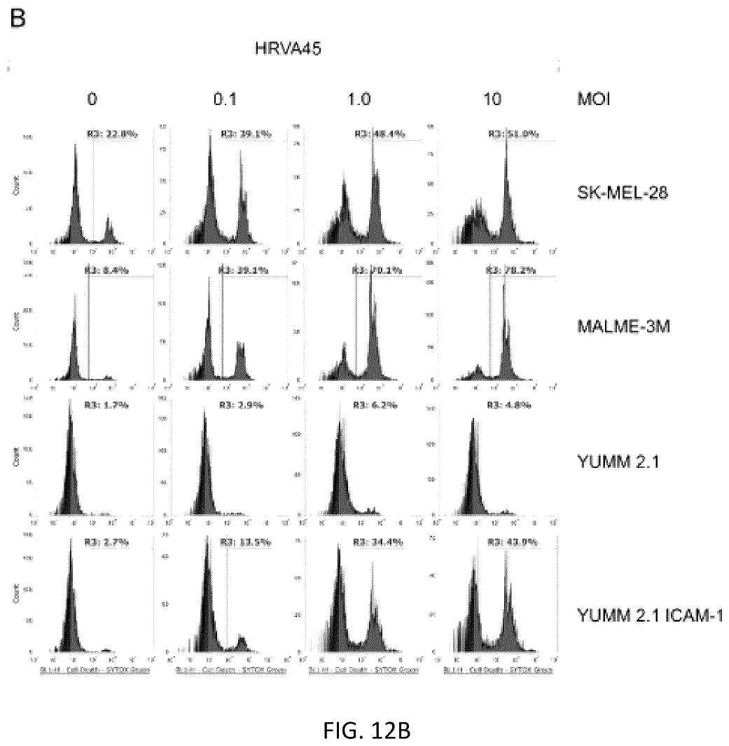

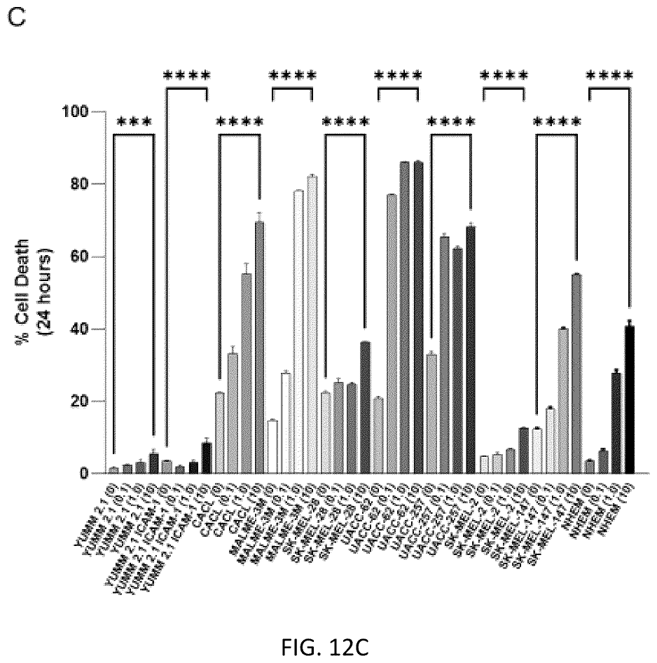

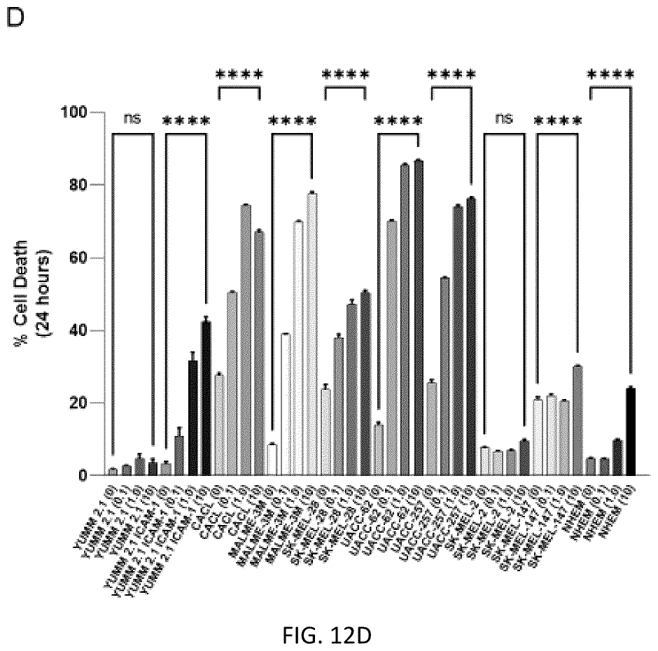

HRVs were then evaluated for their ability to induce cell death upon infection. A panel of melanoma cell lines was infected with HRVA2 and HRVA45 at a MOI of 0, 0.1, 1.0, and 10 for 1 hour, washed to remove virus, and incubated in fresh media for 24 hours. Dead and viable cells were collected from each sample and stained with SytoxGreen prior to analysis by flow cytometry. For most of the melanoma cell lines tested, HRVA2 and HRVA45 induced cell death in a dose dependent manner and these cells were highly sensitive to infection (FIG. 12). The exception was HRVA2 infection of YUMMER 1.7/YUMM.

2.1 and YUMMER 1.7 ICAM-1/YUMM 2.1 ICAM-1 cells, which had been shown to be resistant to infection (FIG. 11B). HRVA45 was able to induce cell death in YUMMER 1.7 ICAM-1 cells, but not in parental YUMMER 1.7 cells. Most susceptible cell lines exhibited high levels of cell death within 24-hours, even at low MOI (FIG. 12). While NHEM cells were sensitive to HRV-mediated cell death, they exhibited some resistance to infection relative to melanoma lines. This was further highlighted by their durability when infections were allowed to go for longer periods (FIG. 12). Thus, when HRVs are incorporated into melanoma cells they induce cell death, demonstrating their oncolytic capability.

qRT-PCR was used to determine how cell death corresponded to viral replication within infected melanoma cells. Infections with HRVA2 and HRVA45 were carried out at a MOI of 1.0 for 0, 8, and 16 hours. For each time point, cell samples and supernatants were collected to assess intracellular and extracellular virus, respectively. Viral RNA was purified by TRIzol extraction as before. Following extraction, samples were analyzed by qRT-PCR. Overall, viral RNA levels appear to be higher in sensitive cell lines relative to resistant cell lines, but this is not always the case.

ii. HRV Infection Induces an Inflammatory Cytokine Response and Production of HRV-Specific Antibodies

As infiltration of immune cells is critical for anti-tumor immune responses, HRV's were evaluated for their ability induce an inflammatory response in infected cells. Melanoma cell lines and NHEMs were infected with HRVA2 or HRVA45 at an MOI of 0, 0.1, and 1.0 for 24 hours, after which cell-free supernatants were collected and probed using a cytokine multiplexing approach. Cytokine levels tended to increase upon infection of NHEMs, resulting in significant increases in [cytokines], though the magnitude of those increases was less than in melanoma cells. There was a significant increase in cytokines in cell lines upon infection with cither HRV, and many additional cytokines that did not attain statistical significance were nevertheless higher in infected cells. Cytokine levels were higher in cells infected with a lower MOI. This is evidence that, whether in melanocytes or tumor cells, HRV infection drives a pro-inflammatory response.

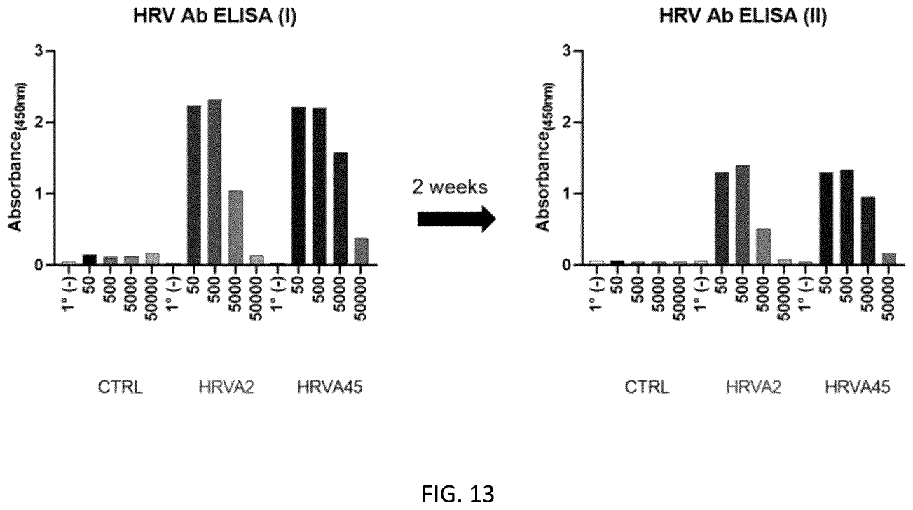

The ability of HRVs to induce an adaptive immune response in a murine host were also assessed. Serum collected from mice inoculated with HRVA2 or HRVA45 was tested against serum of naïve mice for neutralizing antibodies using an ELISA assay (FIG. 13). A 96-well plate was coated with each HRV antigen and incubated with serum samples diluted 1:50, 1:500, 1:5000, and 1:50000. Following antibody capture, wells were washed and treated with anti-mouse IgG detection antibody conjugated to horseradish peroxidase (HRP). Absorbance was quantified using a plate reader. The detection antibody yielded no signal in the absence of serum antibodies, and very little signal was detected in wells treated with the serum of naïve mice. Conversely, wells treated with serum from mice inoculated with either HRV exhibited strong absorbance, characteristic of the binding of virus-specific antibodies. Although this signal was somewhat diminished two weeks later, the presence of HRV antibodies was readily detectable. These antibodies are indicative of an adaptive immune response to HRV infection.

iii. HRV Exposure is Well Tolerated in a Murine Melanoma Model that is Susceptible to HRV Infection of Normal Mouse Tissues

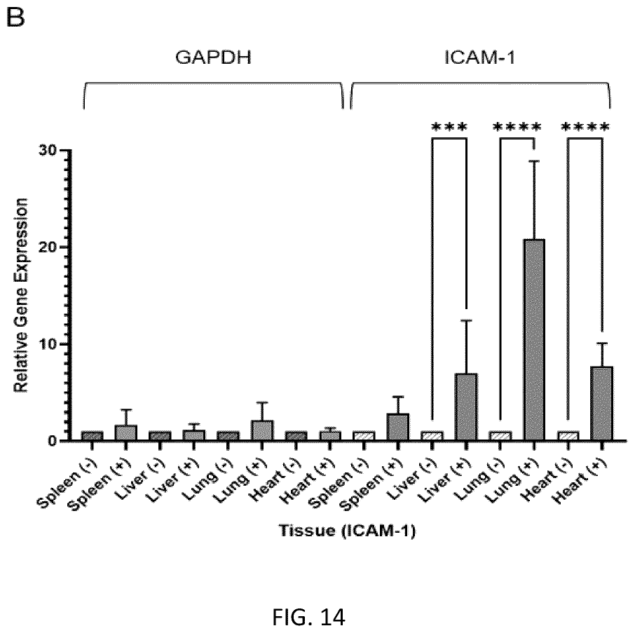

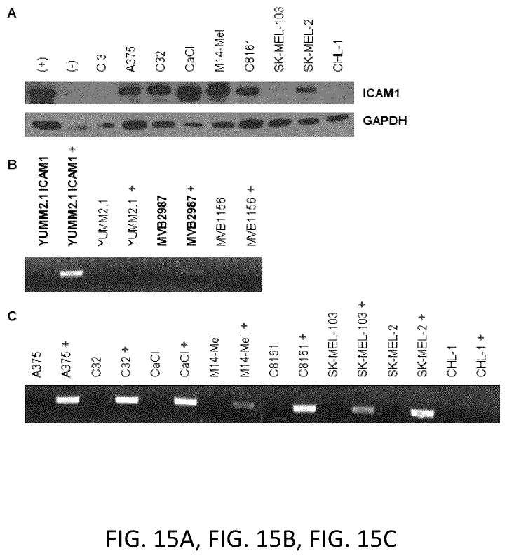

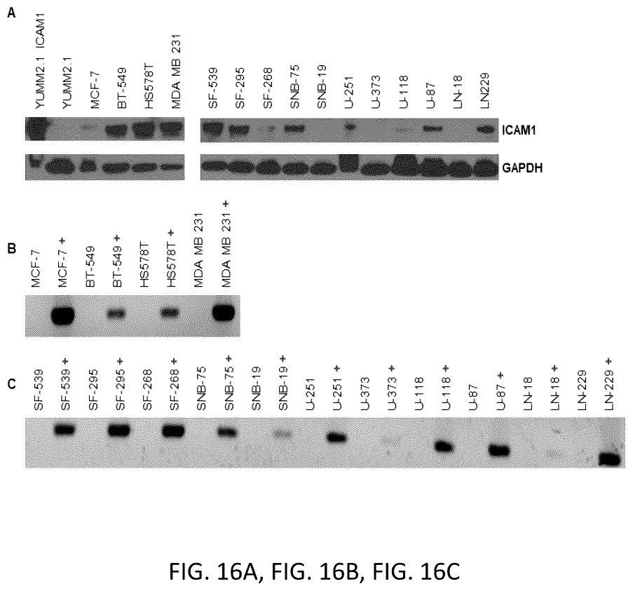

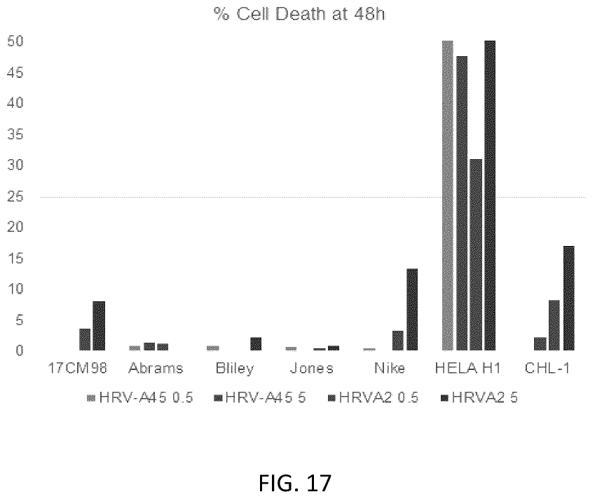

The safety of the virus was then evaluated in vivo. As HRVs are human pathogens and mice are refractory to infection, a murine model was developed to more accurately recapitulate human susceptibility to viral infection. C57BL6 mice were crossed with mice carrying a transgenic ICAM-1 gene under the control of endogenous regulatory elements. Transgenic ICAM-1 mice were backcrossed onto the C57BL6 background until they were sufficiently syngeneic to support growth of YUMMER 1.7 ICAM-1 tumors with high penetrance, after which mice were made homozygous for the transgene. In this model the endogenous expression of ICAM-1 closely mirrors the regulation of ICAM-1 in human tissues, where it is highly prevalent in the lung and spleen and minimally expressed in other tissues such as the kidney, heart, and brain (FIG. 14). Expression of ICAM-1 renders normal murine tissue vulnerable to HRV infection, allowing murine hosts to be used as a surrogate for HRV induced pathogenesis.

The health of C57BL6 ICAM-1 mice was evaluated following systemic delivery of 2×10⁷ IFU of HRVA2 or HRVA45 by intraperitoneal (IP) injection. None of the mice exhibited deleterious symptoms even after repeated dosing in this manner. Similarly, when YUMMER 1.7 ICAM-1 tumors were initiated, repeated dosing of these tumors with intratumoral injection of HRV had no visible effect on mouse health.

To determine how quickly HRVs are cleared from the animals, blood was drawn from naïve mice and those inoculated with 2×10⁷ IFU of HRV by IP injection at 1, 2, 4, and 8 hours post-injection. HRV was detected in the blood using RT-PCR as described previously, where RNA levels were first normalized prior to reverse transcription. HRVs were detected in the blood out to 8 hours following systemic delivery, with viral load decreasing at that time point (FIG. 13C).

Taken together, this data demonstrates that C57BL6 ICAM-1 mice are susceptible to HRV infection but are refractory to HRV-induced pathogenesis. Whether by localized IT injection or systemic exposure to the virus, mouse subjects remain healthy and are capable of clearing the virus relatively rapidly. This safety profile supports further investigation of HRVs for their efficacy as oncolytic agents.

iv. HRVs do not Drive Tumor Regression in an Immuno-Deficient Murine Model of Melanoma

In order to assess the oncolytic activity of HRV independent of the immune system, tumors were generated in immune-compromised nude mice by injecting 2×10⁶ SK-MEL-28 cells subcutaneously in the right flank. In this experiment HRV was tested against CVA21, a known oncolytic picornavirus that has demonstrated efficacy in murine tumor models and clinical benefit (VLA-007 CALM). We have shown that SK-MEL-28 cells are sensitive to infection with both viruses. When tumors reached 50 mm³ they were treated with 2×10⁸ IFU of either CVA21 or HRVA2 on days 1, 3, 5, 8 in the first week and once a week for the next four weeks on days 15, 22, 29, and 36.

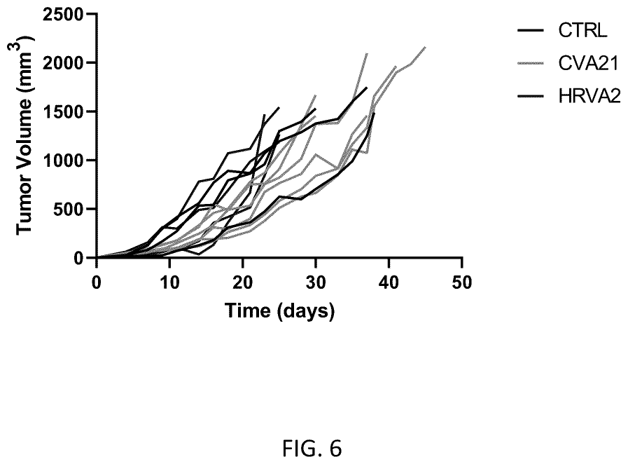

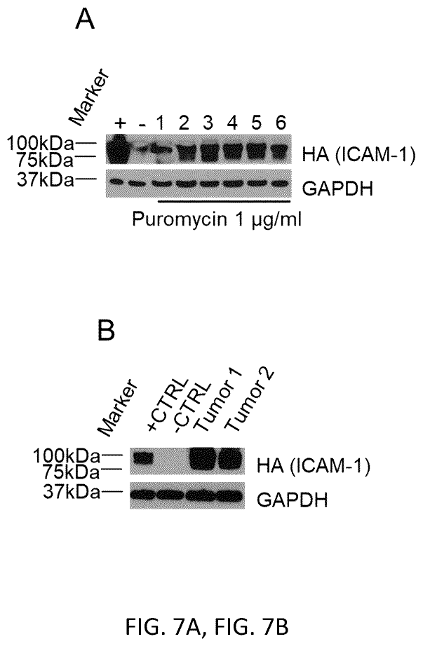

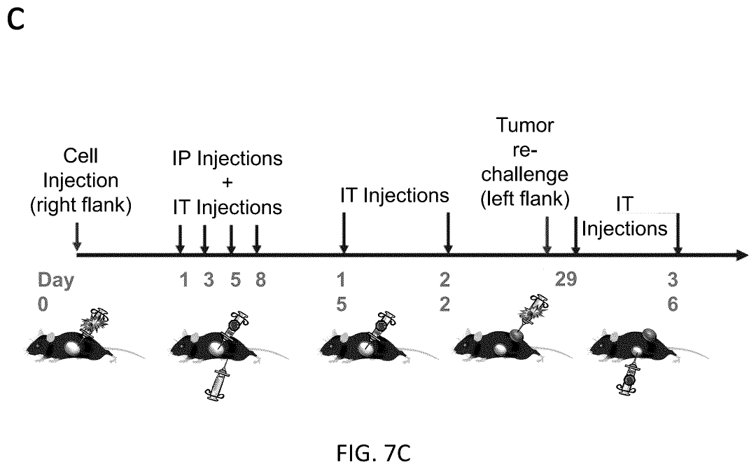

Mice were monitored daily and tumor growth was measured 3 times per week. SK-MEL-28 tumors grew steadily in control mice, with all animals requiring sacrifice due to tumor burden by 40 days (FIG. 6). Similarly, tumors treated with either HRV or CVA21 exhibited little response to viral infection, with mice largely needing to be euthanized within 40 days, and all mice requiring sacrifice due to tumor burden.

v. HRVs Drive Tumor Regression in an Immuno-Competent Murine Model of Melanoma and Responses are Enhanced by Using HRVs in Combination and with αPD-1

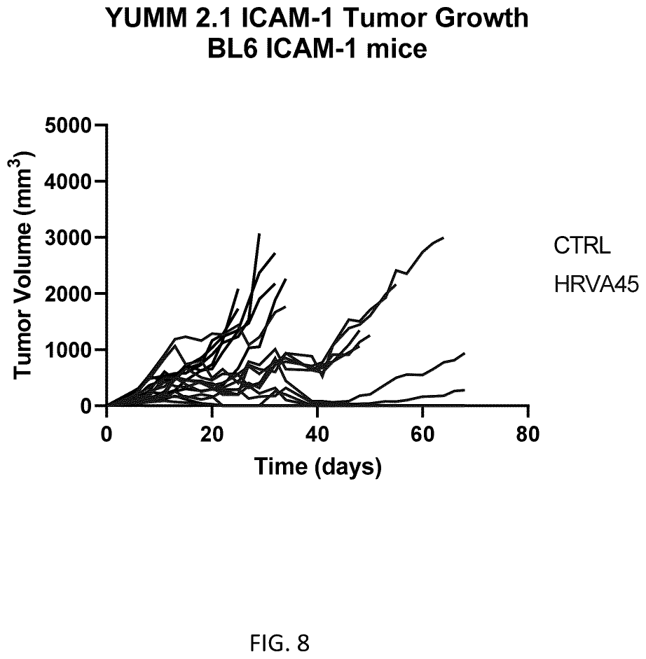

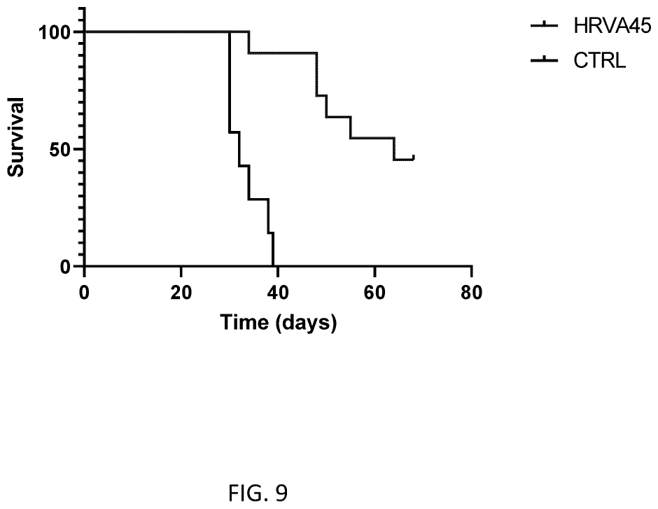

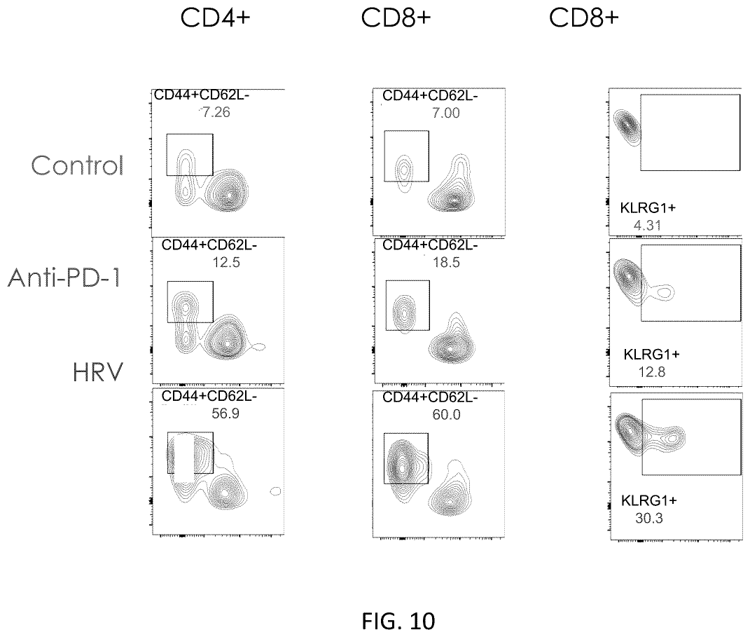

As HRVs demonstrated robust oncolytic activity in vitro, the anti-tumor activity of HRVA45 was then examined in an immuno-competent murine melanoma model. C57BL6 ICAM-1 mice were injected subcutaneously with 1×10⁷ YUMMER 1.7 ICAM-1 cells in the right flank. When tumors reached 100 mm³, mice were given a control saline injection, or 2×10⁷ IFU of HRVA45 via IT injection following the dosing regimen previously described. The experimental endpoint for the study was 70 days (FIGS. 8 and 9).

### D. Example 4

Cancer remains a major public health problem and is the second leading cause of death in the US. In 2019 nearly 1.8 million people will be diagnosed with cancer in the US and over 600,000 will die as a result of this disease. Due to prevention, early detection and improved therapies cancer mortality rates have decreased nearly 1.5% per year since 1991 (27% as of 2016). Several new therapies including immunotherapies have positively impacted mortality in multiple cancer types including melanoma where despite increased incidence, mortality rates have decreased by over 2% each year for the past several years. The historical five year survival rate for advanced metastatic melanoma remained at ˜10% for decades, but recent advances in targeted agents and immunotherapies in particular have dramatically increased this to 23%. Despite these advances, significant innate and/or acquired resistance occurs in the majority of patients treated with single or combined immunotherapies. Therefore, developing novel agents with complimentary function may provide synergy and increase survival rates in melanoma and other cancers.

Immunotherapy has moved to the forefront as a first line treatment of many hematological and solid malignancies. Immune checkpoint inhibitors in particular have proven effective in numerous cancer types. The basic premise of how these agents, typically antibodies, work is through direct blockade of negative signal engagement in T-cells culminating in antitumor activity. Agents that block Cytotoxic T-Lymphocyte Associated Protein 4 (CTLA4) and/or Programmed Cell Death 1 (PD-1) and a growing list of other immunomodulatory receptors and ligands have been approved for the treatment of several cancer types. Additionally, numerous pharmacological and biological agents including oncolytic viruses are being evaluated in combination with a number of these agents.

The application of oncolytic viruses that preferentially target and/or replicate in cancer cells has gained momentum with recent FDA approval of single agent Talimogene laherparepvec (T-VEC) for the treatment of skin and lymph node melanoma in 2015. T-VEC is a modified herpes simplex virus, type 1 (HSV-1, JS-1 strain) that lacks neurovirulence and antigen presentation genes (ΔICP34.5, ΔICP47, U_{L}11 translocation) and encodes two copies of human GM-CSF.⁸ While T-VEC utilizes the same cell surface receptors as wild-type HSV-1 to gain entry in cells, it replicates significantly more efficiently in cells that have disrupted oncogenic signaling (e.g., RAS, BRAF) that lead to suppression of type I IFN and/or PKR signaling and activity. Recently, T-VEC in combination with immune checkpoint blockade (Ipilimumab, Pembrolizumab) has fostered enhanced durable responses in early stage clinical trials prompting further investigation.

As of November 2019 there are 96 clinical trials in the US evaluating an oncolytic virus alone or in combination with immune checkpoint inhibitors, pharmacological, or chemotherapeutic agents. The vast majority of these viruses are attenuated and encode pro-inflammatory factors believed to enhance recruitment and activation of adaptive host immune effectors increasing antitumor activity. Despite improving combined and single agent antitumor activity, rapid viral clearance and effective systemic delivery remain key areas for improvement. In addition, each virus has limited tropism due to reliance on specific cellular receptors in order to gain entry. We have developed novel oncolytic viral agents and strategies for the treatment of cancer.

Human rhinoviruses (HRVs) are among the most common infectious viral agents in humans and are the leading cause of upper respiratory tract infections, including the common cold worldwide. Like all Picornavirus family members HRVs are small (20-30 nM), non-enveloped, and possess non-capped positive-sense single stranded RNA genomes ranging between 7.2-7.8 kb. Seven of the 11 encoded viral proteins (VP) are responsible for replication and assembly while VP1 (mediates specific cell surface receptor engagement), VP2, VP3, and VP4 comprise the icosahedral capsid. There are currently three HRV species known comprising ˜170 serotypes: HRV-A (83), HRV-B (32), and the recently identified HRV-C (55). Nearly 90% of HRV-A/B serotypes, including HRV-A45 utilize intercellular adhesion molecule 1 (ICAM1) as their primary/only receptor while the remainder, including HRV-A2 utilize low-density lipoprotein receptor (LDLR). ICAM1 and LDLR are frequently overexpressed on malignant cells and impact outcome. Cadherin-related family member 3 (CDHR3) appears to be the primary receptor for HRV-C members. The shear breadth of serologically distinct HRVs, rapid induction of escape mutations, frequent HRV coinfection and propensity for recombination has severely limited advances in vaccination.

While there are no reports of HRVs employed as oncolytic agents, two notable Picornaviruses have recently demonstrated significant clinical efficacy. Unmodified Coxsackievirus A21 (CVA21, CAVATAK), which utilizes ICAM1 as its primary receptor, has been assessed in multiple Phase I and II clinical trials in melanoma, prostate, lung and bladder cancers. Local and distant objective responses were observed with intratumoral or intravenous viral delivery with minimal toxicity or adverse events. Addition of immune checkpoint inhibitors (e.g, pembrolizumab, ipilimumab) enhanced clinical benefit PVSRIPO is a genetically modified nonpathogenic poliovirus that is being evaluated in glioblastoma, melanoma, and breast cancer. Although dose limiting toxicities and adverse events were observed in a recent Phase I trial, durable responses rates (21% vs 4% in matched historical controls) were observed out to 36 months in grade IV malignant glioma prompting the FDA to grant Breakthrough and Orphan Status culminating in an ongoing multicenter Phase II trial. PVSRIPO recognizes the poliovirus receptor (CD155) which is expressed on many solid tumors and lymphocytes.

The present invention stems from the finding that significant cell death of cancer cells following incubation with selected HRVs occurs. Limited replication of HRVs is observed in normal cells even when expression of the viral receptor is detected (e.g., ICAM1, LDLR) likely due to intact innate immunity. No active HRV replication is observed in cells lacking expression of the viral receptor(s). No correlation was observed with receptor density and viral replication indicating that at least one receptor is sufficient for viral entry and other factors limit replication.

Six HRV strains were chosen based on receptor dependency (e.g., ICAM1, LDLR) and divergence in sequence similarity. HRV-A2, HRV-A23, and HRV-A25 all utilize LDLR family members while HRV-A45, HRV-B14, and HRV-A73 each require ICAM1 for viral entry. Synthetic near wild-type (wt) genomes of each were loosely based on NCBI accession numbers (Table 1) and have numerous alterations to aid in RNA transcription and further recombinant strategies involving addition of cDNAs. Single base changes include insertions, deletions, and substitutions. Synthetic DNA genomes of each virus were purchased from commercial vendors (GeneWiz, GeneUniversal) and propagated in bacteria by standard means.

TABLE 1
Human Rhinovirus accession numbers

| HRV-A2 | HRV-A23 | HRV-A25 | HRV-A45 | HRV-B14 | HRV-A73 |
|---|---|---|---|---|---|
| X02316 | DQ473497 | MN306047 | FJ445132 | NC001490 | DQ473492 |

Below are the complete synthetic near wt DNA sequences of the sense strand:

HRV-A2
(SEQ ID NO: 5)
TAATACGACTCACTATAGTTAAAACTGGGTCGTGGTTGTTCCCAC
CACGACTATCTACGGATGTAGTGCTCTTGTATTCCGGTACGCTTG
CACGCCAGTTTTGTCACCCCCCCCTACATTGTAACTTAGAAGATT
AACACAAAGACCAATAGGCGGCAATGAACCACATTGTCAACGGTC
AAGCACTTCTGTTTCCCCGGTCAATCTAGATATGCTTTACCAAAG
GCAAAAACTGGAGCGATCGTTATCCGCAAGGTGCCTACGGGAAAC
CCAGTAGCATTCTTTATTTGATCTGGTTGGTTGCTCAGCCGTTAA
ACCCAAACGGTAAACCTGGCAGATGAGGCTGGAAGATCCCCACCA
GCGATGGTGTTCCAGCCTGCGTGGCTGCCTGCACACCCGAAAGGG
TGTGAAGCCTTTTCAAAGACAGGGTGCGAAGAGTCTACTGTGCTC
ACCTTGATTCCTCCGGCCCCTGAATGTGGCTAATCCTAACCCCGT
AGCTGTGGTGTGCAATCCAGCACATTCGCAGTCGTAATGGGTAAC
TGCGGGATGGAACCAACTACTTTGGGTGTCCGTGTTTCTCTTTTT
CCTTTTATATATGCTTATGGTGACAATTATACGGATATAGTTGTC
ATCATGGGCGCTCAAGTCTCAAGGCAGAATGTTGGTACCCACTCT
ACACAAAACACAGTTACAGGAGGATCCAGTCTTAATTATTTTAAT
ATTAATTACTTCAAAGATGCTGCTTCATCTGGAGCGTCAAGGTTG
GACTTTTCTCAAGACCCAAGTAAATTTACAGACCCAGTCAAAGAT
GTTTTAACCAAGGGTATCCCAACCCTACAGTCACCTACAGTAGAA
GCTTGTGGGTATTCAGATAGAATAATACAAATTACCAGAGGAGAC
ACAACAATAACATCCCAGGACATTGCAAACGCTGTAGTTGGATAT
GGAGTTTGGCCTACTTATTTAGATTCAAAGGATGCCTCAGCTATA
GATAAGCCCACACAACCAGATACTTCTGCAAATAGGTTTTACACA
CTGGAAAGTAAGGAATGGACTCCAGATAGTAAAGGATGGTGGTGG
AAATTACCAGATGCACTCAAAGATATGGGTGTTTTTGGTGAAAAC
ATGTTCTACCATGCACTTGGTAGGTCTGGTTACCTAATTCATGTT
CAGTGCAATGCAAGTAAATTTCATTCAGGGACCCTTCTTGTTGTT
GCTATCCCAGAGCACCAGTTGGCATATATAGGCACAGGAAATGTA
ACAGTAGGATACAAACACACCCATCCTGGAGAGACTGGAAGAGTC
ATAGCAACAACCACAGACAAACAGACAAGACAGCCATCGTTTGAC
AGTTGGCTCAATTGTAATGGTACCCTTTTAGGCAATGCCTTAATA
TTTCCACATCAATTTATCAATTTGAGAACAAACAATGCTGCCACC
TTGATCCTGCCGTACGTAAATGCAACTCCCATGGATTCCATGTTA
AGACATAATAATTGGTCATTACTAATAGTGCCTGTATCAGAACTA
AGAGGTGACACCAGTATACCAATTACTGTATCAATCTCTCCCATG
GCAGCTGAATTTTCTGGAGCACGGAACCGCAGTGCACGTGTGGAA
GGTTTACCAGTGATGTTAACACCAGGATCAGGACAATTCTTGACA
ACTGATGACATGCAATCACCATCAGTTTTACCTTACTTCCACTCA
ACACAAGAAATCTTCATACCAGGAGAAGTTAAAAATCTTATTGAA
TTGTGCCAAGTGGACACCATGGTACCATTAAACAACTTGCACATA
AATAAAAACAAGATTGGAATGTATGCTCTACCACTTACACGACAG
AATACACCAGCTGCTGAACTCTTTGCAATGCCAGTCGACATTACA
TCTTCACCTTTAGCAACCACGCTTTTAGGAGAGATTGCTTCTTAT
TACACCAACTGGACAGGTAGTATAAGATTGAGTTTCATGTTTTGT
GGCAGTGCAAACACCTCACTCAAGCTTCTTCTAGCATATACTCCA
CCTGGAGTGAGTAAACCAACCAGTAGGAGAGAAGCTATGCTGGGC
ACCCATCTTGTATGGGATGTAGGTCTTCAATCCACATGTTCTTTA
GTCATCCCGTGGATATCAGCATCACATTTTCGGAATACCACGCCA
GACACCTACTCAAAAGCTGGTTATGTGACATGCTGGTATCAGACA
AATTTCATCACAGCACCAAACACACCACCTACAGCTGACATTATT
TGTCTGGTTTCAGCATGTAAGGATTTTTGCTTACGCATGGCTAGA
GATACTAACCAGCACACTCAGCTTGGAGCAATAGAGCAAAACCCT
GTTGAACAATTTGCAGAAGCAGTCCTTGATCAAGTATTAGTAGTT
CCAAACACTCGACCCAGCGATGGGTTGATTGCAAACTCAGCCCCA
GCTTTGGATGCAGCTGAAACTGGACACACCAGTTCAGTGCAGCCT
GAGGACCTTATAGAGACTAGATATGTGATTGCAGACCAAACCAGA
CATGAAACCTCCATTGAATCTTTTTTGGGTAGGGCTGGATGTGTG
GCCACTATTAGTTTAGACATTAACCATGATGACTACCAAAAGAAT
TACAAAAATTGGGCAATTAGTTTACAAGAAATGTCACAAATTAGG
AGGAAATTTGAAATGTTTACATATGTCAGATTTGATTCCGAAATA
ACAATAGTACCATGTGTTGCTGCCACAGAAGGTAACTTGGGACAC
ATTGTTGTGCAATACATGTTTGTACCACCAGGAGCACCTCTCCCT
GTTAGTAGAACTGACAACACTTGGCAATCTAGCACAAATGCATCA
GTCTTTTGGCAGGTTGGTCAAACTTATCCCAGATTTTCTATACCT
TTCTCAAGTATAGCTTCAGCTTACTACATGTTTTATGATGGATAC
GACACTGATGGCACAGATGCAGTGTATGGTGTTAGTGTGACTAAC
GATATGGGGACTATATGTGTTAGAATTGTTACAGACCAACAACAA
CATAGAGTTAAGATCGACTCCATGGTATATCTAAAAGCTAAACAC
ATCAAGGCATGGTGTCCCAGACCTCCAAGAGCAGTCACATATAAC
CATACATATAATCCAAATTATGTTAGGGCTGATGAAACAGCCACA
AAAGTCCAAACTAGAGCAAATGTCACAACAGTAGGTCCATCAGAC
ATGTTTATTCACGCCTCAGAGTTTTTATACAGAAATTACCATCTC
ACTCCAGAAAAGGAACTGAAAGAAGCATGCCAAATTGTATATACT
GCAGATCTAGTTATACACCGCACAAGAGACAAAGGTGATGACTAT
ATCCCACAATGCAATTGTACAGATTGCTGTTACTACTGTGCCCAC
AAAGACAGGTATATTCCCATCAAAGTAGAATACCATAGCTACTAC
ACCATCCAGAAATCAGATTATTATCCAAAACATATACAGTATGAT
ATACTAATTGGTGAGGGACCTTCGCAACCAGGTGATTGTGGAGGA
AAACTTTTATGCAGACATGGTGTTATTGGTATGGTGACAGCTGGA
GGGGAAGGACATGTAGCATTTACTGATTTGAGGAAGTATAGAATG
GTAGAGGCTGAGGAGCAAGGTATAACAGATTATGTTAAATCCTTA
GGTGATGCTTTTGGAGTAGGATTTGTAGATCAAATTAAAGAACAA
ATTAATAATATAAACCCATTAAATAAAATCAGTGCTAAAGTGATC
AAATGGCTAATCAGAGTAATATCAGCACTAGTGATAGCAGTGCGT
AGCCAAGGGGATCCAGCAACACTATCAGCTACTCTACTTTTACTT
GGGTGCTCTAATTCCCCGTGGCGGTTCCTGAAACAGAAGGTGTGT
ACATGGTTGGGGCTTAGATACATACACAAAGAATCAGATGGTTGG
ATCAAGAAATTTACTGAAATGTGCAATGCAGCCAGAGGCTTAGAA
TGGATAGGGTGTAAGATTTCAAAATTCATTGACTGGCTGAAATCC
ATGCTACCTCAAGCACAGAACAAAATCAAGTTTCTCCACTTCATG
AAACAATTGCAGCTAAAAGAAAAACAGATCGATGGTTTACCTTAT
GCAACAGTTAAGCAACAAGAAGATTATCTCAAAGAAATGGAAGAG
ATGTTGGACATTTCAAATAAACTATTACCACTATACCCAAAGGAG
AATAAGATTATAAAAGATCTACTCAAACAAGCTAAAAGCATGACA
ACAACATCAAAGAGAGTTGAACCAGTTGCAATCATGTTTCATGGA
GATCCAGGGTCAGGGAAATCAGTGTGCACAAACATCCTTGCCCGC
ATGATAACTAATCCATCAGACATATATTCCCTACCCCCAAATCCA
AAGTATTTTGATGGATACCATCAACAGACTGTAGTAATAATGGAT
GATGTGATGCAAAACCCAGATGGGGAAGACATGAGCACCTTCTGC
CAAATGGTTTCCTCAGTTAATTATGTAGTGCCAATGGCTGATCTA
CCAGATAAGGGTACCCTGTTTTCATCAGATTATGTCTTTTGCAGT
ACAAATCAGCATGTTTTGATTCCTCCAACCATATCTACCATACCC
GCCCTGAATCGGCGATTGTTCTTCGATCTAACTGTTAAGGTGAAC
CCCAGATATCAGGAAGCAGGAAAACTAAATCTGGACTGTGCATTA
AAACCATGCAATCATGAGTTAAAGGTTGGAAATGCAAGGTGTTGT
CCTCTCATTTGTGGTAAGGCTATAACCTTCATTAACAGACACAAC
AATGAAGAATTGACACTGTCCCAAATATACAATCAAGTAGTAAAT
GAACACAACCGCAGATTAAACGTGTCAAAACACATGGAAGCAATT
TTCCAGGGACCTATTGATATGCAAGCACCTCCCCCACCAGCAATA
GTTGATTTACTTAGATCCACCAGAAATGAAGATGTCATCAACTAC
TGCAAAAACCAAAATTGGATTATTCCTGCTGACATCAGTATTGAG
AGAGAATTGAATCTGGTAAATCTTTCAATATCAATTCTGGCAAAT
CTAATTAGTGTGATAGGAATCATTTACATTATATATAAATTATTT
GTATCATTACAAGGACCTTATAGTGGACTACCCACCAAGAAAAAA
GTGATTCCAGAGAAAAGAGTGGTAGTACAAGGTCCTTCTACTGAA
TTTGGCCTAAGTTTAATAAAACATAACACATGTATTGTAGAAACA
GAAAATGGTAAGTTTACAGGTTTAGGAATATACGATAACCTATTA
GTCATCCCCACACATGCTGATCCCAGTAGAACTGTCAAGGTGGAT
GGAGTGGAAACAGAAGTGTTGGACTCTTACGATTTGTACAACAAA
GAAGGAGTAAAATTAGAAATCACAGTTCTGATCTTATCCAGAAAT
GAAAAGTTTAGGGATATTAGAAAATACATACCAAATTCTGAGGAT
GATTATGTCAACTGTAATCTGGCTTTAGTTGCAAACCAAGATATG
CCACAGATACTGGAGGTCGGAGATGTTGTCTCTTATGGCAATATA
CTCTTGAGTGGTAATAACACCGCCAGAATGCTCAAATATGAATAC
CCTACAAAATCGGGTTTTTGTGGAGGTGTTTTGTACAAGGTGGGT
CAAGTAATTGGCATACACGTAGGTGGCAATGGAAGACAAGGATTC
TCAGCCATGTTACTTAAAAGATATTTCAACCAACAACAAGGTCAG
ATAATTCTAAAGAAACCAGTCAAGGAAGTTGATTATCCCAGTATA
CATACCCCTACTAAGACAAAACTCCAACCAAGTGTTTTCCATGAT
ATTTTTCCCGGAGTTAAGGAACCTGCAGTTCTAACAGAAAAAGAC
CCCCGGTTGGAAGTAGACTTGAACTCCTCTCTTTTTTCAAAATAT
GCTGGAAATGTTAATTTAGAAATGAATGAGTACATGATTGTTGCA
GCATCCCATTATGCATCACAACTTGAAACACTAGACATATCCAAC
CAGCAAATGTCTATTGAGGAATGTGTGTATGGAACAGATAACTTA
GAAGCCTTGGATTTAAATACTAGTGCAGGATTTCCCTATGTGGCT
TTGGGAATTAAGAAGAAGGACTTAATAAATAGAGAGACTAGAGAT
GTCAGTAAAATTAAAAATTGCTTAGACACTTATGGTGTAGATTTA
CCAATGATAACATATTTAAAAGATGAGCTCAGAACACCAGAAAAA
ATAAAGTTAGGAAAAACTAGGGTAATAGAAGCAAGTAGTTTAAAT
GATACTATTCATATGAGAATGCTCTTTGGAAACTTGTTTAAAGCC
TTTCACGCAAATCCAGGCATAGTAACAGGGTGTGCAGTTGGTTGT
GATCCAGAAACATTTTGGTCTAAAATTCCTCCAATGCTGGGTGAT
GGGTGTGTGATGGCATTTGATTATACAAATTATGATGGAAGTTTA
CATCCAATATGGTTTAGGCTACTTGAAAGAGTGCTGGATAGGTTG
GGATTCCCAGGCTGTGCAGTTAGAAAATTATCACACTCCACCCAT
ATATACAAAGGAATGTACTATGAAGTTGATGGAGGAATGCCATCT
GGGTGTGCAGGTACTTCAATCTTCAACTCAATGATCAATAACATC
ATAATCAGAACAATAATTCTACATGCTTACAAGAATATAGACCTG
GATCAGTTGAGGATACTTACATATGGTGATGATATAATTTTTACT
TACCCAGATAAACTAGACATGGCTTATTTAGCCCAAATTGGAGAA
AAATATGGTTTAAAAATGACACCTGCAGACAAGTCAGATACATTT
AAGGATTTGGATCTGAGTACAGCAACTTTCCTAAAAAGAGGCTTT
AAACCTGACTCAAAACACCCATTTTTAGTCCATCCCATATATCCA
ATCCAGGATATCTATGAATCAATCAGATGGACAAAGAATCCCAGA
TGTTTACAGGAGCATGTGCTCAGTTTGGCTCACTTATGCTGGCAT
AGTGGACCAGAGCAGTATGCTGATTTTGTCAAGAGAATCAGATCA
ACATCTGTTGGGAAGAACCTATACATACCATCTTATGATGTACTA
CTTTATGAGTGGTATGAGAAATTTTAAGTTAATATATACAGTTAC
TATTTAGGTAGTTTGGGTGTATAAAAAAAAAAAAAAAAAAAAAAA
AAAAAAAAAAAAAAAAAAAAAAAAAAAGAAGAGCTTTAGTGAGGG
TTAATT
HRV-A23
(SEQ ID NO: 6)
GCGGCCGCATTTAGGTGACACTATAGCCAAAGTAGTTGGTCCCGT
CCCGCATGCAACTTAGAAGCTTTGCACAAAGACCAATAGCCAGTA
ATCATCCAGATTACTTAAGGTCAAGCACTTCTGTTTCCCCGGTCA
AAGTTGATATGCTCCACCAGGGCAAAAACAACTGCGATCGTTATC
CGCAAGGCGCCTACACAAAGCTTAGTAGCATTCTGAAAGTTGTCT
GGTTGGTCGTTCCACCATTCCCCCTGGTAGACCTGGCAGATGAGG
CTAGAAACTCCCCACTGGCGACAGTGTTCTAGCCTGCGTGGCTGC
CTGCACACCCTCTGGGTGTGAAGCCATACGATGGACAGGGTGTGA
AGAGCCCCGTGTGCTCGCTTTGACGTCCTCCGGCCCCTGAATGTG
GCTAACCTTAACCCTGCAGCTAGAGCACACAATCCAGTGTGTTTC
TAGTCGTAATGAGCAATTGCGGGACGGGACCAACTACTTTGGGTG
TCCGTGTTTCCTTGTTCTCTTTGAATATCTGCTTATGGTGACAAT
ATATACGTATATATTGACACCATGGGCGCACAAGTATCCAGGCAA
AATGTTGGAACTCACTCCACACAGAATTCTGTATCAAATGGATCT
AGTTTGAATTATTTTAATATCAATTACTTCAAGGATGCTGCTTCA
AATGGAGCATCTAAATTGGAGTTTTCACAAGATCCTAGTAAATTT
ACTGATCCTGTTAAAGATGTTTTGGAAAAAGGAATACCAACACTG
CAGTCGCCTACAGTGGAAGCTTGTGGGTACTCTGATAGAATTATA
CAAATTACTAGAGGAGATTCAACAATAACTTCACAAGATGTGGCT
AATGCTGTTGTTGCATACGGTGTTTGGCCGCACTACTTGTCATCT
GAAGACGCCACTGCAATTGACAAACCCTCCCGTCCTGACACATCG
TCCAACAGATTCTACACACTAAAGAGTATAACTTGGTCTAGGTCT
TCAAAGGGTTGGTGGTGGAAACTACCAGATGCATTGAGAGATATG
GGTATCTTTGGTGAAAACATGTTCTACCATTATCTGGGTAGGAGT
GGGTATACAGTGCATGTGCAGTGTAATGCTACTAAATTTCATCAA
GGCACACTGGTGGTTGCAATGATACCTGAACATCAAATCGCTAGT
CTTCAACACGGCGATGTAAATGTCGGGTACAATTTTACCCACCCG
GGAGAGGAAGGGAGAGAGGTCAAAAACACTACACGTGAACATCTT
CAGCAGCAGCCTACTGAAGAACACTGGCTCAATTTTGATGGTACA
TTATTAGGAAATATTACAATATTTCCTCATCAATTTATTAATCTG
AGGAGTAACAATTCTGCCACAATAATTGTCCCGTACGTTAATGCA
GTTCCAATGGATTCAATGCGAAGCCACAATAATTGGAGTTTAGTG
ATAATCCCAATTTGCCCTCTTGAGACAATGAGCACCATCACCACG
GTACCTATTACAATTTCCATTAGTCCAATGTGTGCAGAGTTCTCT
GGTGCACGTGCTAGAAGCCAAGGCTTACCGGTGTTAATTACACCA
GGCTCAGGTCAATTTCTAACAACAGATGACTTTCAATCTCCATGT
GCACTTCCTTGGTACCATCCAACTAAAGAAATTTTCATTCCAGGT
GAAGTCAAAAACTTAGTTGAAATCTGTCAAGTAGATAGCTTGATA
CCAATAAACAACCTTGAAGGGAACATAACTAGTGAAGCTATGTAT
TCAATAGAACTGCAGTCATCGACTGAACAAAGTAAGATATTTTCT
ATTAGAACAGATATTGCTTCCCAACCTTTAGCTACTACTCTAATT
GGTGAAATTTCTAGTTATTTTACACACTGGACAGGGAGTTTACGT
TTCAGTTTTATGTTCTGTGGTACTGCCAATACTACTGTCAAACTT
TTATTAGCATACACACCACCTGGTATTGCAGAACCTACAACAAGG
AAAAATGCAATGTTGGGTACCCATATAATATGGGACATAGGTTTG
CAATCTACAATATCAATGGTAGTACCTTGGATCAGTGCCAGCCAT
TATAGAAACACATCACCAGATGTGTCTACATCTGGATTCATAACT
TGCTGGTATCAAACTGGATTAGTTATTCCACCTGACACTCCTTCC
ACAGCTAGATTATTGTGCTTTGTCTCTGGGTGCAAAGATTTCTGC
TTGCGCATGGCCCGAGACACCAATTTGCACATGCAGAAAGGTATA
ATAGCACAAAATCCAATTGAGAACTATGTAGATGAAGTTCTTAAT
GAAGTCTTAGTTGTTCCCAATATCAATAGTAGTCATCCCACAACA
TCAAACTCTGCTCCAGCATTAGACGCTGCGGAAACGGGTCACACT
AGTAATGTTCAACCAGAAGATGTCATTGAAACCAGGTACGTTCAA
ACATCACAAACAAGAGATGAAATGAGTTTAGAAAGTTTCCTTGGT
AGGTCAGGGTGTATACATGAATCTAAATTAAAAGTTGAGATCGGA
AACTATGATGAAAACAATTTTAATACTTGGAATATTAATTTACAG
GAAATGGCCCAAATCAGAAGAAAGTTTGAACTGTTTACTTACACT
AGATTTGATTCTGAAATTACTTTGGTTCCATGCATTTCTGCTCTT
AGTCAAGATATTGGTCACATCACAATGCAGTATATGTATGTCCCA
CCAGGTGCTCCAATACCGAAAAGTAGAAATGACTATGCATGGCAA
TCTGGAACAAATGCGTCCATTTTTTGGCAACATGGACAAACATAT
CCAAGGTTCTCCTTACCCTTTTTGAGTGTGGCATCTGCTTATTAC
ATGTTTTATGATGGATACAATGAGAAAGGCACGCATTATGGAACA
GTTAGCACAAACAACATGGGCACATTGTGCTCCAGAGTGGTAACA
GAGAAACACATTCATGATATGCGGATAATGACAAGGGTCTACCAC
AAAGCTAAACATGTCAAAGCATGGTGTCCGCGGCCACCCAGAGCA
CTTGAATACACACGCGCTCACCGTACTAATTTCAAAATTGAAGGT
GAAAATGTCAAATCAAGGGTTGCACATAGACCTGCAGTGATAACA
GCTGGCCCCAGTGATATGTATGTACACGTAGGGAACCTCATTTAC
AGAAACCTTCATCTCTTTAATTCTGAGATGCATGAATCTATCTTG
GTGTCTTACTCTTCAGATCTAATCATTTACCGAACAAACACTATA
GGTGATGATTATATCCCTTCTTGTGATTGCACTCAAGCTACATAT
TACTGTAAACACAAAAATAGATATTTTCCGATCACTGTTACAAGC
CATGACTGGTATGAAATACAGGAGAGTGAATATTACCCTAAGCAC
ATACAATACAACTTGTTAATTGGTGAAGGGCCTTGTGAGCCAGGT
GATTGTGGTGGAAAACTACTATGTAAGCATGGAGTTATAGGCATT
GTAACAGCTGGTGGTGATAATCATGTAGCTTTCATTGACCTTAGA
CAATTTCATTGTGCTGATGAGCAAGGAATCACAGACTACATACAC
ATGTTAGGTGAGGCATTTGGAAATGGATTTGTAGATAGTGTGAAG
GAGCATGTGCATGCCATTAACCCAATTGGTAATATTAGCAAGAAA
ATTGTCAAATGGATGTTAAGGATAATATCAGCAATGGTTATAATA
ATTAGAAACTCTTCTGACCCACAAACCATATTGGCAACACTAACA
CTGATTGGATGTTCAGGATCTCCGTGGAGGTTTCTCAAAGAAAAA
TTTTGTAAGTGGACACAGCTTAGTTACATACATAAGGAATCAGAT
TCATGGTTAAAGAAATTCACTGAAGCATGTAATGCAGCAAGAGGT
CTTGAATGGATAGGGAATAAAATATCCAAATTCATTGAGTGGATG
AAGTCAATGCTTCCGCAAGCTCAACTTAAGGTCAAGTACTTGAAT
GAACTTAAGAAATTAAGCCTATATGAAAAACAAGTTGAAAGTCTA
AGGGTGGCAGATATAAAAACCCAGGAGAAAATCAAAATGGAGATT
GATACTTTGCATGACTTGTCATGCAAATTCTTGCCACTGTATGCA
AGTGAAGCAAAAAGAATAAAAACCCTCCACATTAAATGTGATAAT
ATAATTAAACAAAAGAAGAGATGTGAACCAGTAGCTATAGTCATT
CATGGACCACCAGGCGCTGGTAAGTCTATAACAACAAATTTCTTA
GCCAAAATGATCACTAATGACAGTGATATTTATTCCCTTCCTCCA
GACCCAAAATACTTTGACGGTTATGACCAGCAGAGTGTGGTTATA
ATGGATGACATCATGCAAAATCCAACTGGAGATGACATGACTTTA
TTCTGTCAGATGGTATCTAGTGTTACCTTTATACCACCAATGGCT
GATCTGCCAGACAAGGGTAAAGCTTTTGATTCTAGATTTGTTTTG
TGTAGTACAAACCATTCTCTCTTGGCACCACCCACAATAACCTCG
CTCCCTGCTATGAATAGAAGATTCTTTTTTGATCTGGATATAATA
GTACATGATAATTTCAAAGATTCACAGGGAAAACTTAATGTGGCA
GCAGCTTTCAAACCATGTGATGTAGATATCAAAATAGGAAATGCA
CGCTGTTGTCCATTTGTGTGTGGAAAAGCAGTCTCCTTCAAAGAT
CGCAATTCTTGTAATAAGTATACCTTAGCACAAATTTATAACATA
ATGCTTGAAGAGGATCGGCGCAGAAGACAAATGGTTGACGTCATG
TCAGCTATATTCCAGGGGCCAATTGACATGAAAAATCCACCACCA
CCTGCCATCACTGATTTACTCCAATCCGTTAGAACCCCCGAGGTT
ATTAAATATTGTGAAGAAAACAGATGGATAATCCCAGCAGAGTGT
AAAATAGAAAAAGAGCTAAACTTGGCTAATACAATTATAACAATT
ATTGCTAATGTTATTAGTATAGCAGGTATTATATATGTGATTTAT
AAATTGTTCTGCACACTACAAGGACCTTACTCAGGGGAACCAAAA
ATCAAAACTAAGATTCCAGAGAGGCGTGTGGTGGCACAAGGACCA
GAGGGGGAGTTTGGAATGTCTCTGATCAAACATAACTCGTGTGTT
GTCACAACAGAAAATGGCAAATTTACAGGCCTTGGGATATATGAT
AGAACTTTGATTGTGCCAACCCATGCTGATCCTGGAAAGGAAATC
CAAATTGATGGCATAACAACAAAAGTGATTGATTCATATCACCTA
TATAACAAGGATGGTATTAAACTTGAGATAACAGTTCTAAAGTTA
GATAGGAATGAGAAGTTTAGAGATATTAGAAAGTATATACCAAAC
AATGAAGACGACTATCCAGATTGTAACTTGGCCCTATTGGCAAAC
CAACCTGAACCCACTATAATTAATGTTGGTGATGTTGTATCCTAT
GGTAACATTTTGCTTAGTGGAAGCCAGACTGCCAGAATGCTCAAG
TACAATTATCCAACTAAATCTGGATATTGTGGGGGTGTACTATAC
AAAATTGGGCAGGTATTAGGGATACATGTGGGGGGAAATGGCAGA
GATGGCTTTTCAGCTATGTTACTCAGATCTTATTTTGCTGACACC
CAAGGCCAAATAACCTTGTCTAAAAGAACCAGTGAGTGTAATCTT
CCCAGCATACATAACCCGTGTAAAACCAAATTACAACCTAGTGTG
TTTTATGATGTGTTCCCTGGTTCAAAAGAACCAGCTGTGCTGTCT
GACAAGGATACTAGATTGCAGGTTGATTTTAATGAATCACTATTT
TCAAAATACAAGGGAAACACACATTGTGTGATGAATGAACACATA
AAAATTGCATCAGCACATTATGCAGCACAGTTGATTACTCTTGAC
ATAGATCCAAATCCTATAACACTTGAAGATAGTGTCTTTGGCACT
GATGGTCTGGAAGCTCTTGATTTGAATACTAGTGCAGGTTTTCCA
TATATTACAATGGGAATTAAAAAGAGAGACCTTATAAACAACAAG
ACCAAGGACATAAGTAAGCTTAGAGAAGCCCTTGACAAATATGGA
GTTGACTTACCAATGGTCACTTTCCTAAAAGATGAACTTAGAAAG
CAAGAGAAAATAATAAAAGGAAAAACTAGAGTTATTGAAGCTAGT
AGTGTTAATGATACTTTGCTGTTTAGAACGACCTTTGGAAACCTT
TTCTCAAAATTTCATTTGAACCCAGGTATTGTTACTGGATCAGCG
GTTGGCTGCGATCCAGAGACTTTTTGGTCGAAAATACCAGCAATG
CTAGATGATAAATGCATTATGGCCTTTGATTACACAAATTATGAC
GGTAGTATACATCCTATTTGGTTTGAAGCTCTTAAACAAGTACTC
ACAGATTTATCATTCAACCCGATGTTAATAGACAGATTATGTAGG
TCTAAACACATTTTCAAAAACACATATTATGAGGTTGAGGGAGGT
GTTCCATCTGGATGCTCGGGCACTAGTATATTTAACACTATGATA
AATAACATCATTATAAGAACTTTAGTATTAGATGCATATAAGAAC
ATAGATTTAGACAAACTTAAGATAATTGCCTATGGTGATGATGTC
ATATTTTCATACACATACGAATTGGATATGGAAGCTATAGCAATA
GAAGGTGTTAAATATGGCCTAACCATAACTCCTGCTGATAAATCT
ACTACTTTCAGAAAACTAGACTATAACAATGTTACTTTCCTAAAA
AGAGGGTTTAAGCAAGATGAAAAGTATAGTTTTTTAATCCATCCC
ACTTTTCCTGAGAATGAAATATTTGAATCAATTAGATGGACAAAG
AAACCTTCTCAAATGCACGAGCATGTTCTGTCTTTGTGTCATTTA
ATGTGGCATAATGGACGTGATGCATACAAAGGATTTGTGGAGAAG
ATACGCAGCGTTAGCGCTGGTCGCATGCTGTATATCCCTCCATAT
GATCTGCTCTTGCATGAGTGGTATGAGAAATTTTAAATAGATATA
GAAATATTAAACAATTAGTTTATTAGTTTTAGAAAAAAAAAAAAA
AAAAAAAAAAAAGACTCTCGAG
HRV-A25
(SEQ ID NO: 7)
GCGGCCGCATTTAGGTGACACTATAGATAAAACTGGATCCAGGTT
GTTCCCACCTGGATCTCCTACGTGGAGTTGTACTCTATTATTCCG
GTAATCTTGTACGCCAGTTTTATATCCCCTACCCCTTTGTAACTT
AGAAGTTAAACACACAGACCAATAGGCGGTGGCTATCCAAGTCAC
TAATGGTCAAGCACTTCTGTTTCCCCGGTCAAGATTGATACGCTC
CAACAGGGCGAAAACAGTCAAGATCGTTAACCGCAAAGTGACTAC
GCAAAGCTTAGTAATGCCTTGAAGATCTATGGCTGGTCGTTCCGC
TATAACCCCCTAGTAGACCTGGCAGATGAGGCTAGAAACACCCCA
CTGGCAACAGTGTTCTAGCCTGCGTGGCTGCCTGCACACCCTTTC
GGGTGTGAAGCCATATATTTGACAAGGTGTGAAGAGCCCCGTGTG
CTCACTTTGAGCCTCCGGCCCCTGAATGTGGCTAACCTTAACCCT
GCAGCTAGCGTATGTAATCCAACATATTGCTAGTCGTAATGAGCA
ATTGCGGGATGGGACCAACTACTTTGGGTGTCCGTGTTTCACTTT
TTTCCTTTTATTTGCTTATGGTGACAATATATATAGTATATATAT
TGGCATCCATGGGCGCACAAGTGTCTAGACAGAATGTTGGAACTC
ATTCTACTCAAAACTCAGTTTCAAATGGATCAAGTCTAAATTACT
TTAACATAAATTATTTTAAGGATGCTGCATCCAGTGGAGCTTCAA
AACTTGAATTTTCACAAGATCCTAGTAAATTCACTGACCCAGTTA
AAGATGTCCTAGAGAAAGGAATTCCAACATTACAATCTCCAACTG
TGGAAGCATGTGGCTATTCAGATAGAATAATACAAATTACAAGAG
GTGATTCCACAATAACGTCACAAGATGTGGCTAATGCAGTTATAG
GTTATGGTGTCTGGCCACATTACTTGAGTGATGAGGATGCTACTG
CTATTGACAAACCTACACAACCAGACACTTCATCTAACAGATTTT
ACACTTTGGAAAGTAAAATATGGCATACTGATTCCAAGGGATGGT
GGTGGAAGTTGCCGGATGCACTTAAAGATATGGGAATATTTGGAG
AAAACATGTACTATCACTATCTTGGTAGGAGTGGGTACACAGTAC
ATGTTCAGTGCAATGCCAGTAAATTCCATCAAGGAACGCTACTGG
TGGTGATGATACCAGAGCACCAGTTAGCAAATGCAAATTCTACTA
AAGCAGGAGCCGGGTATAATTACACACATCCTGGTGAGCGGGGAA
GACAAGTTGGTGATCAACGCGCAAACATCACTGGAGCACATCCTA
GTGATGATAACTGGCTCAATTTTGACGGCACAATGCTAGGTAATG
TCCTAATATTCCCACATCAATTTATTAATCTTAGAAGCAATAACT
CTGCAACAATAATAGTCCCTTATGTAAATGCTGTTCCAATGGATT
CAATGCTCAGACACAATAACTGGAGCCTGGTAATAATACCAATTA
GCAAGTTGCAAGCTGATAATACTGCTAGTGTTACAGTTCCAATCA
CTATATCAATAAGCCCAATGTTTGCTGAATTTTCTGGAGCACGAG
CAAGACCTACAAGAGCTGTTACAGAAGGCTTACCTACATATACAA
CACCAGGTTCAGGTCAGTTTATGACAACTGATGATTTTCAATCAC
CTTCTGCATTACCTTGGTATCATCCAACTAAAGAAATTTTAATTC
CAGGAGAAGTTAAAAATTTAATAGAAATGTGTCAGGTTGATACAC
TTATACCTATCAATAACTTAGAAAATAATTTAAGGAAAGTTAATT
TATACACTGTTGAACTAACCAATCAGACAACACCAGCCCAAAAGA
TCTTCTCTATTAAAGTGGACATTGCCTCACAACCTTTAGCAAATA
CTATGCTTGGCCAAATTGCTAGTTATTTCACACACTGGACTGGTA
GTTTACGCTTTAGTTTTATGTTTTGTGGTACAGCAAACACAACTC
TTAAATTACTTCTAGCATACACACCACCAGGCATTGGGGAACCAA
CAGACAGGAAACAAGCTATGCTTGGTACACATATAGTCTGGGATG
TTGGATTACAATCGACAGTATCTTTGGTTGTCCCGTGGGTTAGTT
CTAGTCATTTTAGATACACCTCAAGAGATGAACACTCAGCAGCAG
GATACATAACATGCTGGTACCAAACAAGTCTTGTCTTTCCTCCGA
GCACTCCTGGTACAGCTGATATGTTGTGTTTTGTATCAGCTTGTA
AGGACTTTTGTCTACGTATGGCTAGGGACACAGACATGCACACTC
AAAATGATGCCATTGAACAAAATCCAATTGAAAATTATGTGGATC
AAGTGCTTAACGAAGTTTTGGTTGTACCAAATATTAAAGAGAGCC
ACCCTAGTACATCAAACTCTGCCCCAATTTTAGATGCTGCTGAAA
CTGGACATACTAGTAATGTGCAACCAGAGGACACTATTGAAACCC
GCTATGTCCAAACCACACAAACTAGAGATGAGATGAGCATTGAGA
GTTTCCTTGGTAGATCAGGATGTGTACATACCTCAACAATTGAAA
CGACACTCAAGCACAAAGACAGATTCAAAACATGGAATATTAATC
TTAAAGAGATGGCTCAAATTAGGAGAAAGTTTGAAATGTTTACAT
ACGTAAGATTTGATTCAGAAATAACCTTAGTTCCATCTATTGCAG
GACACGGTGCAGATATAGGACACATAGTTATGCAGTACATGTATG
TACCACCTGGGGCTCCACTACCAGAAGACAGAGAACACTTCGCCT
GGCAATCAAGCACCAATGCATCAATATTTTGGCAACATGGACAAC
CCTTTCCTAGATTTTCATTACCCTTCTTGAGTGTTGCATCTGCTT
ATTATATGTTTTATGATGGTTATAATGGTGACATTCCTGGAGCAA
AATATGGTACCACTGTGGTTAATCGCATGGGGGCACTGTGTATGA
GAATTGTCACAAACAAGCAAGTTCATGATGTTGAAGTTACAACTA
ATATTTATCATAAAGCTAAACATATAAAAGCATGGTGCCCTAGAC
CACCTAGAGCTGTTCCATACAAATATGTTAACTATAATAATTATG
CAGATAGTAGTAATGTTGACATTTTTATAGAATCAAGAAGAAGTT
TACTAACAGCTGGACCAAGCGACATGTATGTCCATGTCGGTAATC
TGATATATAGAAACCTTCATCTCTTCAATTCAGAAATGCAAGATT
CAGTGTTAGTGTCATACTCTTCAGATTTAGTCATTTACCGAACAA
ACACAACAGGTAATGACTACATTCCTACTTGTGATTGTACTGATG
CCACATATTACTGTAAGCATAAAGATAGATACTATCCCATTAAGG
TCACCAGTCATGATTGGTATGAAATACAAGAGAGCGAGTACTATC
CCAAACACATCCAGTACAATCTCCTGATTGGTGAAGGACCATGTG
AGCCAGGTGACTGTGGTGGAAAACTATTGTGCAGGCATGGAGTAA
TTGGGATAGTAACTGCAGGTGGGGAGGGACATGTAGCTTTTATTG
ATCTTAGACAGTTTCATTGTGCAGATGAGCAAGGGGTGACTGATT
ATATTCATATGTTAGGTGAGGCTTTTGGTGCAGGTTTTGTTGATA
GTGTTAAGGAGCAAATAAATGCCATTAATCCAATTAATAATATTA
GCAAGAAAATAATTAAATGGCTTCTTAGAATAATTTCGGCAATGG
TCATAATTATTAGAAATTCCTCTGATCCACAAACAATTATAGCTA
CATTGACTTTGATTGGGTGTTCAGGTTCCCCTTGGAGATTCCTCA
AGGAAAAATTTTGTAAATGGACACAGTTGAATTACATACATAAAG
AATCTGATTCATGGCTCAAAAAATTTACTGAAATGTGTAATGCAG
CCAGAGGTTTGGAGTGGATTGGGAATAAAATATCAAAATTTATTG
AATGGATGAAATCCATGCTTCCTCAAGCTCAACTTAAAGTTAAAT
ATCTGAGTGAACTTAAAAAATTAAACTTTCTTGAAAAGCAGATAG
AGCACCTTAGAGTTGCTGATTCCCAAACTCAGGAAAAAATTAAGC
ATGAGATTGACACTTTGCATGACCTTTCTTGTAAATTTCTTCCAC
TTTATGCTAGTGAAGCAAAAAGAATTAAGATATTACACAATAAGT
GTAGCACAATTATCAAACAAAAGAAAAGGTGTGAGCCAGTTGCTG
TTGTTATACATGGAGAGCCTGGTACTGGTAAGTCTATGACCACAA
ATTTCCTTGCCAGAATGATAACAAGTGATAGTGACATATACTCTT
TACCACCTGACCCCAAATATTTTGATGGATATGACCAACAAAGTG
TGGTCATTATGGATGACATCATGCAAAATCCAAGTGGTGAAGACA
TGACCTTATTTTGTCAGATGGTGTCTAGTGTTACATTCATACCCC
CAATGGCTGATTTGCCTGATAAAGGTAAACCTTTTGATTCAAGAT
TTGTCTTGTGTAGTACTAATCATAATATGCTAGCACCACCTACAA
TCACTTCATTGCCTGCTATGAATAGGAGATTCTATTTTGACCTAG
ATATTGTAGTATGTGACAAATATAAAGATTCACAAGGAAAGCTTA
ATGTGTCAGCTGCTTTTCAACCATGCGATGTTGACACAAAAATTG
GAAATGCCCGTTGCTGTCCTTTTATTTGTGGTAAAGCTGTCATCT
TTAGGGACCGCAATACCTGTCAAACCTACACCTTGGCACAAGTGT
ATAACCAAATGCTTGCTGAAGACAAAAGGCGCAGGCAAGTTATTG
ATGTAATGGCAGCAATTTTCCAAGGCCCTATTACTATTGAAGGAC
CACCACCTGCAGCAATAAACGACTTACTGAAATCAGTTAGAACTC
CAGAAGTAATTAGATATTGTGAAGAGAATAAATGGATAGTGCCTG
CTGAGTGCAAAGTTGAAAGAGACCTTAATATAGCAAATACAATTA
TTACAATTATAGCTAATATTATTAGTATATCTGGAATAATTTATG
TAATTTACAAACTTTTCTGCACATTGCAAGGACCATACTCAGGTG
AGCCTAAGCCAAAGACCAAGATACCAGAGAGGAGAGTTGTGGCTC
AGGGTCCAGAAGAAGAATTTGGTAGATCACTCATTAAACATAATT
CATGTATAGTGTCAACGCAGAATGGAAAATTCACAGGCTTAGGGA
TTTATGATAGAACATTAATCTTACCAACACATGCAGATCCTGGTA
ATGAGATACAGATTGATGGAATTACAACTAAAGTAGAGGATTCTT
ATGATTTGTATAATAAAGATGGAATAAAGCTAGAAATTACAGTCC
TAAAACTTAAGAGAAATGAAAAGTTCAAAGACATTAGGAAATACA
TACCAGAGAATGAAGATGATTATCCAGATTGTAATTTGGCCTTAT
CTGCCAATCAACCAGAAACAACCATAATCAATGTAGGGGATGTAG
TATCTTATGGCAATATTTTACTTAGTGGAAATCAGACAGCTAGGA
TGCTTAAATATAATTACCCAACAAAATCAGGTTATTGTGGTGGCA
TCCTGTATAAAATTGGTCAAGTCCTTGGAATACATGTTGGAGGAA
ATGGGAGAGATGGATTTGCTGCCATGCTGCTACGATCTTACTTTT
CTGAGACACAAGGTCAAATAATTACTTCCAAATCTACAAGTGAGT
GTGGTTTACCTAGTATCCACACACCAAACAAAACAAAGTTACAAC
CTAGTGTTTTTTATGATGTGTTTCCTGGTAATAAAGAGCCAGCTG
TTCTTACAAGTGATGACCCTAGGCTAGAAGTAGATTTCAAAGAAG
CTTTGTTTTCTAAATATAAAGGAAATAATCAGTGTACCATGAATG
ATCATATTAAAGTGGCTATTTCTCATTACTCAGCACAATTGATGA
CATTAGATATTGATCCAAAACCTATATCCTTAGAAGAAAGTGTTT
TTGGCATAGAGGGCTTGGAAGCACTAGATCTCAACACCAGTGCAG
GATTTCCTTATGTATCTATGGGCATTAAGAAAAGAGATTTAATTA
ATAAACAAACTAAAGATCTGACAAAATTAAAGATGGCATTAGATA
AGTACGGTGTTGATTTGCCTATGGTAACCTTTCTTAAAGATGAGC
TTAGGAAAAAGGAGAAAATCTGTGCTGGAAAGACCCGTGTAATTG
AAGCTAGTAGTGTAAATGATACTGTGTTATTTAGAACAACTTTTG
GCAATCTTTTTTCAAAATTTCACTTAAATCCAGGTATTATCACTG
GCTCTGCTGTGGGATGTGATCCAGAAACATTTTGGTCAAAAATAC
CAGTTATGCTAGATGGAGAATGTATAATGGCCTTTGATTATACAA
ATTATGATGGGAGTATACATCCTATTTGGTTTCAAGCCTTGAAAG
AAGTCTTAGTCAATTTATCATTTGAATCAACTTTAATAGATAGGT
TGTGCAGATCCAAACATATCTTCAAAAACACATATTATGAGGTTG
AGGGCGGGGTTCCATCAGGTTGCTCTGGTACTAGCATATTTAACA
CCATGATCAACAACATAATCATAAGGACTTTGGTGCTGGATGCTT
ACAAAAATATAGATTTAGATAAGTTAAAGATCTTGGCTTATGGGG
ATGATGTAATCTTTTCCTACAAATATCAGCTTGACATGGAAGCAA
TTGCTAAAGAAGGCGTCAAGTATGGGCTGACAATAACACCTGCTG
ACAAATCCAATGATTTTAAGCAGCTTAATTATGACAATGTAACAT
TTCTCAAAAGAGGATTTAGACAGGATGAAAAGCATCAATTTCTGA
TACACCCAACATTTCCAATTGAAGAGATCCAAGAATCCATACGAT
GGACAAAGAAACCATCTCAAATGCAGGAACATGTACTGTCATTGT
GTCACTTAATGTGGCATAATGGCAGAGATGTGTACAAGAAGTTTG
AGGAGCGTATACGTAGTGTTAGCGCTGGTCGTGCACTATATATTC
CTCCTTACGACTTACTACTGCATGAGTGGTATGAAAAATTTTAAA
TTGTTTAGATATAGAAATATTAAACATTTAGTTAAAAAAAAAAAA
AAAAAAAAAAAAAGACTCTCGAG
HRV-A45
(SEQ ID NO: 8)
TAATACGACTCACTATAGTTAAAACTGGGTCGTGGTTGTTCCCAC
CACGACTATCTACGGATGTAGTGCTCTTGTATTCCGGTACGCTTG
CACGCCAGTTTTGTCACCCCCCCCTACATTGTAACTTAGAAGATT
AACACAAAGACCAATAGGCGGCAATGAACCACATTGTCAACGGTC
AAGCACTTCTGTTTCCCCGGTCAATCTAGATATGCTTTACCAAAG
GCAAAAACTGGAGCGATCGTTATCCGCAAGGTGCCTACGGGAAAC
CCAGTAGCATTCTTTATTTGATCTGGTTGGTTGCTCAGCCGTTAA
ACCCAAACGGTAAACCTGGCAGATGAGGCTGGAAGATCCCCACCA
GCGATGGTGTTCCAGCCTGCGTGGCTGCCTGCACACCCGAAAGGG
TGTGAAGCCTTTTCAAAGACAGGGTGCGAAGAGTCTACTGTGCTC
ACCTTGATTCCTCCGGCCCCTGAATGTGGCTAATCCTAACCCCGT
AGCTGTGGTGTGCAATCCAGCACATTCGCAGTCGTAATGGGTAAC
TGCGGGATGGAACCAACTACTTTGGGTGTCCGTGTTTCTCTTTTT
CCTTTTATATATGCTTATGGTGACAATTATACGGATATAGTTGTC
ATCATGGGCGCTCAAGTCTCAAGGCAGAATGTTGGTACCCACTCT
ACACAAAACACAGTTACAGGAGGATCCAGTCTTAATTATTTTAAT
ATTAATTACTTCAAAGATGCTGCTTCATCTGGAGCGTCAAGGTTG
GACTTTTCTCAAGACCCAAGTAAATTTACAGACCCAGTCAAAGAT
GTTTTAACCAAGGGTATCCCAACCCTACAGTCACCTACAGTAGAA
GCTTGTGGGTATTCAGATAGAATAATACAAATTACCAGAGGAGAC
ACAACAATAACATCCCAGGACATTGCAAACGCTGTAGTTGGATAT
GGAGTTTGGCCTACTTATTTAGATTCAAAGGATGCCTCAGCTATA
GATAAGCCCACACAACCAGATACTTCTGCAAATAGGTTTTACACA
CTGGAAAGTAAGGAATGGACTCCAGATAGTAAAGGATGGTGGTGG
AAATTACCAGATGCACTCAAAGATATGGGTGTTTTTGGTGAAAAC
ATGTTCTACCATGCACTTGGTAGGTCTGGTTACCTAATTCATGTT
CAGTGCAATGCAAGTAAATTTCATTCAGGGACCCTTCTTGTTGTT
GCTATCCCAGAGCACCAGTTGGCATATATAGGCACAGGAAATGTA
ACAGTAGGATACAAACACACCCATCCTGGAGAGACTGGAAGAGTC
ATAGCAACAACCACAGACAAACAGACAAGACAGCCATCGTTTGAC
AGTTGGCTCAATTGTAATGGTACCCTTTTAGGCAATGCCTTAATA
TTTCCACATCAATTTATCAATTTGAGAACAAACAATGCTGCCACC
TTGATCCTGCCGTACGTAAATGCAACTCCCATGGATTCCATGTTA
AGACATAATAATTGGTCATTACTAATAGTGCCTGTATCAGAACTA
AGAGGTGACACCAGTATACCAATTACTGTATCAATCTCTCCCATG
GCAGCTGAATTTTCTGGAGCACGGAACCGCAGTGCACGTGTGGAA
GGTTTACCAGTGATGTTAACACCAGGATCAGGACAATTCTTGACA
ACTGATGACATGCAATCACCATCAGTTTTACCTTACTTCCACTCA
ACACAAGAAATCTTCATACCAGGAGAAGTTAAAAATCTTATTGAA
TTGTGCCAAGTGGACACCATGGTACCATTAAACAACTTGCACATA
AATAAAAACAAGATTGGAATGTATGCTCTACCACTTACACGACAG
AATACACCAGCTGCTGAACTCTTTGCAATGCCAGTCGACATTACA
TCTTCACCTTTAGCAACCACGCTTTTAGGAGAGATTGCTTCTTAT
TACACCAACTGGACAGGTAGTATAAGATTGAGTTTCATGTTTTGT
GGCAGTGCAAACACCTCACTCAAGCTTCTTCTAGCATATACTCCA
CCTGGAGTGAGTAAACCAACCAGTAGGAGAGAAGCTATGCTGGGC
ACCCATCTTGTATGGGATGTAGGTCTTCAATCCACATGTTCTTTA
GTCATCCCGTGGATATCAGCATCACATTTTCGGAATACCACGCCA
GACACCTACTCAAAAGCTGGTTATGTGACATGCTGGTATCAGACA
AATTTCATCACAGCACCAAACACACCACCTACAGCTGACATTATT
TGTCTGGTTTCAGCATGTAAGGATTTTTGCTTACGCATGGCTAGA
GATACTAACCAGCACACTCAGCTTGGAGCAATAGAGCAAAACCCT
GTTGAACAATTTGCAGAAGCAGTCCTTGATCAAGTATTAGTAGTT
CCAAACACTCGACCCAGCGATGGGTTGATTGCAAACTCAGCCCCA
GCTTTGGATGCAGCTGAAACTGGACACACCAGTTCAGTGCAGCCT
GAGGACCTTATAGAGACTAGATATGTGATTGCAGACCAAACCAGA
CATGAAACCTCCATTGAATCTTTTTTGGGTAGGGCTGGATGTGTG
GCCACTATTAGTTTAGACATTAACCATGATGACTACCAAAAGAAT
TACAAAAATTGGGCAATTAGTTTACAAGAAATGTCACAAATTAGG
AGGAAATTTGAAATGTTTACATATGTCAGATTTGATTCCGAAATA
ACAATAGTACCATGTGTTGCTGCCACAGAAGGTAACTTGGGACAC
ATTGTTGTGCAATACATGTTTGTACCACCAGGAGCACCTCTCCCT
GTTAGTAGAACTGACAACACTTGGCAATCTAGCACAAATGCATCA
GTCTTTTGGCAGGTTGGTCAAACTTATCCCAGATTTTCTATACCT
TTCTCAAGTATAGCTTCAGCTTACTACATGTTTTATGATGGATAC
GACACTGATGGCACAGATGCAGTGTATGGTGTTAGTGTGACTAAC
GATATGGGGACTATATGTGTTAGAATTGTTACAGACCAACAACAA
CATAGAGTTAAGATCGACTCCATGGTATATCTAAAAGCTAAACAC
ATCAAGGCATGGTGTCCCAGACCTCCAAGAGCAGTCACATATAAC
CATACATATAATCCAAATTATGTTAGGGCTGATGAAACAGCCACA
AAAGTCCAAACTAGAGCAAATGTCACAACAGTAGGTCCATCAGAC
ATGTTTATTCACGCCTCAGAGTTTTTATACAGAAATTACCATCTC
ACTCCAGAAAAGGAACTGAAAGAAGCATGCCAAATTGTATATACT
GCAGATCTAGTTATACACCGCACAAGAGACAAAGGTGATGACTAT
ATCCCACAATGCAATTGTACAGATTGCTGTTACTACTGTGCCCAC
AAAGACAGGTATATTCCCATCAAAGTAGAATACCATAGCTACTAC
ACCATCCAGAAATCAGATTATTATCCAAAACATATACAGTATGAT
ATACTAATTGGTGAGGGACCTTCGCAACCAGGTGATTGTGGAGGA
AAACTTTTATGCAGACATGGTGTTATTGGTATGGTGACAGCTGGA
GGGGAAGGACATGTAGCATTTACTGATTTGAGGAAGTATAGAATG
GTAGAGGCTGAGGAGCAAGGTATAACAGATTATGTTAAATCCTTA
GGTGATGCTTTTGGAGTAGGATTTGTAGATCAAATTAAAGAACAA
ATTAATAATATAAACCCATTAAATAAAATCAGTGCTAAAGTGATC
AAATGGCTAATCAGAGTAATATCAGCACTAGTGATAGCAGTGCGT
AGCCAAGGGGATCCAGCAACACTATCAGCTACTCTACTTTTACTT
GGGTGCTCTAATTCCCCGTGGCGGTTCCTGAAACAGAAGGTGTGT
ACATGGTTGGGGCTTAGATACATACACAAAGAATCAGATGGTTGG
ATCAAGAAATTTACTGAAATGTGCAATGCAGCCAGAGGCTTAGAA
TGGATAGGGTGTAAGATTTCAAAATTCATTGACTGGCTGAAATCC
ATGCTACCTCAAGCACAGAACAAAATCAAGTTTCTCCACTTCATG
AAACAATTGCAGCTAAAAGAAAAACAGATCGATGGTTTACCTTAT
GCAACAGTTAAGCAACAAGAAGATTATCTCAAAGAAATGGAAGAG
ATGTTGGACATTTCAAATAAACTATTACCACTATACCCAAAGGAG
AATAAGATTATAAAAGATCTACTCAAACAAGCTAAAAGCATGACA
ACAACATCAAAGAGAGTTGAACCAGTTGCAATCATGTTTCATGGA
GATCCAGGGTCAGGGAAATCAGTGTGCACAAACATCCTTGCCCGC
ATGATAACTAATCCATCAGACATATATTCCCTACCCCCAAATCCA
AAGTATTTTGATGGATACCATCAACAGACTGTAGTAATAATGGAT
GATGTGATGCAAAACCCAGATGGGGAAGACATGAGCACCTTCTGC
CAAATGGTTTCCTCAGTTAATTATGTAGTGCCAATGGCTGATCTA
CCAGATAAGGGTACCCTGTTTTCATCAGATTATGTCTTTTGCAGT
ACAAATCAGCATGTTTTGATTCCTCCAACCATATCTACCATACCC
GCCCTGAATCGGCGATTGTTCTTCGATCTAACTGTTAAGGTGAAC
CCCAGATATCAGGAAGCAGGAAAACTAAATCTGGACTGTGCATTA
AAACCATGCAATCATGAGTTAAAGGTTGGAAATGCAAGGTGTTGT
CCTCTCATTTGTGGTAAGGCTATAACCTTCATTAACAGACACAAC
AATGAAGAATTGACACTGTCCCAAATATACAATCAAGTAGTAAAT
GAACACAACCGCAGATTAAACGTGTCAAAACACATGGAAGCAATT
TTCCAGGGACCTATTGATATGCAAGCACCTCCCCCACCAGCAATA
GTTGATTTACTTAGATCCACCAGAAATGAAGATGTCATCAACTAC
TGCAAAAACCAAAATTGGATTATTCCTGCTGACATCAGTATTGAG
AGAGAATTGAATCTGGTAAATCTTTCAATATCAATTCTGGCAAAT
CTAATTAGTGTGATAGGAATCATTTACATTATATATAAATTATTT
GTATCATTACAAGGACCTTATAGTGGACTACCCACCAAGAAAAAA
GTGATTCCAGAGAAAAGAGTGGTAGTACAAGGTCCTTCTACTGAA
TTTGGCCTAAGTTTAATAAAACATAACACATGTATTGTAGAAACA
GAAAATGGTAAGTTTACAGGTTTAGGAATATACGATAACCTATTA
GTCATCCCCACACATGCTGATCCCAGTAGAACTGTCAAGGTGGAT
GGAGTGGAAACAGAAGTGTTGGACTCTTACGATTTGTACAACAAA
GAAGGAGTAAAATTAGAAATCACAGTTCTGATCTTATCCAGAAAT
GAAAAGTTTAGGGATATTAGAAAATACATACCAAATTCTGAGGAT
GATTATGTCAACTGTAATCTGGCTTTAGTTGCAAACCAAGATATG
CCACAGATACTGGAGGTCGGAGATGTTGTCTCTTATGGCAATATA
CTCTTGAGTGGTAATAACACCGCCAGAATGCTCAAATATGAATAC
CCTACAAAATCGGGTTTTTGTGGAGGTGTTTTGTACAAGGTGGGT
CAAGTAATTGGCATACACGTAGGTGGCAATGGAAGACAAGGATTC
TCAGCCATGTTACTTAAAAGATATTTCAACCAACAACAAGGTCAG
ATAATTCTAAAGAAACCAGTCAAGGAAGTTGATTATCCCAGTATA
CATACCCCTACTAAGACAAAACTCCAACCAAGTGTTTTCCATGAT
ATTTTTCCCGGAGTTAAGGAACCTGCAGTTCTAACAGAAAAAGAC
CCCCGGTTGGAAGTAGACTTGAACTCCTCTCTTTTTTCAAAATAT
GCTGGAAATGTTAATTTAGAAATGAATGAGTACATGATTGTTGCA
GCATCCCATTATGCATCACAACTTGAAACACTAGACATATCCAAC
CAGCAAATGTCTATTGAGGAATGTGTGTATGGAACAGATAACTTA
GAAGCCTTGGATTTAAATACTAGTGCAGGATTTCCCTATGTGGCT
TTGGGAATTAAGAAGAAGGACTTAATAAATAGAGAGACTAGAGAT
GTCAGTAAAATTAAAAATTGCTTAGACACTTATGGTGTAGATTTA
CCAATGATAACATATTTAAAAGATGAGCTCAGAACACCAGAAAAA
ATAAAGTTAGGAAAAACTAGGGTAATAGAAGCAAGTAGTTTAAAT
GATACTATTCATATGAGAATGCTCTTTGGAAACTTGTTTAAAGCC
TTTCACGCAAATCCAGGCATAGTAACAGGGTGTGCAGTTGGTTGT
GATCCAGAAACATTTTGGTCTAAAATTCCTCCAATGCTGGGTGAT
GGGTGTGTGATGGCATTTGATTATACAAATTATGATGGAAGTTTA
CATCCAATATGGTTTAGGCTACTTGAAAGAGTGCTGGATAGGTTG
GGATTCCCAGGCTGTGCAGTTAGAAAATTATCACACTCCACCCAT
ATATACAAAGGAATGTACTATGAAGTTGATGGAGGAATGCCATCT
GGGTGTGCAGGTACTTCAATCTTCAACTCAATGATCAATAACATC
ATAATCAGAACAATAATTCTACATGCTTACAAGAATATAGACCTG
GATCAGTTGAGGATACTTACATATGGTGATGATATAATTTTTACT
TACCCAGATAAACTAGACATGGCTTATTTAGCCCAAATTGGAGAA
AAATATGGTTTAAAAATGACACCTGCAGACAAGTCAGATACATTT
AAGGATTTGGATCTGAGTACAGCAACTTTCCTAAAAAGAGGCTTT
AAACCTGACTCAAAACACCCATTTTTAGTCCATCCCATATATCCA
ATCCAGGATATCTATGAATCAATCAGATGGACAAAGAATCCCAGA
TGTTTACAGGAGCATGTGCTCAGTTTGGCTCACTTATGCTGGCAT
AGTGGACCAGAGCAGTATGCTGATTTTGTCAAGAGAATCAGATCA
ACATCTGTTGGGAAGAACCTATACATACCATCTTATGATGTACTA
CTTTATGAGTGGTATGAGAAATTTTAAGTTAATATATACAGTTAC
TATTTAGGTAGTTTGGGTGTATAAAAAAAAAAAAAAAAAAAAAAA
AAAAAAAAAAAAAAAAAAAAAAAAAAAGAAGAGCTTTAGTGAGGG
TTAATT
HRV-B14
(SEQ ID NO: 9)
TAATACGACTCACTATAGTTAAAACAGCGGATGGGTATCCCACCA
TTCGACCCATTGGGTGTAGTACTCTGGTACTATGTACCTTTGTAC
GCCTGTTTCTCCCCAACCACCCTTCCTTAAAATTCCCACCCATGA
AACGTTAGAAGCTTGACATTAAAGTACAATAGGTGGCGCCATATC
CAATGGTGTCTATGTACAAGCACTTCTGTTTCCCAGGAGCGAGGT
ATAGGCTGTACCCACTGCCAAAAGCCTTTAACCGTTATCCGCCAA
CCAACTACGTAACAGTTAGTACCATCTTGTTCTTGACTGGACGTT
CGATCAGGTGGATTTTCCCTCCACTAGTTTGGTCGATGAGGCTAG
GAATTCCCCACGGGTGACCGTGTCCTAGCCTGCGTGGCGGCCAAC
CCAGCTTATGCTGGGACGCCCTTTTAAGGACATGGTGTGAAGACT
CGCATGTGCTTGGTTGTGAGTCCTCCGGCCCCTGAATGCGGCTAA
CCTTAACCCTAGAGCCTTATGCCACGATCCAGTGGTTGTAAGGTC
GTAATGAGCAATTCCGGGACGGGACCGACTACTTTGGGTGTCCGT
GTTTCTCATTTTTCTTCATATTGTCTTATGGTCACAGCATATATA
TACATATACTGTGATCATGGGCGCTCAGGTTTCTACACAGAAAAG
TGGATCTCACGAAAATCAAAACATTTTGACCAATGGATCAAATCA
GACTTTCACAGTTATAAATTACTATAAGGATGCAGCAAGTACATC
ATCAGCTGGTCAATCACTGTCAATGGACCCATCTAAGTTTACAGA
ACCAGTTAAAGATCTCATGCTTAAGGGTGCACCAGCATTGAATTC
ACCCAATGTTGAGGCCTGTGGTTATAGTGATAGAGTACAACAAAT
CACACTCGGGAATTCAACAATAACAACACAAGAAGCAGCCAACGC
TGTTGTGTGTTATGCTGAATGGCCAGAGTACCTTCCAGATGTGGA
CGCTAGTGATGTCAATAAAACTTCAAAACCAGACACTTCTGTCTG
TAGGTTTTACACATTGGATAGTAAGACATGGACAACAGGTTCTAA
AGGCTGGTGCTGGAAATTACCAGATGCACTCAAGGATATGGGTGT
GTTCGGGCAAAACATGTTTTTCCACTCACTAGGAAGATCAGGTTA
CACAGTACACGTTCAGTGCAATGCCACAAAATTCCATAGCGGTTG
TCTACTTGTAGTTGTAATACCAGAACACCAACTGGCTTCACATGA
GGGTGGCAATGTTTCAGTTAAATACACATTCACGCATCCAGGTGA
ACGTGGTATAGATTTATCATCTGCAAATGAAGTGGGAGGGCCTGT
CAAGGATGTCATATACAATATGAATGGTACTTTATTAGGAAATCT
GCTCATTTTCCCTCACCAGTTCATTAATCTAAGAACCAATAATAC
AGCCACAATAGTGATACCATACATAAACTCAGTACCCATTGATTC
AATGACACGTCACAACAATGTCTCACTGATGGTCATCCCTATTGC
CCCTCTTACAGTACCAACTGGAGCAACTCCCTCACTCCCTATAAC
AGTCACAATAGCACCTATGTGCACTGAGTTCTCTGGGATAAGGTC
CAAGTCAATTGTGCCACAAGGTTTGCCAACTACAACTTTGCCGGG
GTCAGGACAATTCTTGACCACAGATGACAGGCAATCCCCCAGTGC
ACTGCCAAATTATGAGCCAACTCCAAGAATACACATACTAGGGAA
AGTTCATAACTTGCTAGAAATTATACAGGTAGATACACTCATTCC
TATGAACAACACGCATACAAAAGATGAGGTTAACAGTTACCTCAT
ACCACTAAATGCAAACAGGCAAAATGAGCAGGTTTTTGGGACAAA
CCTGTTTATTGGTGATGGGGTCTTCAAAACTACTCTTCTGGGTGA
AATTGTTCAGTACTATACACATTGGTCTGGATCACTTAGATTCTC
TTCGATGTATACTGGTCCTGCCTTGTCCAGTGCTAAACTCACTCT
AGCATACACCCCGCCTGGTGCTCGTGGTCCACAGGACAGGAGAGA
AGCAATGCTAGGTACTCATGTTGTCTGGGATATTGGTCTGCAATC
CACCATAGTAATGACAATACCATGGACATCAGGGGTGCAGTTTAG
ATATACTGATCCAGATACATACACCAGTGCTGGCTTTCTATCATG
TTGGTATCAAACTTCTCTTATACTTCCCCCAGAAACGACCGGCCA
GGTCTACTTATTATCATTCATAAGTGCATGTCCAGATTTTAAGCT
TAGGCTGATGAAAGATACTCAAACTATCTCACAGACTGTTGCACT
CACTGAAGGCTTAGGTGATGAATTAGAAGAAGTCATCGTTGAGAA
AACGAAACAGACGGTGGCCTCAATCTCATCTGGTCCAAAACACAC
ACAAAAAGTCCCCATACTAACTGCAAACGAAACAGGGGCCACAAT
GCCTGTTCTTCCATCAGACAGCATAGAAACCAGAACTACCTACAT
GCACTTTAATGGTTCAGAAACTGATGTAGAATGCTTTTTGGGTCG
TGCAGCTTGTGTGCATGTAACTGAAATACAAAACAAAGATGCTAC
TGGAATAGATAATCACAGAGAAGCAAAATTGTTCAATGATTGGAA
AATCAACCTGTCCAGCCTTGTCCAACTTAGAAAGAAACTGGAACT
CTTCACTTATGTTAGGTTTGATTCTGAGTATACCATACTGGCCAC
TGCATCTCAACCTGATTCAGCAAACTATTCAAGCAATTTGGTGGT
CCAAGCCATGTATGTTCCACATGGTGCCCCGAAATCCAAAAGAGT
GGGCGATTACACATGGCAAAGTGCTTCAAACCCCAGTGTATTCTT
CAAGGTGGGGGATACATCAAGGTTTAGTGTGCCTTATGTAGGATT
GGCATCAGCATATAATTGTTTTTATGATGGTTACTCACATGATGA
TGCAGAAACTCAGTATGGCATAACTGTTCTAAACCATATGGGTAG
TATGGCATTCAGAATAGTAAATGAACATGATGAACACAAAACTCT
TGTCAAGATCAGAGTTTATCACAGGGCAAAGCTCGTTGAAGCATG
GATTCCAAGAGCACCCAGAGCACTACCCTACACATCAATAGGGCG
CACAAATTATCCTAAGAATACAGAACCAGTAATTAAGAAGAGGAA
AGGTGACATTAAATCCTATGGTTTAGGACCTAGGTACGGTGGGAT
TTATACATCAAATGTTAAAATAATGAATTACCACTTGATGACACC
AGAAGACCACCATAATCTGATAGCACCCTATCCAAATAGAGATTT
AGCAATAGTCTCAACAGGAGGACATGGTGCAGAAACAATACCACA
CTGTAACCGTACATCAGGTGTTTACTATTCCACATATTACAGAAA
GTATTACCCCATAATTTGCGAAAAGCCCACCAACATCTGGATTGA
AGGAAGCCCTTATTACCCAAGTAGATTTCAAGCAGGAGTGATGAA
AGGGGTTGGGCCGGCAGAGCTAGGAGACTGCGGTGGGATTTTGAG
ATGCATACATGGTCCCATTGGATTGTTAACAGCTGAAGGTAGTGG
ATATGTTTGTTTTGCTGACATACGACAGTTGGAGTGTATCGCAGA
GGAACAGGGGCTGAGTGATTACATCACAGGTTTGGGTAGAGCTTT
TGGTGTCGGGTTCACTGACCAAATCTCAACAAAAGTCACAGAACT
ACAAGAAGTGGCGAAAGATTTCCTCACCACAAAAGTTTTGTCCAA
AGTGGTCAAAATGGTTTCAGCTTTAGTGATCATTTGCAGAAATCA
TGATGACTTGGTCACTGTTACGGCCACTCTAGCACTACTTGGATG
TGATGGATCTCCTTGGAGATTTCTGAAGATGTACATTTCCAAACA
CTTTCAGGTGCCTTACATTGAAAGACAAGCAAATGATGGATGGTT
CAGAAAGTTTAATGATGCATGTAATGCTGCAAAGGGATTGGAATG
GATTGCTAATAAGATTTCCAAACTGATTGAATGGATAAAAAACAA
AGTACTTCCCCAAGCCAAAGAAAAACTAGAATTTTGTAGTAAACT
CAAACAACTTGATATACTAGAGAGACAAATAACCACCATGCATAT
CTCGAATCCAACACAGGAAAAACGAGAGCAGTTGTTCAATAACGT
ATTGTGGTTGGAACAAATGTCGCAAAAGTTTGCCCCATTTTATGC
CGTTGAATCAAAAAGAATCAGGGAACTCAAGAACAAAATGGTAAA
TTATATGCAATTTAAAAGTAAACAAAGAACTGAACCAGTGTGTGT
ATTAATCCATGGTACACCCGGTTCTGGTAAATCATTAACAACATC
CATTGTGGGACGTGCAATTGCAGAACACTTCAATTCAGCAGTATA
TTCACTTCCACCAGATCCCAAGCACTTTGATGGTTATCAGCAACA
GGAAGTTGTGATTATGGATGATCTGAACCAAAATCCAGATGGACA
GGATATAAGCATGTTTTGTCAAATGGTTTCTTCAGTGGATTTCTT
GCCTCCAATGGCTAGTTTAGATAACAAGGGCATGTTATTCACCAG
TAATTTTGTTCTAGCCTCCACAAATTCTAACACACTAAGCCCCCC
AACAATCTTGAATCCTGAAGCTTTAGTCAGGAGATTTGGTTTTGA
CCTAGATATATGTTTGCATACTACCTACACAAAGAATGGAAAACT
CAATGCAGGCATGTCAACCAAGACATGCAAAGATTGCCATCAACC
ATCTAATTTCAAGAAATGTTGCCCCCTAGTCTGTGGAAAAGCTAT
TAGCTTGGTAGACAGAACTACCAACGTTAGGTATAGTGTGGATCA
ACTGGTCACGGCTATTATAAGTGATTTCAAGAGCAAAATGCAAAT
TACAGATTCCCTAGAAACACTGTTTCAAGGACCAGTGTATAAAGA
TTTAGAGATTGATGTTTGCAACACACCACCTTCAGAATGTATCAA
CGATTTACTGAAATCTGTAGATTCAGAAGAGATTAGGGAATATTG
TAAGAAGAAGAAATGGATTATACCTGAAATTCCTACCAACATAGA
AAGGGCTATGAATCAAGCCAGCATGATTATTAATACTATTCTGAT
GTTTGTCAGTACATTAGGTATTGTTTATGTCATTTATAAATTGTT
TGCTCAAACTCAAGGACCATATTCTGGTAACCCGCCTCACAATAA
ACTAAAAGCCCCAACTTTACGCCCAGTTGTTGTGCAAGGACCAAA
CACAGAATTTGCACTATCCCTGTTAAGGAAAAACATAATGACTAT
AACAACCTCAAAGGGAGAGTTCACAGGGTTAGGCATACATGATCG
TGTCTGTGTGATACCCACACACGCACAGCCTGGTGATGATGTACT
AGTGAATGGTCAGAAAATTAGAGTTAAGGATAAGTACAAATTAGT
AGATCCAGAGAACATTAATCTAGAGCTTACAGTGTTGACTTTAGA
TAGAAATGAAAAATTCAGAGATATCAGGGGATTTATATCAGAAGA
TCTAGAAGGTGTGGATGCCACTTTGGTAGTACATTCAAATAACTT
TACCAACACTATCTTAGAAGTTGGCCCTGTAACAATGGCAGGACT
TATTAATTTGAGTAGCACCCCCACTAACAGAATGATTCGTTATGA
TTATGCAACAAAAACTGGGCAGTGTGGAGGTGTGCTGTGTGCTAC
TGGTAAGATCTTTGGTATTCATGTTGGCGGTAATGGAAGACAAGG
ATTTTCAGCTCAACTTAAAAAACAATATTTTGTAGAGAAACAAGG
CCAAGTAATAGCTAGACATAAGGTTAGGGAGTTTAACATAAATCC
AGTCAACACGGCAACTAAGTCAAAATTACATCCCAGTGTATTTTA
TGATGTTTTTCCAGGTGACAAGGAACCTGCTGTATTGAGTGACAA
TGATCCCAGACTGGAAGTTAAATTGACTGAATCATTATTCTCTAA
GTACAAGGGGAATGTAAATACGGAACCCACTGAAAATATGCTTGT
GGCTGTAGACCATTATGCAGGGCAACTATTATCACTAGATATCCC
CACTTCTGAACTTACACTAAAAGAAGCATTATATGGAGTAGATGG
ACTAGAACCTATAGATATTACAACCAGTGCAGGATTTCCCTATGT
GAGTCTTGGGATCAAAAAGAGAGACATTCTGAATAAAGAGACCCA
GGACACAGAAAAGATGAAGTTTTATCTAGACAAGTATGGCATTGA
CTTGCCTCTAGTTACATATATTAAGGATGAATTAAGAAGTGTTGA
CAAAGTCCGATTAGGGAAAAGTAGATTAATTGAAGCCTCCAGTTT
GAATGATTCTGTTAACATGAGAATGAAACTAGGCAACCTTTACAA
AGCATTCCATCAAAATCCCGGTGTTCTGACTGGATCAGCAGTGGG
TTGTGATCCTGATGTGTTTTGGTCTGTCATCCCTTGCTTAATGGA
TGGGCACCTGATGGCATTTGATTACTCTAATTTTGATGCCTCTTT
GTCACCAGTTTGGTTTGTCTGTCTAGAGAAGGTTTTGACCAAGTT
AGGCTTTGCAGGCTGTGCATTAATTCAATCAATTTGTAATACCCA
TCATATCTTTAGGGATGAAATATATGTGGTTGAAGGTGGCATGCC
CTCAGGGTGTTCAGGAACCAGCATATTCAATTCCATGATCAACAA
CATAATCATTAGGACTTTGATATTAGATGCATATAAAGGAATAGA
TTTAGACAAACTTAAAATCTTAGCTTACGGTGATGATTTGATTGT
TTCTTATCCTTATGAACTGGATCCACAAGTGTTGGCAACTCTTGG
TAAAAATTATGGACTAACCATCACACCCCCAGACAAATCTGAAAC
TTTTACAAAAATGACATGGGAAAACTTGACATTTTTAAAGAGATA
CTTCAAGCCTGATCAACAATTTCCCTTTTTGGTTCACCCAGTTAT
GCCCATGAAAGATATACATGAGTCAATCAGATGGACAAAGGATCC
TAAAAACACACAGGATCACGTCCGATCATTATGCATGTTAGCATG
GCACTCAGGAGAAAAAGAGTACAATGAATTCATTCAGAAGATCAG
AACTACTGACATTGGAAAATGTCTAATTCTCCCAGAATACAGCGT
ACTTAGGAGGCGCTGGTTGGACCTCTTTTAGGTTAACAATATAGA
CACTTAATTTGAGTAGAAGTAGGAGTTTATAAAAAAAAAAAAAAA
AAAAAAAAAAAAAAAAAAAAAAAAAAAAAAAAAAAGAAGAGCTTT
AGTGAGGGTTAATT
HRV-A73
(SEQ ID NO: 10)
GCGGCCGCATTTAGGTGACACTATAGTTAAAACTGGGTTTGGGTT
GTTCCCACCCAAACCACCCACGCGGTGTTGTACACTGTTATTCCG
GTAACCTTGTACGCCAGTTTTATATCCCTTCCCCCCCTTGTAACT
TAGAAGACATGCGAATCGACCAATAGCAGGCAATCAACCAGATTG
TCACCGGTCAAGCACTTCTGTTTCCCCGGCTCTCGTTGATATGCT
CCAACAGGGCAAAAACAATTGGTAGTCGTTACCCGCAAGATGCCT
ACGCAAAACCTAGTAGCATCTTCGAAGATTTTTGGTTGGTCGCTC
AGTTGCTACCCCAGCAATAGACCTGGCAGATGAGGCTAGAAATAC
CCCACTGGTGACAGTGTTCTAGCCTGCGTGGCTGCCTGCACACCC
ACACGGGTGTGAAGCCAAAGATTGGACAAGGTGTGAAGAGCACGT
GTGCTCATCTTGAAGTCCTCCGGCCCCTGAATGCGGCTAACCTTA
ACCCCGTAGCCATTGCTCGCAATCCAGCGAGTATATGGTCGTAAT
GAGTAATTACGGGATGGGACCGACTACTTTGGGTGTCCGTGTTTC
ACTTTTTACTTATCAATTTGCTTATGGTGACAATATATATAGATA
TATATTGACACCATGGGCGCCCAAGTATCTAGACAGAATGTTGGT
ACGCACTCTACACAAAACTCTGTTACAAATGGATCAAGTCTAAAT
TACTTCAACATCAACTACTTCAAGGATGCGGCATCAAGTGGAGCT
TCCAAATTAGAATTTTCACAAGACCCCAGCAAATTCACAGACCCA
GTGAAAGATGTTCTGGAGAAAGGAATTCCCACCCTACAATCTCCC
ACAGTTGAAGCGTGTGGTTATTCAGACAGGATAATCCAAATCACT
AGAGGTGATTCTACGATTACTTCACAGGATGTAGCTAATGCAGTT
GTTGCATATGGAGTTTGGCCTCATTATCTCACACCACAAGATGCA
ACTGCTATAGATAAACCAACTAGACCTGATACTTCCTCCAATAGA
TTTTATACTTTAGAAAGCCAGATGTGGTCCCCTGAATCCAAAGGA
TGGTGGTGGAAGTTACCTGATGCTTTGAAGGACATGGGCATTTTT
GGGGAAAACATGTTTTACCATTTCCTTGGGAGAAGTGGTTATACA
GTCCATGTTCAATGTAATGCCAGTAAATTTCATCAAGGAACACTA
TTAGTTGCCATGATACCAGAACATCAGTTGGCTGCCGCCAAGGGT
GGCACTGTCACAGCAGGATATAAATACACGCATCCTGGTGAAGCG
GGCAGGGATGTTGGGAAAACTGACCGCAAGCAAAACAACCGACAG
CCTAGTGATGACAATTGGCTCAATTTCGATGGTACTCTCTTAGGC
AACTCGACCATCTTCCCACACCAATTTATCAATCTCAGGAGTAAT
AATTCAGCTACTATTATAGTTCCATATGTTAATGCAGTTCCAATG
GATTCTATGCTTAGACACAATAATTGGAGTTTGGTGATTATACCT
ATATGCAAACTTAGGTGTCAAGGTATATCCCCAGTTGTACCTATC
ACAATATCTATAAGCCCTATGTGTGCTGAGTTTTCTGGAGCTAGA
GCTAGAAATGTCACTCAAGGACTGCCAACATTTATAACACCTGGA
TCTGGTCAATTCTTAACTACAGATGATTTCCAATCTCCCAGTGCA
TTGCCGTGGTATCACCCTACAAAAGAAATATCAATACCAGGACAA
GTCCGAAACCTAGTTGAACTATGTCAAGTTGATACTATGATACCA
ATCAACAATATTGCAGCAAATGTCACCAATGAGAGCATGTATGCC
ATTAGTCTTGAGAGCAATGCTGATATTAAACAAGCTGTGTTTGCA
ATGAGAGTTGACATCACCTCACAACCCTTAGCTACAACATTAATA
GGTGAGATAGCTAGTTATTACACTCATTGGACAGGGAGCCTTCGA
TTTAGTTTCATGTTTTGTGGTTCTGCATTTTCAACTCTTAAATTG
CTACTTGCTTATACACCACCAGGGATTGCTGTTCCAGATAGCAGA
AAGAAAGCTATGCTTGGTACACATGTCATATGGGATGTTGGTTTG
CAATCAACAGTATCAATAGTGGTACCTTGGGTGAGTGCTAGCCAT
TACAGAAATACAACCCCTGACACTTATTCATTAGCTGGTTATATC
ACATGCTGGTATCAAACAAAACTAGTGGAACCCCCAAACACAACC
CCAGTGGCAGACATGATATGCTTTGTTTCAGGGTGTAAGGATTTT
TGTTTACGCATGGCAAGAGATACAAATCTACATAAACAAAGTGGT
GTGATTGCACAAAATCCTGTAGAAAGATATGTAGATGAAGTTTTG
AATGAAGTCCTTGTAGTACCCAATATCAATGAAAGTAATCCGACT
ACATCCAACTCAGCACCTGCACTGGACGCTGCAGAAACTGGCCAT
ACCAGTGGTGTACAACCAGAAGACATGATTGAGACCCGTTATGTC
CAAACATCACAGACTAGAGATGAAATGAGCATTGAAAGCTTCCTT
GGCAGGTCAGGTTGTATACACATGTCAACTATGAATATAAATTAT
GAAAATTATGATGATGCTCCTGAAAATTTTACCAAATGGAAAATA
AGTTTACAAGAGATGGCTCAAATACGTAGGAAATTTGAATTATTC
ACCTATGTAAGATTTGATTCAGAAGTGACAATTGTACCATGTATA
GCTGGTCAAAGTGGAGATGTGGGACATGTAGTTATGCAGTATATG
TATGTCCCACCTGGAGCCCCTCTACCCACAAAAAGAAATGACTAC
ACATGGCAGTCCGGCACTAATGCATCAGTATTCTGGCAACATGGT
CAAACTTACCCCAGATTCTCATTACCATTCCTTAGTATAGCATCC
GCATATTATATGTTTTATGATGGATATGATGGTGATTCCGCACAA
TCACATTATGGCACCACAGTAGTTAATGACATGGGCACATTATGC
TTTAGGATAGTGACTGAAGAACACACTAGCAGGGTAAAGGTTACT
ACTAGAATCTATCATAAGGCTAAACATGTTAAAGCTTGGTGTCCA
AGACCTCCTAGGGCAGTAGAATATACAAATGCACATGTGACCAAT
TATAAACCCACTGATGGAGAAGTTACTACTGCCATTAGGCATAGA
GATAATGTTAGAGCCATCCAAAATTTTGGACCTAGTGACATGTAT
GTGCATGTTGGAAACCTAATATACAGAAATCTACACTTGTTCAAC
TCAGAGATGCATGATTCAATCTTGGTATCCTACTCATCTGATCTA
GTCATTTACCGAACAAACACTATAGGTGATGACTTTATCCCATCT
TGTGATTGCACTGAAGCTACATATTACTGTAAACACAAAAATAGA
TATTATCCAATCAAAGTCACTAGTCATGATTGGTATGAAATACAA
GAGAGTGAGTATTATCCAAAACACATACAGTACAATTTGCTAATT
GGCGAAGGTCCCTGTGAACCTGGAGATTGTGGAGGTAAACTACTA
TGTAAACATGGGGTGATTGGGATCATTACAGCTGGTGGTGAGGGC
CATGTTGCTTTCATTGATTTGAGACATTTCTTATGTGCTGAGGAA
CAAGGTGTTACTGACTATATACATATGTTAGGTGAAGCTTTTGGC
AATGGTTTTGTAGATAGTGTTAAAGAACATGTTAATGCAATAAAC
CCTATTAATAGTATTAGTAAAAAAGTGATTAAATGGCTACTGAGG
ATTGTATCCGCTATGGTCATTATCATTAGAAATTCTTCAGATCCA
CATACTGTCATTGCTACTCTAACATTAATTGGTTGTTCTGGGTCG
CCTTGGAGGTTCTTGAAAGAGAAATTTTGCAAATGGACCCAGTTA
AATTATATCCATAAAGAATCTGATTCATGGCTAAAGAAATTTACT
GAAATGTGTAATGCAGCGAGAGGTCTTGAGTGGATAGGCAATAAG
ATATCTAAGTTTATAGAATGGATGAAATCAATGTTACCACAAGCA
CAACTCAAAGTTAAGTATTTGAATGAGTTAAAGAAATTGAGCTTA
CTTGAAAAACAGGTTGAAAACTTAAGATGTGCCGACACTAAGACA
CAAGAGAAGATAAAAGTTGAGATAGACACCCTGCATGATTTATCT
TGTAAATTTCTCCCATTATATGCCAGTGAGGCTAAAAGAATCAAA
GTTATTTATAACAAATGCAATAATATCATCAAGCAAAAGAAAAGA
AGTGAACCTGTTGCAGTAATGATACATGGTTCACCTGGGACTGGC
AAATCTATCACAACAAGCTTTCTGGCAAGAATGATTACTAATGAA
AGTGACATATACTCTTTACCTCCTGACCCAAAGTACTTTGATGGC
TATGATCAGCAAAGTGTCGTTATAATGGATGACATTATGCAAAAT
CCTGACGGTGAGGACATGTCACTGTTTTGCCAAATGGTTTCCAGT
GTGACCTTCATTCCACCAATGGCTGATTTGCCAGACAAGGGGAAA
CCCTTTGATTCAAGATTTGTTCTATGTAGCACTAACCACTCTCTC
TTAGCTCCTCCCACCATCACCTCTCTTCAGGCAATGAATAGGCGG
TTCTTCTTAGACTTAGATATTGTGGTCCATGATAATTACAAAGAT
ACACAAGGCAAGTTGGATGTGTCCAAAGCATTCAAACCATGTGAT
GTTGGGACAAAAATAGGAAATGCCCGCTGTTGCCCATTTATATGT
GGCAAAGCAGTAACCTTCAAAGACCGCAATACTTGCCTGAGTTAT
TCATTGAGCCAAATTTACAACTTGATTCTTCAGGAAGACAAAAGG
CGCACCCATGTTGTGGACGTCATGACTGCTATATTTCAAGGACCA
ATTTCAATGGAAGTCCCCCCCCCTCCTGCTATAACTGATCTCTTA
CGCTCTGTCAGAACACCAGAAGTTATTAAATACTGTGAAAACAAC
AAATGGATCGTTCCAGCTGACTGCAAAATTGAGCGAGATTTGAAC
CTTGCAAATAATATCATAACAATCATTGCTAATATAATTAGTATA
GCAGGCATCATATACATTATATACAAACTGTTCTGCTCTTTTCAA
GGACCCTATTCAGGTGAACCCAAACCAAAAACAAAAGTCCCAGAG
AGGAGGGTGGTTGCACAAGGTCCGGAGGAAGAATTTGGCCGCTCT
CTAATCAAACACAACACATGTGTAGTCACTACTGATAATGGTAAA
TTTACTGGGCTGGGAATCTATGACAAACTTATGATATTACCCACA
CATGCTGATCCTGGTAAGGAGGTATATATCAATGGGATTGCAACC
AAAGTTAGTGATTCATATGACTTGTATAATAAACAGGGTGTTAAA
TTAGAGATTACAGCTATAGTGCTAGATAGAAATGAAAAGTTCAGG
GATATACGCAAGTATATACCGGAAAGGGAGGATGATTACCCTGAT
TGCAACTTAGCCCTTGTAGCCAATCAATCTGAACCCACTATTATA
AATGTAGGGGATGTGATCTCCTATGGTAACATCTTGCTCAGTGGC
AATCAAACGGCACGAATGCTTAAATATAATTATCCCACAAAATCA
GGGTATTGTGGTGGTGTTCTATATAAAATAGGCCAAATTATAGGA
ATCCATGTGGGTGGTAATGGCAGGGATGGATTTTCTGCAATGTTA
CTCAGATCTTACTTCAATCAAACTCAAGGAGAAATAACAGTGACA
AAGAAGGTGGCTGAATGTGGACTTCCAACGATACACACTCCGTCC
AAAACTAAGCTACAACCTAGTGTCTTCTTTGATGTTTTTGAAGGA
TCCAAAGAACCAGCTGTCCTAACTGACAAAGACCCCCGATTAACT
ACTGATTTTAATAAAGCTCTATTTTCCAAATACAAGGGTAATATA
GAATGTAGCATGACTGATCATATGAGGGTTGCTATCTCACACTAT
TCAGCTCAATTAATGACCCTGGACATTGATTCAACAAATATGTCC
CTAGAGGAGAGTGTATTTGGCACTGAAGGCTTGGAGGCTCTTGAT
CTAAATACAAGTGCAGGCTTTCCATATATAAGTATGGGAATTAGG
AAAAAAGATCTAATTAATAACAGTACAAAGGACATAACTAAATTG
AAGCTAGCACTTGATAAATATGGGGTTGATTTACCAATGGTTACA
TTCCTCAAAGATGAACTTAGAAAGAAGGAGAAAATATCCACTGGC
AAAACAAGAGTGATTGAAGCTAGTAGTGTGAATGATACAATAGCT
TTTAGGGTAGTGTATGGAAATTTGTTTTCTACTTTTCACAAGAAC
CCTGGTGTAGTCACAGGATCTGCGGTGGGATGTGACCCTGAGACC
TTTTGGTCAAAGATACCAGTGATGCTAGATGGGGAATGTATAATG
GCATTTGATTACACCAATTATGATGGTAGTATACACCCAATTTGG
TTTGAAGCTCTAAAACAAGTTCTAAACAATTTATCATTTGAAGAC
AAGCTTATAGATAGATTGTGTAGATCAAAACATATATTTAGAGAC
ACATACTATGAAGTTGAAGGTGGGGTGCCCTCAGGTTGTTCAGGA
ACTAGCATATTTAATACTATGATAAATAACATCATTATTAGGACT
TTAGTTCTTGATGCTTATAAGCATATTGACTTGGACAAACTTAAA
ATAATAGCTTATGGTGATGATGTTATTTTCTCTTATAAGTATCCC
TTAGACATGGAAGCAATTGCTGTAGAAGGAAATAAGTACGGTTTG
ACCATTACACCGGCTGATAAATCAGATACTTTTAGGAAGCTTGAT
TATGACAATGTAACTTTCCTGAAAAGAGGTTTTAGGCAAGATGCT
AAATATCCATTCCTAATTCATCCTACATTTCCTGTTTCTGAGATT
CATGAATCAATTAGATGGACCAAGAAACCCTCACAGATGCAGGAA
CATGTTTTATCCTTGTGTCATTTAATGTGGCACAATGGACGGGAT
GTGTACAAGGAGTTTGAGAGGAAGATACGCAGCGTGAGCGCTGGA
CGCGCGCTGTACATCCCCCCTTATGAGCTACTCCTGCATGAGTGG
TATGAAAAATTTTAAATTATATATAGAAATAATAAACATTTAGTT
TTTTAGTTTTACAAAAAAAAAAAAAAAAAAAAAAAAAGACTCTCG
AG

### 1. Initial Infectious HRV Generation

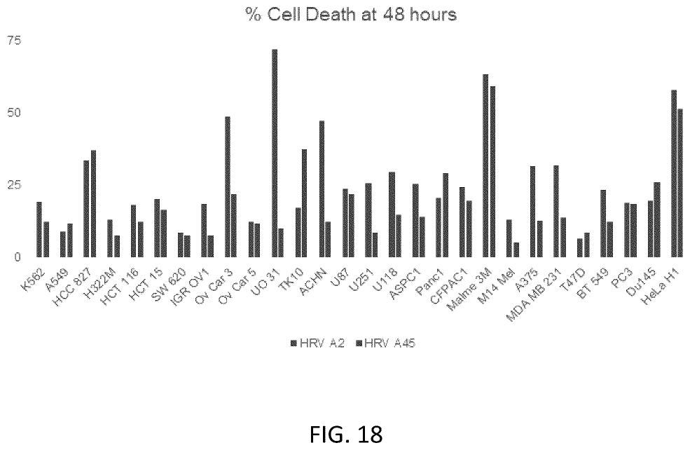

To generate infectious virus in vitro RNA transcription of each synthetic DNA template was initiated via an engineered upstream phage promoter sequence (e.g., T7, SP6) (FIGS. 18A and 18B) followed by standard lipid mediated transfection (Lipofectamine 2000) in HELA-H1 cells. 48 hours following transfection, infectious virus was isolated by four crude freeze/thaw cycles. Following brief centrifugation to remove cellular debris, crude viral stocks of unknown titer were then used to infect HELA-H1 cells in suspension for amplification. 24 hours post infection cells were harvested, underwent three rapid freeze/thaw cycles, and supernatant containing virus was subjected to ultracentrifugation through a 30% sucrose gradient at 107,000×g (25,000 RPM, SW40 rotor) for four hours. Pelleted virus was then reconstituted in Tris-PBS buffer, aliquoted, and stored at −80 C. This primary stock was then titered in HELA-H1 cells using standard dilution procedures and quantified by RT-PCR using specific primers for each HRV serotype. Subsequent amplification steps were carried out in a similar manner but with varying multiplicity of infection (MOI) ranging from 0.1 to 10 in order to generate the highest viral stocks. Shown in (FIG. 18C) is the titer of HRV-A2 following two rounds of amplification. These viral stocks ranging from 10⁷-10⁸ infectious units per mL (IFU/mL) were then used to generate higher titer viral stocks and infect various human and murine cancer cell lines as well as normal primary human cells.

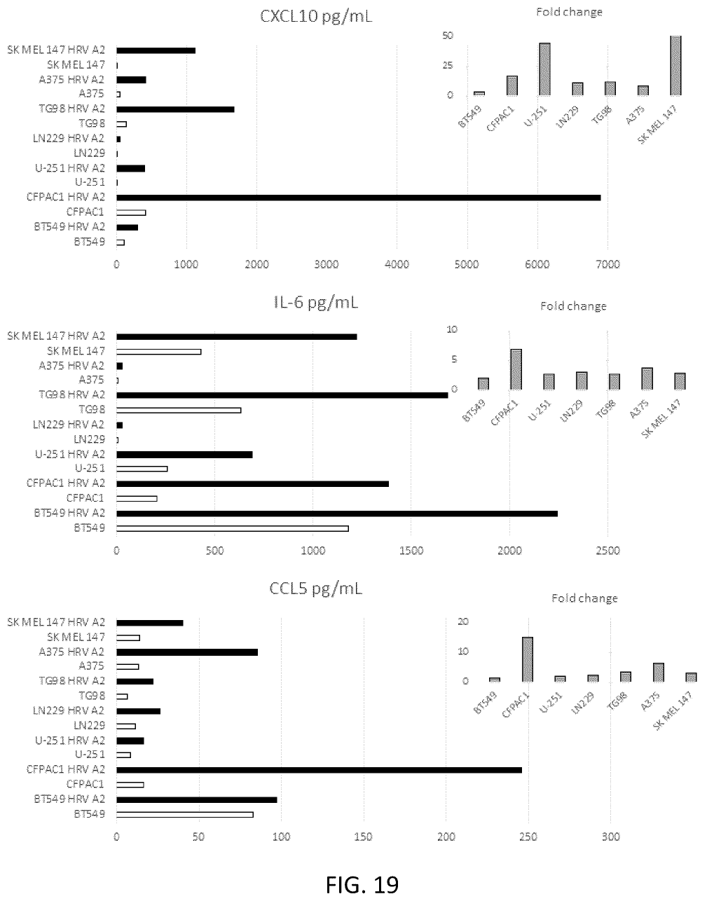

The expression of ICAM1 in melanoma cell lines was evaluated and the ability of HRV-A45 to infect these human lines was assessed. Varying levels of ICAM1 expression were detected in a panel of human melanoma cell lines by Western blot (FIG. 19A). Efficient infection of each human melanoma cell line except CHL-1 cells (FIG. 19C) which lack detectable ICAM1 expression was observed. Importantly, HRV-A45 was able to efficiently infect murine melanomas expressing human ICAM1 (FIG. 19B). ICAM1 expression and susceptibility to HRV-A45 infection was evaluated in a panel of glioblastoma and breast cancer cell lines and similar results observed (FIG. 20). While ICAM1 expression is required for HRV-A45 infection, receptor expression was marginally predictive of susceptibility indicating additional factors impact viral replication.

Similar results were obtained with ICAM1 selective HRVs (HRV-B14, HRV-A73) as expected. ICAM1 and LDLR expression were assessed in a large diverse panel of human cancer cell lines. ICAM1 was found to be expressed at detectable levels in nearly all cell lines examined. HRV-A2, HRV-A23, and HRV-A25 each utilize members of the LDLR family of receptors making it quite difficult to predict infectivity based on expression of LDLR alone. Generally, these LDLR-restricted viruses infect most human cancer cell lines efficiently with limited capacity to infect some murine and canine tumor cells indicating that one or more non-human LDLR family members are sufficient to confer susceptibility. Dose dependent cell death was observed following infection with HRV-A2 in some canine cancer cells as well as cell death in human CHL-1 cells that lack ICAM1 (FIG. 21). Even at high MOI, ICAM1-selective HRV-A45 has negligible effect on CHL-1 cells and canine cells (FIG. 21), further demonstrating the impactful role of each receptor in viral tropism.

The analysis of HRV oncolysis was expanded to include diverse human cancer cell lines encompassing melanoma, hematopoietic, lung, colon, ovarian, breast, renal, prostate, and pancreatic cancer (FIG. 22). Significant and variable cell death following low MOI infection was observed at 48 hours.

Like other RNA viruses, HRVs elicit a rapid innate immune response following infection. 45 immunomodulatory chemokines and cytokines were assessed from numerous human cancer cell lines 24 hours following low MOI in vitro infection. Dramatic increases in multiple interferon (IFN) response genes including CXCL10, IL-6, and CCL5 after HRV infection were seen (FIG. 23). Each of these soluble mediators are involved in recruiting and activating multiple immune cell subsets including CD8+ T cells demonstrating that in addition to direct tumor cell oncolysis, HRVs are able to impact immune cellular infiltration.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Eissa, I. R., et al., The Current Status and Future Prospects of Oncolytic Viruses in Clinical Trials against Melanoma, Glioma, Pancreatic, and Breast Cancers. Cancers (Basel), 2018. 10(10).
2. Kelly, E. and S. J. Russell, History of oncolytic viruses: genesis to genetic engineering. Mol Ther, 2007. 15(4): p. 651-9.
3. Dock, G., The influence of complicating diseases upon leuk ae mia. American Journal of the Medical Sciences, 1904. 127: p. 563-592.
4. Martuza, R. L., et al., Experimental therapy of human glioma by means of a genetically engineered virus mutant. Science, 1991. 252(5007): p. 854-6.
5. Andtbacka, R. H., et al., Talimogene Laherparepvec Improves Durable Response Rate in Patients With Advanced Melanoma. J Clin Oncol, 2015. 33(25): p. 2780-8.
6. Chen, C. Y., et al., Oncolytic virus and PD-1/PD-L1 blockade combination therapy. Oncolytic Virother, 2018. 7: p. 65-77.
7. Ribas, A., et al., Oncolytic Virotherapy Promotes Intratumoral T Cell Infiltration and Improves Anti-PD-1 Immunotherapy. Cell, 2017. 170(6): p. 1109-1119 e10.
8. Perez, M. C., et al., Talimogene Laherparepvec (TVEC) for the Treatment of Advanced Melanoma: A Single-Institution Experience. Ann Surg Oncol, 2018. 25(13): p. 3960-3965.
9. He, B., et al., Suppression of the phenotype of gamma(1)34.5-herpes simplex virus 1: failure of activated RNA-dependent protein kinase to shut off protein synthesis is associated with a deletion in the domain of the alpha47 gene. J Virol, 1997. 71(8): p. 6049-54.
10. Chou, J. and B. Roizman, The gamma 1(34.5) gene of herpes simplex virus 1 precludes neuroblastoma cells from triggering total shutoff of protein synthesis characteristic of programed cell death in neuronal cells. Proc Natl Acad Sci USA, 1992. 89(8): p. 3266-70.
11. Liu, B. L., et al., ICP34.5 deleted herpes simplex virus with enhanced oncolytic, immune stimulating, and anti-tumour properties. Gene Ther, 2003. 10(4): p. 292-303.
12. Brown, S. M., et al., The herpes simplex virus virulence factor ICP34.5 and the cellular protein MyD116 complex with proliferating cell nuclear antigen through the 63-amino-acid domain conserved in ICP34.5, MyD116, and GADD34. J Virol, 1997. 71(12): p. 9442-9.
13. Goldsmith, K., et al., Infected cell protein (ICP)47 enhances herpes simplex virus neurovirulence by blocking the CD8+ T cell response. J Exp Med, 1998. 187(3): p. 341-8.
14. Poppers, J., et al., Inhibition of PKR activation by the proline-rich RNA binding domain of the herpes simplex virus type 1 Us11 protein. J Virol, 2000. 74(23): p. 11215-21.
15. Kaufman, H. L., et al., Current status of granulocyte-macrophage colony-stimulating factor in the immunotherapy of melanoma. J Immunother Cancer, 2014. 2: p. 11.
16. Lundstrom, K., New frontiers in oncolytic viruses: optimizing and selecting for virus strains with improved efficacy. Biologics, 2018. 12: p. 43-60.
17. Jacobs, S. E., et al., Human rhinoviruses. Clin Microbiol Rev, 2013. 26(1): p. 135-62.
18. Koch, R. M., et al., Short-term repeated HRV-16 exposure results in an attenuated immune response in vivo in humans. PLOS One, 2018. 13(2): p. e0191937.
19. Lee, S., et al., A polyvalent inactivated rhinovirus vaccine is broadly immunogenic in rhesus macaques. Nat Commun, 2016. 7: p. 12838.
20. Glanville, N., et al., Cross-serotype immunity induced by immunization with a conserved rhinovirus capsid protein. PLOS Pathog, 2013. 9(9): p. e1003669.
21. Hak Kim, J.-H. N., Human Rhinoviruses: the Forgotten but Still Important Viruses. Journal of Bacteriology and Virology, 2017. 47: p. 111-121.
22. Sanjuan, R., et al., Viral mutation rates. J Virol, 2010. 84(19): p. 9733-48.
23. Lauring, A. S., J. Frydman, and R. Andino, The role of mutational robustness in RNA virus evolution. Nat Rev Microbiol, 2013. 11(5): p. 327-36.
24. Perez-Losada, M., et al., Recombination in viruses: mechanisms, methods of study, and evolutionary consequences. Infect Genet Evol, 2015. 30: p. 296-307.
25. Dotzauer, A. and L. Kraemer, Innate and adaptive immune responses against picornaviruses and their counteractions: An overview. World J Virol, 2012. 1(3): p. 91-107.
26. VanBrocklin, M. W., et al., Mitogen-activated protein kinase inhibition induces translocation of Bmf to promote apoptosis in melanoma. Cancer Res, 2009. 69(5): p. 1985-94.
27. Meeth, K., et al., The YUMM lines: a series of congenic mouse melanoma cell lines with defined genetic alterations. Pigment Cell Melanoma Res, 2016. 29(5): p. 590-7.
28. Dufresne, A. T. and M. Gromeier, A nonpolio enterovirus with respiratory tropism causes poliomyelitis in intercellular adhesion molecule 1 transgenic mice. Proc Natl Acad Sci USA, 2004. 101(37): p. 13636-41.
29. Wang, E. S., et al., Adaptation of an ICAM-1-tropic enterovirus to the mouse respiratory tract. J Virol, 2011. 85(11): p. 5606-17.
30. Eisenhauer, E. A., et al., New response evaluation criteria in solid tumours: revised RECIST guideline (version 1.1). Eur J Cancer, 2009. 45(2): p. 228-47.
31. Varedi, A., et al., ASA Suppresses PGE2 in Plasma and Melanocytic Nevi of Human Subjects at Increased Risk for Melanoma. Pharmaceuticals (Basel), 2020. 13(1).
32. Burnett, W. J., et al., Prior Exposure to Coxsackievirus A21 Does Not Mitigate Oncolytic T herapeutic Efficacy. Cancers (Basel), 2021. 13(17).
33. Corrigan, P. A., C. Beaulieu, R. B. Patel, and D. K. Lowe. 2017. ‘Talimogene Laherparepvec: An Oncolytic Virus Therapy for Melanoma’, Ann Pharmacother, 51: 675-81.
34. Liang, M. 2018. ‘Oncorine, the World First Oncolytic Virus Medicine and its Update in China’, Curr Cancer Drug Targets, 18: 171-76.
35. Siegel, R. L., K. D. Miller, and A. Jemal. 2018. ‘Cancer statistics, 2018’, CA Cancer J Clin, 68: 7-30.
36. Siegel, R. L., Miller, K. D. & Jemal, A. Cancer statistics, 2019. CA Cancer J Clin 69, 7-34, doi: 10.3322/caac.21551 (2019).
37. Hulbert, M. Melanoma Mortality Rates Decreasing Despite Ongoing Increase in Incidence Rates, 2019).
38. Cancer.Net. Melanoma: Statistics, 2019).
39. Farkona, S., Diamandis, E. P. & Blasutig, I. M. Cancer immunotherapy: the beginning of the end of cancer? BMC Med 14, 73, doi: 10.1186/s12916-016-0623-5 (2016).
30. Christiansen, S. A., Khan, S. & Gibney, G. T. Targeted Therapies in Combination With Immune Therapies for the Treatment of Metastatic Melanoma. Cancer J 23, 59-62, doi: 10.1097/PPO.0000000000000245 (2017).
41. Andtbacka, R. H. The role of talimogene laherparepvec (T-VEC) in the age of checkpoint inhibitors. Clin Adv Hematol Oncol 14, 576-579 (2016).
42. Andtbacka, R. H. et al. Talimogene Laherparepvec Improves Durable Response Rate in Patients With Advanced Melanoma. J Clin Oncol 33, 2780-2788, doi: 10.1200/JCO.2014.58.3377 (2015).
43. Kohlhapp, F. J. & Kaufman, H. L. Molecular Pathways: Mechanism of Action for Talimogene Laherparepvec, a New Oncolytic Virus Immunotherapy. Clin Cancer Res 22, 1048-1054, doi: 10.1158/1078-0432.CCR-15-2667 (2016).
44. Clemens, M. J. Targets and mechanisms for the regulation of translation in malignant transformation. Oncogene 23, 3180-3188, doi: 10.1038/sj.onc.1207544 (2004).
45. Smith, K. D. et al. Activated MEK suppresses activation of PKR and enables efficient replication and in vivo oncolysis by Deltagamma(1)34.5 mutants of herpes simplex virus 1. J Virol 80, 1110-1120, doi: 10.1128/JVI.80.3.1110-1120.2006 (2006).
46. Puzanov, I. et al. Talimogene Laherparepvec in Combination With Ipilimumab in Previously Untreated, Unresectable Stage IIIB-IV Melanoma. J Clin Oncol 34, 2619-2626, doi: 10.1200/JCO.2016.67.1529 (2016).
47. Ribas, A. et al. Oncolytic Virotherapy Promotes Intratumoral T Cell Infiltration and Improves Anti-PD-1 Immunotherapy. Cell 170, 1109-1119 e1110, doi: 10.1016/j.cell.2017.08.027 (2017).
48. Jacobs, S. E. et al. Human rhinovirus infections of the lower respiratory tract in hematopoietic stem cell transplant recipients. Transpl Infect Dis 15, 474-486, doi: 10.1111/tid. 12111 (2013).
49. Palmenberg, A. C. et al. Sequencing and analyses of all known human rhinovirus genomes reveal structure and evolution. Science 324, 55-59, doi: 10.1126/science.1165557 (2009).
50. Stobart, C. C., Nosek, J. M. & Moore, M. L. Rhinovirus Biology, Antigenic Diversity, and Advancements in the Design of a Human Rhinovirus Vaccine. Front Microbiol 8, 2412, doi: 10.3389/fmicb.2017.02412 (2017).
51. McIntyre, C. L., Knowles, N. J. & Simmonds, P. Proposals for the classification of human rhinovirus species A, B and C into genotypically assigned types. J Gen Virol 94, 1791-1806, doi: 10.1099/vir.0.053686-0 (2013).
52. Palmenberg, A. C. Rhinovirus C, Asthma, and Cell Surface Expression of Virus Receptor CDHR3. J Virol 91, doi: 10.1128/JVI.00072-17 (2017).
53. Min, I. M. et al. CAR T Therapy Targeting ICAM-1 Eliminates Advanced Human Thyroid Tumors. Clin Cancer Res 23, 7569-7583, doi: 10.1158/1078-0432.CCR-17-2008 (2017).
54. Roslan, Z., Muhamad, M., Selvaratnam, L. & Ab-Rahim, S. The Roles of Low-Density Lipoprotein Receptor-Related Proteins 5, 6, and 8 in Cancer: A Review. J Oncol 2019, 4536302, doi: 10.1155/2019/4536302 (2019).
55. McIntyre, C. L., McWilliam Leitch, E. C., Savolainen-Kopra, C., Hovi, T. & Simmonds, P. Analysis of genetic diversity and sites of recombination in human rhinovirus species C. J Virol 84, 10297-10310, doi: 10.1128/JVI.00962-10 (2010).
56. McIntyre, C. L., Savolainen-Kopra, C., Hovi, T. & Simmonds, P. Recombination in the evolution of human rhinovirus genomes. Arch Virol 158, 1497-1515, doi: 10.1007/s00705-013-1634-6 (2013).
57. Winther, B. Rhinovirus infections in the upper airway. Proc Am Thorac Soc 8, 79-89, doi: 10.1513/pats.201006-039RN (2011).
58. Andtbacka, R. H. et al. CALM Study: A Phase II Study of a Novel Oncolytic Immunotherapeutic Agent, CVA21, Delivered Intratumorally in Patients with Advanced Malignant Melanoma. Annals of Surgical Oncology 22, S22-S22 (2015).
59. Andtbacka, R. H. I. et al. Intralesional administration of Coxsackievirus A21 (CVA21) oncolytic virus immunotherapy in patients with advanced melanoma induces durable tumor responses. Eur J Cancer 51, S676-S677 (2015).
60. Andtbacka, R. H. I. et al. Final data from CALM: A phase II study of Coxsackievirus A21 (CVA21) oncolytic virus immunotherapy in patients with advanced melanoma. J Clin Oncol 33 (2015).
61. Kaufman, H. L. et al. Phase Ib study of intratumoral oncolytic coxsackievirus A21 (CVA21) and pembrolizumab in subjects with advanced melanoma. Ann Oncol 27, doi: 10.1093/annonc/mdw379.51 (2016).
62. Rudin, C. M. et al. Phase Ib KEYNOTE-200: A study of an intravenously delivered oncolytic virus, coxsackievirus A21 in combination with pembrolizumab in advanced NSCLC and bladder cancer patients. Ann Oncol 29 (2018).
63. Silk, A. et al. CAPRA: A Phase 1b study of intratumoral Coxsackievirus A21 (CVA21) and systemic pembrolizumab in advanced melanoma patients. J Immunother Cancer 5 (2017).
64. Silk, A. W. et al. Phase 1b study of intratumoral Coxsackievirus A21 (CVA21) and systemic pembrolizumab in advanced melanoma patients: Interim results of the CAPRA clinical trial. Cancer Res 77, doi: 10.1158/1538-7445.Am2017-Ct026 (2017).
65. Wong, Y. V. Y., Quah, M. Y., Shafren, D. R. & Au, G. G. Synergistic Activity of Coxsackievirus A21 (CVA21) and Docetaxel in Non-Small Cell Lung Cancer (NSCLC). Hum Gene Ther 25, A28-A29 (2014).
66. Pandha, H. et al. Phase 1b KEYNOTE 200 (STORM study): A study of an intravenously delivered oncolytic virus, Coxsackievirus A21 in combination with pembrolizumab in advanced cancer patients. Cancer Res 77, doi: 10.1158/1538-7445.Am2017-Ct115 (2017).
67. Pandha, H. S. et al. Phase I STORM study (KEYNOTE 200): Intravenous delivery of a novel oncolytic immunotherapy agent, Coxsackievirus A21 in combination with pembrolizumab in advanced cancer patients. J Clin Oncol 34, doi:10.1200/JCO.2016.34.15_suppl.TPS3108 (2016).
68. Desjardins, A. et al. Recurrent Glioblastoma Treated with Recombinant Poliovirus. N Engl J Med 379, 150-161, doi: 10.1056/NEJMoa1716435 (2018).
69. Gao, J., Zheng, Q., Xin, N., Wang, W. & Zhao, C. CD155, an onco-immunologic molecule in human tumors. Cancer Sci 108, 1934-1938, doi: 10.1111/cas.13324 (2017).

1. A human rhinovirus comprising one or more single nucleotide polymorphisms (SNP), wherein the SNPs are in the nucleic acid sequences in the 5′UTR, VP4, VP3, VP1, P2-C polypeptide, P3-A polypeptide, VpG protein, Protease 3C, or RDRP regions when compared to the nucleic acid sequence that is available under the GenBank accession number X02316.1.

2. The human rhinovirus of claim 1, wherein the one or SNPs are A228U, U491C, C740A, A744G, A1735G, A2491U, G2517A, G2714, GC4755/4756CG, A4854G, G5018A, G5023A, GA5026/5027AG, G5152A, A5300G, C5521U, or A6238G when compared to the nucleic acid sequence that is available under the GenBank accession number X02316.1.

3. The human rhinovirus of claim 1, wherein the human rhinovirus comprises the SNPs A228U, U491C, C740A, A744G, A1735G, A2491U, G2517A, G2714A, GC4755/4756CG, A4854G, G5018A, G5023A, GA5026/5027AG, G5152A, A5300G, C5521U, and A6238G when compared to the nucleic acid sequence that is available under the GenBank accession number X02316.1.

4. The human rhinovirus of claim 1, wherein the human rhinovirus is encoded by the nucleic acid sequence of SEQ ID NO:1.

5. The human rhinovirus of claim 1, wherein the human rhinovirus comprises the amino acid sequence of SEQ ID NO:2.

6. A human rhinovirus comprising one or more single nucleotide polymorphisms (SNP), wherein the SNPs are in the nucleic acid sequences in the VP3, VP1, P2-B polypeptide, P2-C polypeptide, or P3-A polypeptide regions when compared to the nucleic acid sequence that is available under the GenBank accession number FJ445132.1.

7. The human rhinovirus of claim 6, wherein the one or SNPs are A1665U, A1746G, G2953C, U3262C, G3631A, A3817G, A4007G, U4020C, A4194G, A4664G, U4695C, A4917G, or U,4952 when compared to the nucleic acid sequence that is available under the GenBank accession number FJ445132.1.

8. The human rhinovirus of claim 6, wherein the human rhinovirus comprises the SNPs A1665U, A1746G, G2953C, U3262C, G3631A, A3817G, A4007G, U4020C, A4194G, A4664G, U4695C, A4917G, and U,4952 when compared to the nucleic acid sequence that is available under the GenBank accession number FJ445132.1.

9. The human rhinovirus of claim 6, wherein the human rhinovirus is encoded by the nucleic acid sequence of SEQ ID NO:3.

10. The human rhinovirus of claim 6, wherein the human rhinovirus comprises the amino acid sequence of SEQ ID NO:4.

11. A composition comprising the human rhinoviruses of claim 1.

12.-85. (canceled)

86. A composition comprising the human rhinoviruses of claim 6.

87. A method of treating cancer in a subject comprising: administering the composition of claim 11 to the subject.

88. A method of treating cancer in a subject comprising: administering the composition of claim 86 to the subject.

89. A method of treating a viral induced lesion of the skin in a subject comprising: administering the composition of claim 11 to the subject.

90. A method of treating a viral induced lesion of the skin in a subject comprising: administering the composition of claim 86 to the subject.


SEQUENCE LISTING (10 sequences)

SEQ ID NO: 1 - DNA, 7102 bp, synthetic construct
  source 1..7102: mol_type = other DNA
ttaaaactggatccaggttgttcccacctggatttcccacagggagtggtactctgttattacggtaactttgtacgccagttttatctcccttcccccatgtaacttagaagtttttcacaaagaccaatagccggtaatcagccagattactgaaggtcaagcacttctgtttccccggtcaatgttgatatgctccaacagggcaaaaacaactgcgatcgttatccgcaaagcgcctacgcaaagcttagtagcatctttgaaatcgtttggctggtcgatccgccatttcccctggtagacctggcagatgaggctagaaataccccactggcgacagtgttctagcctgcgtggctgcctgcacaccctatgggtgtgaagccaaacaatggacaaggtgtgaagagccccgtgtgctcgctttgagtcctccggcccctgaatgtggctaaccttaaccctgcagctagagcacgtaacccaacgtgtatctagtcgtaatgagcaattgcgggatgggaccaactactttgggtgtccgtgtttcactttttcctttatatttgcttatggtgacaatatatacaatatatatattggcaccatgggtgcacaggtttcaagacaaaatgttggaactcactccacgcaaaactctgtatcaaatgggtctagtttaaattattttaacatcaattatttcaaagatgctgcttcaaatggtgcatcaaaaatgggattcacacaagatcctagtaaatttactgacccagttaaggatgttttggaaaagggaataccaacactacagtcccccacagtggaggcttgtggatactctgataggattatacagattaccagaggagattcaaccataacctcacaagatgtggctaatgctatcgttgcgtatggtgtttggccacattatctatcctccaaggatgcctctgcaattgataaaccctctcaaccagatacatcttctaatagattttatactctaaggagtgtgacctggagcagttcctcaaagggttggtggtggaaactacctgatgcactcaaggacatgggtatttttggtgaaaacatgttttatcattacctgggtaggagtggatacacaatacatgtgcagtgtaatgctagtaaatttcaccagggtacactaattgttgctctgatacctgagcatcagattgcaagtgccttacatggcaatgtgaatgttggttacaactacacacacccaggtgaaacaggcagggaagttaaagctgagacgagattgaatcctgatctacaacctactgaagagtattggctaaactttgatgggacactccttggaaatattaccatattccctcatcaatttatcaacttgaggagtaataattctgccacaataattgccccttatgtcaatgcagttcctatggattcaatgcggagccacaataattggagtttggtaataataccaatatgtccccttgagacatcaagtgcaattaacacaatacctattacaatatctataagccccatgtgtgcagagttttccggcgcgcgtgccaagcgtcaaggattaccagttttcatcacaccaggttcaggacagtttttgacaacagatgatttccaatccccatgtgcacttccctggtatcacccaactaaggaaatttctattccaggtgaggttaaaaatttggttgagatttgtcaagtagacagcctagtaccaataaataacactgacacctacatcaatagtgaaaatatgtattctgttgtattgcaatcatcaattaatgcaccagataagatcttctctattcgaacagatgttgcttcccaacctttagctactactttgattggtgagatatctagctatttcacccactggacagggagtctccgtttcagcttcatgttttgtggtactgccaacactactgttaagcttttgttggcatacacaccacctggtatcgcagaacccaccacaagaaaggatgcaatgctaggcactcatgttatatgggatgtggggttgcagtctacaatatcaatggtagtgccatggattagcgctagtcattatagaaacacatcaccaggtagatctacatctgggtacataacatgctggtatcagactagattagtcattccacctcagaccccaccaacagctagattgttatgttttgtatctgggtgcaaagacttttgcttgcgcatggcacgagatactaacctacacctgcaaagtggtgcaatagcacagaaccctgttgagaattatatagatgaagttcttaatgaagttttagttgtcccaaatattaatagtagtaaccccacaacatcaaattctgccccagcattagatgctgcagaaacagggcacactagtagtgttcaaccagaggatgtcattgaaactaggtatgtgcagacatcacatacaagagatgaaatgagtttagagaattttcttggcagatcaggatgcatacatgaatctaaattagaggttacacttgcaaattataacaaggagaattttacagtgtgggctattaatctacaagaaatggctcaaattagaaggaaatttgaattgttcacctatactaggtttgattctgaaataaccctagttccatgcatttccgcccttagtcagaacattggacacatcacaatgcaatacatgtatgttccaccaggtgcaccggtgcccaatagtagggacgattatgcatggcagtctggcactaatgcctctgttttctggcaacatggacaggcttatccaagattttccttacctttcctaagtgtggcatctgcttattacatgttttatgatgggtatgatgaacaagatcaaaactatggtacagcaaacacaaataacatggggtcactatgctctaggatagtaacagagaaacacattcataaagtacatataatgacaagaatctatcacaaggctaaacatgtcaaggcatggtgtccacgcccacccagagcgcttgagtatactcgtgctcatcgcactaattttaaaattgaggataggagtattcagacagcaattgtgaccagaccaattatcactacagctggccccagtgacatgtatgttcatgtaggtaaccttatttatagaaatcttcatcttttcaactctgagatgcatgaatctattttggtatcttattcatcagatttaatcatttaccgaacaaacactgtaggtgatgattacattccctcttgtgattgtacccaagctacttattattgcaaacataaaaatagatacttcccaattacagttacaagccatgactggtatgaaatacaggaaagtgagtactatcccaaacacatacagtacaatttgttgattggtgagggcccttgtgaaccaggtgactgtggtggaaagttgctatgcaaacatggtgtcataggtatagtaacagctggtggtgataatcatgtggcttttattgaccttagacacttccattgtgctgaagaacaaggggttacagattatatacatatgctaggagaagcatttggaaatggatttgtggatagtgtaaaagaacatatacatgccataaacccagtaggaaatatcagcaagaaaattattaaatggatgttgagaataatatcagcaatggtcataataattagaaactcttctgacccccaaactatattagcaacactcacactgattgggtgttctggatcaccctggagatttttaaaggaaaaattctgtaaatggacacagcttaattatatacacaaagaatcagattcatggttaaagaaatttactgaagcatgcaatgcagctagagggcttgaatggatagggaataagatatctaaatttattgaatggatgaagtcgatgctcccgcaagctcaattgaaggttaagtacttaaacgagcttaaaaaactcaacctatacgaaaagcaagttgagagcttgcgggtggctgacatgaaaacacaagaaaaaattaaaatggaaatagacactttacatgatttgtcacgtaaatttctacctttgtatgcaagtgaggcaaaaaggataaaaaccctatacattaaatgtgataatatcatcaagcagaagaaaagatgtgaaccagtagctatagttattcatggaccacctggtgctggcaaatctataacaacaaatttcctggccaaaatgataactaatgatagtgacatatactctctacctcctgatccaaaatattttgatggttatgaccaacagagtgtagtaataatggatgacattatgcagaatccagccggggatgacatgacactgttctgccaaatggtttctagtgttacatttataccaccaatggctgatctaccagataaaggcaaggcttttgattctaggtttgtattatgcagcacaaatcattcccttctaacacccccgacaataacttcactacctgcaatgaatagaagatttttcctagatttagatataatagtacatgataacttcaaagatccacagggcaaacttaatgtggcagcagcgtttcgaccatgtgatgtagataatagaataggaaatgcacgttgttgtccatttgtgtgtggaaaagcagtttctttcaaagatcgtaactcttgcaacaaatacacgcttgcgcaggtgtacaacataatgattgaagaagacagacggagaagacaagtggttgatgtcatgacagctatattccaagggccaattgatatgagaaacccaccaccacctgctattactgacttgctccagtctgttagaacccctgaagttattaagtattgtgagggtaatagatggataattccagcagaatgcaagatagaaaaggagttgaacttggctaacacaatcataacaatcattgcaaatgttattagtatgacaggaataatatatgttatttacaaacttttttgcacattacagggaccatattcaggagaaccaaagcccaagactaaaatcccagaaaggcgtgtagtaacacagggaccagaggaggaatttggaatgtctttaattaaacataactcatgtgttattacaacagaaaatgggaaattcacaggtcttggagtatacgacagatttgtggtcgtaccaacacatgcagatcctggaaaggaaattcaggttgatggtataactacaaaagtcgttgactcatatgacctatacaacaagaatgggataaagctagaaataacagtacttaaattagatagaaatgaaaaatttagagatatcaggagatatatacctaacaatgaagatgattaccccaattgcaacttagcactgctagcaaaccagcctgaaccaactataatcaatgttggagatgttgtatcctatggcaatatactgctcagtggcaatcaaacggctagaatgcttaaatacagttacccaactaaatctggttactgtggaggtgtcttatacaaaattgggcaagtgcttggaatacatgttgggggcaatggtagggatggtttctcagctatgttactcagatcctatttcactgatgttcagggccaaataacgttatcaaagaagaccagtgaatgtaacctacccagtatacacaccccatgcaaaaccaaattgcagcctagtgttttctatgatgtattccctggttcaaaagaaccagctgtgttgtctgaaaaagatgcccggttacaagttgatttcaatgaagcactattttctaaatacaaagggaatacagattgctccattaatgaccacataagaattgcatcatcacattatgcagcacaactcattaccttagatattgacccaaaacctattacacttgaggacagtgtctttggcactgatggattagaggctcttgatttgaacactagcgcaggatttccatatattgcaatgggagttaaaaagagagatttaataaacaacaagaccaaggatataagcaaacttaaagaagcaattgacaaatacggagttgacttacctatggtcaccttcttgaaagatgaactcagaaagcatgaaaaggtaattaaaggtaaaactagagttattgaagctagtagtgtgaatgataccctattgtttagaacaacttttggcaacctcttttcaaagttccacttgaatcctggaattgttactggatcagcagttggatgtgatccagaggtgttttggtcaaaaataccagcaatgttggatgataaatgtattatggcttttgattatacaaattatgatggtagtatacaccctatttggtttgaagctcttaaacaggtactggtagatctatcatttaatccaacattaatagatagactatgcaagtctaaacacatcttcaaaaatacatactatgaagtggagggaggtgtaccatctgggtgttcaggtactagtatttttaacactatgatcaataatattatcataaggaccttagtgttagatgcatacaagaatatagatctagataagcttaagataattgcctatggtgatgatgtcatattctcatacatacatgaactggacatggaggctatagcaatagagggtgttaaatatggtttgactataactcctgctgataaatctaacacatttgtaaaattagactatagcaatgttacttttttaaaaagagggtttaagcaagatgagaagtataactttctaatacatccaactttccctgaagatgaaatatttgaatccatcagatggacaaagaaaccatcacaaatgcatgaacatgtgttgtctctgtgtcacttaatgtggcacaatggacgtgacgcatacaaaaaatttgtggagaagatacgcagtgtaagcgctggtcgtgcactgtacatccctccgtatgatttgcttttgcatgagtggtatgaaaaattttaaagatatagaaatagtaaactgatagtttattagttttat

SEQ ID NO: 2 - protein, 2150 aa, synthetic construct
MGAQVSRQNVGTHSTQNSVSNGSSLNYFNINYFKDAASNGASKMGFTQDPSKFTDPVKDVLEKGIPTLQSPTVEACGYSDRIIQITRGDSTITSQDVANAIVAYGVWPHYLSSKDASAIDKPSQPDTSSNRFYTLRSVTWSSSSKGWWWKLPDALKDMGIFGENMFYHYLGRSGYTIHVQCNASKFHQGTLIVALIPEHQIASALHGNVNVGYNYTHPGETGREVKAETRLNPDLQPTEEYWLNFDGTLLGNITIFPHQFINLRSNNSATIIAPYVNAVPMDSMRSHNNWSLVIIPICPLETSSAINTIPITISISPMCAEFSGARAKRQGLPVFITPGSGQFLTTDDFQSPCALPWYHPTKEISIPGEVKNLVEICQVDSLVPINNTDTYINSENMYSVVLQSSINAPDKIFSIRTDVASQPLATTLIGEISSYFTHWTGSLRFSFMFCGTANTTVKLLLAYTPPGIAEPTTRKDAMLGTHVIWDVGLQSTISMVVPWISASHYRNTSPGRSTSGYITCWYQTRLVIPPQTPPTARLLCFVSGCKDFCLRMARDTNLHLQSGAIAQNPVENYIDEVLNEVLVVPNINSSNPTTSNSAPALDAAETGHTSSVQPEDVIETRYVQTSHTRDEMSLENFLGRSGCIHESKLEVTLANYNKENFTVWAINLQEMAQIRRKFELFTYTRFDSEITLVPCISALSQNIGHITMQYMYVPPGAPVPNSRDDYAWQSGTNASVFWQHGQAYPRFSLPFLSVASAYYMFYDGYDEQDQNYGTANTNNMGSLCSRIVTEKHIHKVHIMTRIYHKAKHVKAWCPRPPRALEYTRAHRTNFKIEDRSIQTAIVTRPIITTAGPSDMYVHVGNLIYRNLHLFNSEMHESILVSYSSDLIIYRTNTVGDDYIPSCDCTQATYYCKHKNRYFPITVTSHDWYEIQESEYYPKHIQYNLLIGEGPCEPGDCGGKLLCKHGVIGIVTAGGDNHVAFIDLRHFHCAEEQGVTDYIHMLGEAFGNGFVDSVKEHIHAINPVGNISKKIIKWMLRIISAMVIIIRNSSDPQTILATLTLIGCSGSPWRFLKEKFCKWTQLNYIHKESDSWLKKFTEACNAARGLEWIGNKISKFIEWMKSMLPQAQLKVKYLNELKKLNLYEKQVESLRVADMKTQEKIKMEIDTLHDLSRKFLPLYASEAKRIKTLYIKCDNIIKQKKRCEPVAIVIHGPPGAGKSITTNFLAKMITNDSDIYSLPPDPKYFDGYDQQSVVIMDDIMQNPAGDDMTLFCQMVSSVTFIPPMADLPDKGKAFDSRFVLCSTNHSLLTPPTITSLPAMNRRFFLDLDIIVHDNFKDPQGKLNVAAAFRPCDVDNRIGNARCCPFVCGKAVSFKDRNSCNKYTLAQVYNIMIEEDRRRRQVVDVMTAIFQGPIDMRNPPPPAITDLLQSVRTPEVIKYCEGNRWIIPAECKIEKELNLANTIITIIANVISMTGIIYVIYKLFCTLQGPYSGEPKPKTKIPERRVVTQGPEEEFGMSLIKHNSCVITTENGKFTGLGVYDRFVVVPTHADPGKEIQVDGITTKVVDSYDLYNKNGIKLEITVLKLDRNEKFRDIRRYIPNNEDDYPNCNLALLANQPEPTIINVGDVVSYGNILLSGNQTARMLKYSYPTKSGYCGGVLYKIGQVLGIHVGGNGRDGFSAMLLRSYFTDVQGQITLSKKTSECNLPSIHTPCKTKLQPSVFYDVFPGSKEPAVLSEKDARLQVDFNEALFSKYKGNTDCSINDHIRIASSHYAAQLITLDIDPKPITLEDSVFGTDGLEALDLNTSAGFPYIAMGVKKRDLINNKTKDISKLKEAIDKYGVDLPMVTFLKDELRKHEKVIKGKTRVIEASSVNDTLLFRTTFGNLFSKFHLNPGIVTGSAVGCDPEVFWSKIPAMLDDKCIMAFDYTNYDGSIHPIWFEALKQVLVDLSFNPTLIDRLCKSKHIFKNTYYEVEGGVPSGCSGTSIFNTMINNIIIRTLVLDAYKNIDLDKLKIIAYGDDVIFSYIHELDMEAIAIEGVKYGLTITPADKSNTFVKLDYSNVTFLKRGFKQDEKYNFLIHPTFPEDEIFESIRWTKKPSQMHEHVLSLCHLMWHNGRDAYKKFVEKIRSVSAGRALYIPPYDLLLHEWYEKF

SEQ ID NO: 3 - DNA, 7114 bp, synthetic construct
  source 1..7114: mol_type = other DNA
ttaaaactgggtcgtggttgttcccaccacgactatctacggatgtagtgctcttgtattccggtacgcttgcacgccagttttgtcacccccccctacattgtaacttagaagattaacacaaagaccaataggcggcaatgaaccacattgtcaacggtcaagcacttctgtttccccggtcaatctagatatgctttaccaaaggcaaaaactggagcgatcgttatccgcaaggtgcctacgggaaacccagtagcattctttatttgatctggttggttgctcagccgttaaacccaaacggtaaacctggcagatgaggctggaagatccccaccagcgatggtgttccagcctgcgtggctgcctgcacacccgaaagggtgtgaagccttttcaaagacagggtgcgaagagtctactgtgctcaccttgattcctccggcccctgaatgtggctaatcctaaccccgtagctgtggtgtgcaatccagcacattcgcagtcgtaatgggtaactgcgggatggaaccaactactttgggtgtccgtgtttctctttttccttttatatatgcttatggtgacaattatacggatatagttgtcatcatgggcgctcaagtctcaaggcagaatgttggtacccactctacacaaaacacagttacaggaggatccagtcttaattattttaatattaattacttcaaagatgctgcttcatctggagcgtcaaggttggacttttctcaagacccaagtaaatttacagacccagtcaaagatgttttaaccaagggtatcccaaccctacagtcacctacagtagaagcttgtgggtattcagatagaataatacaaattaccagaggagacacaacaataacatcccaggacattgcaaacgctgtagttggatatggagtttggcctacttatttagattcaaaggatgcctcagctatagataagcccacacaaccagatacttctgcaaataggttttacacactggaaagtaaggaatggactccagatagtaaaggatggtggtggaaattaccagatgcactcaaagatatgggtgtttttggtgaaaacatgttctaccatgcacttggtaggtctggttacctaattcatgttcagtgcaatgcaagtaaatttcattcagggacccttcttgttgttgctatcccagagcaccagttggcatatataggcacaggaaatgtaacagtaggatacaaacacacccatcctggagagactggaagagtcatagcaacaaccacagacaaacagacaagacagccatcgtttgacagttggctcaattgtaatggtacccttttaggcaatgccttaatatttccacatcaatttatcaatttgagaacaaacaatgctgccaccttgatcctgccgtacgtaaatgcaactcccatggattccatgttaagacataataattggtcattactaatagtgcctgtatcagaactaagaggtgacaccagtataccaattactgtatcaatctctcccatggcagctgaattttctggagcacggaaccgcagtgcacgtgtggaaggtttaccagtgatgttaacaccaggatcaggacaattcttgacaactgatgacatgcaatctccatcagttttaccttacttccactcaacacaagaaatcttcataccaggagaagttaaaaatcttattgaattgtgccaggtggacaccatggtaccattaaacaacttgcacataaataaaaacaagattggaatgtatgctctaccacttacacgacagaatacaccagctgctgaactctttgcaatgccagtcgacattacatcttcacctttagcaaccacgcttttaggagagattgcttcttattacaccaactggacaggtagtataagattgagtttcatgttttgtggcagtgcaaacacctcactcaagcttcttctagcatatactccacctggagtgagtaaaccaaccagtaggagagaagctatgctgggcacccatcttgtatgggatgtaggtcttcaatccacatgttctttagtcatcccgtggatatcagcatcacattttcggaataccacgccagacacctactcaaaagctggttatgtgacatgctggtatcagacaaatttcatcacagcaccaaacacaccacctacagctgacattatttgtctggtttcagcatgtaaggatttttgcttacgcatggctagagatactaaccagcacactcagcttggagcaatagagcaaaaccctgttgaacaatttgcagaagcagtccttgatcaagtattagtagttccaaacactcgacccagcgatgggttgattgcaaactcagccccagctttggatgcagctgaaactggacacaccagttcagtgcagcctgaggaccttatagagactagatatgtgattgcagaccaaaccagacatgaaacctccattgaatcttttttgggtagggctggatgtgtggccactattagtttagacattaaccatgatgactaccaaaagaattacaaaaattgggcaattagtttacaagaaatgtcacaaattaggaggaaatttgaaatgtttacatatgtcagatttgattccgaaataacaatagtaccatgtgttgctgccacagaaggtaacttgggacacattgttgtgcaatacatgtttgtaccaccaggagcacctctccctgttagtagaactgacaacacttggcaatctagcacaaatgcatcagtcttttggcaggttggtcaaacttatcccagattttctatacctttctcaagtatagcttcagcttactacatgttttatgatggatacgacactgatggcacagatgcagtgtatggtgttagtgtgactaaccatatggggactatatgtgttagaattgttacagaccaacaacaacatagagttaagatcgactccatggtatatctaaaagctaaacacatcaaggcatggtgtcccagacctccaagagcagtcacatataaccatacatataatccaaattatgttagggctgatgaaacagccacaaaagtccaaactagagcaaatgtcacaacagtaggtccatcagacatgtttattcacgcctcagagtttttatacagaaattaccatctcactccagaaaaggaactgaaagaagcatgccaaattgtacatactgcagatctagttatacaccgcacaagagacaaaggtgatgactatatcccacaatgcaattgtacagattgctgttactactgtgcccacaaagacaggtatattcccatcaaagtagaataccatagctactacaccatccagaaatcagattattatccaaaacatatacagtatgatatactaattggtgagggaccttcgcaaccaggtgattgtggaggaaaacttttatgcagacatggtgttattggtatggtgacagctggaggggaaggacatgtagcatttactgatttgaggaagtatagaatggtagaggctgaggagcaaggtataacagattatgttaaatccttaggtaatgcttttggagtaggatttgtagatcaaattaaagaacaaattaataatataaacccattaaataaaatcagtgctaaagtgatcaaatggctaatcagagtaatatcagcactagtgatagcagtgcgtagccaaggggatccagcaacactatcagctactctacttttacttgggtgctctgattccccgtggcggttcctgaaacagaaggtgtgtacatggttggggcttagatacatacacaaagaatcagatggttggatcaagaaatttactgaaatgtgcaatgcagccagaggcttagaatggatagggtgtaagatttcaaaattcattgactggctgaaatccatgctacctcaagcacagagcaaaatcaagttcctccacttcatgaaacaattgcagctaaaagaaaaacagatcgatggtttaccttatgcaacagttaagcaacaagaagattatctcaaagaaatggaagagatgttggacatttcaaataaactattaccactatacccaaaggagaataagattataaaagatctactcaagcaagctaaaagcatgacaacaacatcaaagagagttgaaccagttgcaatcatgtttcatggagatccagggtcagggaaatcagtgtgcacaaacatccttgcccgcatgataactaatccatcagacatatattccctacccccaaatccaaagtattttgatggataccatcaacagactgtagtaataatggatgatgtgatgcaaaacccagatggggaagacatgagcaccttctgccaaatggtttcctcagttaattatgtagtgccaatggctgatctaccagataagggtaccctgttttcatcagattatgtcttttgcagtacaaatcagcatgttttgattcctccaaccatatctaccatacccgccctgaatcggcgattgttcttcgatctaactgttaaggtgaaccccagatatcaggaagcaggaaaactaaatctggactgtgcattaagaccatgcaatcatgagttaaaggttggaaacgcaaggtgttgtcctctcatttgtggtaaggctataaccttcattaacagacacaacaatgaagaattgacactgtcccaaatatacaatcaagtagtaaatgaacacaaccgcagattaaacgtgtcaaaacacatggaagcaattttccagggacctattgatatgcaagcacctcccccaccagcaatagttgatttacttagatccaccagaaatgaggatgtcatcaactactgcaaaaaccaaaattggactattcctgctgacatcagtattgagagagaattgaatctggtaaatctttcaatatcaattctggcaaatctaattagtgtgataggaatcatttacattatatataaattatttgtatcattacaaggaccttatagtggactacccaccaagaaaaaagtgattccagagaaaagagtggtagtacaaggtccttctactgaatttggcctaagtttaataaaacataacacatgtattgtagaaacagaaaatggtaagtttacaggtttaggaatatacgataacctattagtcatccccacacatgctgatcccagtagaactgtcaaggtggatggagtggaaacagaagtgttggactcttacgatttgtacaacaaagaaggagtaaaattagaaatcacagttctgatcttatccagaaatgaaaagtttagggatattagaaaatacataccaaattctgaggatgattatgtcaactgtaatctggctttagttgcaaaccaagatatgccacagatactggaggtcggagatgttgtctcttatggcaatatactcttgagtggtaataacaccgccagaatgctcaaatatgaataccctacaaaatcgggtttttgtggaggtgttttgtacaaggtgggtcaagtaattggcatacacgtaggtggcaatggaagacaaggattctcagccatgttacttaaaagatatttcaaccaacaacaaggtcagataattctaaagaaaccagtcaaggaagttgattatcccagtatacatacccctactaagacaaaactccaaccaagtgttttccatgatatttttcccggagttaaggaacctgcagttctaacagaaaaagacccccggttggaagtagacttgaactcctctcttttttcaaaatatgctggaaatgttaatttagaaatgaatgagtacatgattgttgcagcatcccattatgcatcacaacttgaaacactagacatatccaaccagcaaatgtctattgaggaatgtgtgtatggaacagataacttagaagccttggatttaaatactagtgcaggatttccctatgtggctttgggaattaagaagaaggacttaataaatagagagactagagatgtcagtaaaattaaaaattgcttagacacttatggtgtagatttaccaatgataacatatttaaaagatgagctcagaacaccagaaaaaataaagttaggaaaaactagggtaatagaagcaagtagtttaaatgatactattcatatgagaatgctctttggaaacttgtttaaagcctttcacgcaaatccaggcatagtaacagggtgtgcagttggttgtgatccagaaacattttggtctaaaattcctccaatgctgggtgatgggtgtgtgatggcatttgattatacaaattatgatggaagtttacatccaatatggtttaggctacttgaaagagtgctggataggttgggattcccaggctgtgcagttagaaaattatcacactccacccatatatacaaaggaatgtactatgaagttgatggaggaatgccatctgggtgtgcaggtacttcaatcttcaactcaatgatcaataacatcataatcagaacaataattctacatgcttacaagaatatagacctggatcagttgaggatacttacatatggtgatgatataatttttacttacccagataaactagacatggcttatttagcccaaattggagaaaaatatggtttaaaaatgacacctgcagacaagtcagatacatttaaggatttggatctgagtacagcaactttcctaaaaagaggctttaaacctgactcaaaacacccatttttagtccatcccatatatccaatccaggatatctatgaatcaatcagatggacaaagaatcccagatgtttacaggagcatgtgctcagtttggctcacttatgctggcatagtggaccagagcagtatgctgattttgtcaagagaatcagatcaacatctgttgggaagaacctatacataccatcttatgatgtactactttatgagtggtatgagaaattttaagttaatatatacagttactatttaggtagtttgggtgtat

SEQ ID NO: 4 - protein, 2165 aa, synthetic construct
MGAQVSRQNVGTHSTQNTVTGGSSLNYFNINYFKDAASSGASRLDFSQDPSKFTDPVKDVLTKGIPTLQSPTVEACGYSDRIIQITRGDTTITSQDIANAVVGYGVWPTYLDSKDASAIDKPTQPDTSANRFYTLESKEWTPDSKGWWWKLPDALKDMGVFGENMFYHALGRSGYLIHVQCNASKFHSGTLLVVAIPEHQLAYIGTGNVTVGYKHTHPGETGRVIATTTDKQTRQPSFDSWLNCNGTLLGNALIFPHQFINLRTNNAATLILPYVNATPMDSMLRHNNWSLLIVPVSELRGDTSIPITVSISPMAAEFSGARNRSARVEGLPVMLTPGSGQFLTTDDMQSPSVLPYFHSTQEIFIPGEVKNLIELCQVDTMVPLNNLHINKNKIGMYALPLTRQNTPAAELFAMPVDITSSPLATTLLGEIASYYTNWTGSIRLSFMFCGSANTSLKLLLAYTPPGVSKPTSRREAMLGTHLVWDVGLQSTCSLVIPWISASHFRNTTPDTYSKAGYVTCWYQTNFITAPNTPPTADIICLVSACKDFCLRMARDTNQHTQLGAIEQNPVEQFAEAVLDQVLVVPNTRPSDGLIANSAPALDAAETGHTSSVQPEDLIETRYVIADQTRHETSIESFLGRAGCVATISLDINHDDYQKNYKNWAISLQEMSQIRRKFEMFTYVRFDSEITIVPCVAATEGNLGHIVVQYMFVPPGAPLPVSRTDNTWQSSTNASVFWQVGQTYPRFSIPFSSIASAYYMFYDGYDTDGTDAVYGVSVTNHMGTICVRIVTDQQQHRVKIDSMVYLKAKHIKAWCPRPPRAVTYNHTYNPNYVRADETATKVQTRANVTTVGPSDMFIHASEFLYRNYHLTPEKELKEACQIVHTADLVIHRTRDKGDDYIPQCNCTDCCYYCAHKDRYIPIKVEYHSYYTIQKSDYYPKHIQYDILIGEGPSQPGDCGGKLLCRHGVIGMVTAGGEGHVAFTDLRKYRMVEAEEQGITDYVKSLGNAFGVGFVDQIKEQINNINPLNKISAKVIKWLIRVISALVIAVRSQGDPATLSATLLLLGCSDSPWRFLKQKVCTWLGLRYIHKESDGWIKKFTEMCNAARGLEWIGCKISKFIDWLKSMLPQAQSKIKFLHFMKQLQLKEKQIDGLPYATVKQQEDYLKEMEEMLDISNKLLPLYPKENKIIKDLLKQAKSMTTTSKRVEPVAIMFHGDPGSGKSVCTNILARMITNPSDIYSLPPNPKYFDGYHQQTVVIMDDVMQNPDGEDMSTFCQMVSSVNYVVPMADLPDKGTLFSSDYVFCSTNQHVLIPPTISTIPALNRRLFFDLTVKVNPRYQEAGKLNLDCALRPCNHELKVGNARCCPLICGKAITFINRHNNEELTLSQIYNQVVNEHNRRLNVSKHMEAIFQGPIDMQAPPPPAIVDLLRSTRNEDVINYCKNQNWTIPADISIERELNLVNLSISILANLISVIGIIYIIYKLFVSLQGPYSGLPTKKKVIPEKRVVVQGPSTEFGLSLIKHNTCIVETENGKFTGLGIYDNLLVIPTHADPSRTVKVDGVETEVLDSYDLYNKEGVKLEITVLILSRNEKFRDIRKYIPNSEDDYVNCNLALVANQDMPQILEVGDVVSYGNILLSGNNTARMLKYEYPTKSGFCGGVLYKVGQVIGIHVGGNGRQGFSAMLLKRYFNQQQGQIILKKPVKEVDYPSIHTPTKTKLQPSVFHDIFPGVKEPAVLTEKDPRLEVDLNSSLFSKYAGNVNLEMNEYMIVAASHYASQLETLDISNQQMSIEECVYGTDNLEALDLNTSAGFPYVALGIKKKDLINRETRDVSKIKNCLDTYGVDLPMITYLKDELRTPEKIKLGKTRVIEASSLNDTIHMRMLFGNLFKAFHANPGIVTGCAVGCDPETFWSKIPPMLGDGCVMAFDYTNYDGSLHPIWFRLLERVLDRLGFPGCAVRKLSHSTHIYKGMYYEVDGGMPSGCAGTSIFNSMINNIIIRTIILHAYKNIDLDQLRILTYGDDIIFTYPDKLDMAYLAQIGEKYGLKMTPADKSDTFKDLDLSTATFLKRGFKPDSKHPFLVHPIYPIQDIYESIRWTKNPRCLQEHVLSLAHLCWHSGPEQYADFVKRIRSTSVGKNLYIPSYDVLLYEWYEKFVNIYSYYLGSLGV

SEQ ID NO: 5 - DNA, 7206 bp, synthetic construct
  source 1..7206: mol_type = other DNA
taatacgactcactatagttaaaactgggtcgtggttgttcccaccacgactatctacggatgtagtgctcttgtattccggtacgcttgcacgccagttttgtcacccccccctacattgtaacttagaagattaacacaaagaccaataggcggcaatgaaccacattgtcaacggtcaagcacttctgtttccccggtcaatctagatatgctttaccaaaggcaaaaactggagcgatcgttatccgcaaggtgcctacgggaaacccagtagcattctttatttgatctggttggttgctcagccgttaaacccaaacggtaaacctggcagatgaggctggaagatccccaccagcgatggtgttccagcctgcgtggctgcctgcacacccgaaagggtgtgaagccttttcaaagacagggtgcgaagagtctactgtgctcaccttgattcctccggcccctgaatgtggctaatcctaaccccgtagctgtggtgtgcaatccagcacattcgcagtcgtaatgggtaactgcgggatggaaccaactactttgggtgtccgtgtttctctttttccttttatatatgcttatggtgacaattatacggatatagttgtcatcatgggcgctcaagtctcaaggcagaatgttggtacccactctacacaaaacacagttacaggaggatccagtcttaattattttaatattaattacttcaaagatgctgcttcatctggagcgtcaaggttggacttttctcaagacccaagtaaatttacagacccagtcaaagatgttttaaccaagggtatcccaaccctacagtcacctacagtagaagcttgtgggtattcagatagaataatacaaattaccagaggagacacaacaataacatcccaggacattgcaaacgctgtagttggatatggagtttggcctacttatttagattcaaaggatgcctcagctatagataagcccacacaaccagatacttctgcaaataggttttacacactggaaagtaaggaatggactccagatagtaaaggatggtggtggaaattaccagatgcactcaaagatatgggtgtttttggtgaaaacatgttctaccatgcacttggtaggtctggttacctaattcatgttcagtgcaatgcaagtaaatttcattcagggacccttcttgttgttgctatcccagagcaccagttggcatatataggcacaggaaatgtaacagtaggatacaaacacacccatcctggagagactggaagagtcatagcaacaaccacagacaaacagacaagacagccatcgtttgacagttggctcaattgtaatggtacccttttaggcaatgccttaatatttccacatcaatttatcaatttgagaacaaacaatgctgccaccttgatcctgccgtacgtaaatgcaactcccatggattccatgttaagacataataattggtcattactaatagtgcctgtatcagaactaagaggtgacaccagtataccaattactgtatcaatctctcccatggcagctgaattttctggagcacggaaccgcagtgcacgtgtggaaggtttaccagtgatgttaacaccaggatcaggacaattcttgacaactgatgacatgcaatcaccatcagttttaccttacttccactcaacacaagaaatcttcataccaggagaagttaaaaatcttattgaattgtgccaagtggacaccatggtaccattaaacaacttgcacataaataaaaacaagattggaatgtatgctctaccacttacacgacagaatacaccagctgctgaactctttgcaatgccagtcgacattacatcttcacctttagcaaccacgcttttaggagagattgcttcttattacaccaactggacaggtagtataagattgagtttcatgttttgtggcagtgcaaacacctcactcaagcttcttctagcatatactccacctggagtgagtaaaccaaccagtaggagagaagctatgctgggcacccatcttgtatgggatgtaggtcttcaatccacatgttctttagtcatcccgtggatatcagcatcacattttcggaataccacgccagacacctactcaaaagctggttatgtgacatgctggtatcagacaaatttcatcacagcaccaaacacaccacctacagctgacattatttgtctggtttcagcatgtaaggatttttgcttacgcatggctagagatactaaccagcacactcagcttggagcaatagagcaaaaccctgttgaacaatttgcagaagcagtccttgatcaagtattagtagttccaaacactcgacccagcgatgggttgattgcaaactcagccccagctttggatgcagctgaaactggacacaccagttcagtgcagcctgaggaccttatagagactagatatgtgattgcagaccaaaccagacatgaaacctccattgaatcttttttgggtagggctggatgtgtggccactattagtttagacattaaccatgatgactaccaaaagaattacaaaaattgggcaattagtttacaagaaatgtcacaaattaggaggaaatttgaaatgtttacatatgtcagatttgattccgaaataacaatagtaccatgtgttgctgccacagaaggtaacttgggacacattgttgtgcaatacatgtttgtaccaccaggagcacctctccctgttagtagaactgacaacacttggcaatctagcacaaatgcatcagtcttttggcaggttggtcaaacttatcccagattttctatacctttctcaagtatagcttcagcttactacatgttttatgatggatacgacactgatggcacagatgcagtgtatggtgttagtgtgactaacgatatggggactatatgtgttagaattgttacagaccaacaacaacatagagttaagatcgactccatggtatatctaaaagctaaacacatcaaggcatggtgtcccagacctccaagagcagtcacatataaccatacatataatccaaattatgttagggctgatgaaacagccacaaaagtccaaactagagcaaatgtcacaacagtaggtccatcagacatgtttattcacgcctcagagtttttatacagaaattaccatctcactccagaaaaggaactgaaagaagcatgccaaattgtatatactgcagatctagttatacaccgcacaagagacaaaggtgatgactatatcccacaatgcaattgtacagattgctgttactactgtgcccacaaagacaggtatattcccatcaaagtagaataccatagctactacaccatccagaaatcagattattatccaaaacatatacagtatgatatactaattggtgagggaccttcgcaaccaggtgattgtggaggaaaacttttatgcagacatggtgttattggtatggtgacagctggaggggaaggacatgtagcatttactgatttgaggaagtatagaatggtagaggctgaggagcaaggtataacagattatgttaaatccttaggtgatgcttttggagtaggatttgtagatcaaattaaagaacaaattaataatataaacccattaaataaaatcagtgctaaagtgatcaaatggctaatcagagtaatatcagcactagtgatagcagtgcgtagccaaggggatccagcaacactatcagctactctacttttacttgggtgctctaattccccgtggcggttcctgaaacagaaggtgtgtacatggttggggcttagatacatacacaaagaatcagatggttggatcaagaaatttactgaaatgtgcaatgcagccagaggcttagaatggatagggtgtaagatttcaaaattcattgactggctgaaatccatgctacctcaagcacagaacaaaatcaagtttctccacttcatgaaacaattgcagctaaaagaaaaacagatcgatggtttaccttatgcaacagttaagcaacaagaagattatctcaaagaaatggaagagatgttggacatttcaaataaactattaccactatacccaaaggagaataagattataaaagatctactcaaacaagctaaaagcatgacaacaacatcaaagagagttgaaccagttgcaatcatgtttcatggagatccagggtcagggaaatcagtgtgcacaaacatccttgcccgcatgataactaatccatcagacatatattccctacccccaaatccaaagtattttgatggataccatcaacagactgtagtaataatggatgatgtgatgcaaaacccagatggggaagacatgagcaccttctgccaaatggtttcctcagttaattatgtagtgccaatggctgatctaccagataagggtaccctgttttcatcagattatgtcttttgcagtacaaatcagcatgttttgattcctccaaccatatctaccatacccgccctgaatcggcgattgttcttcgatctaactgttaaggtgaaccccagatatcaggaagcaggaaaactaaatctggactgtgcattaaaaccatgcaatcatgagttaaaggttggaaatgcaaggtgttgtcctctcatttgtggtaaggctataaccttcattaacagacacaacaatgaagaattgacactgtcccaaatatacaatcaagtagtaaatgaacacaaccgcagattaaacgtgtcaaaacacatggaagcaattttccagggacctattgatatgcaagcacctcccccaccagcaatagttgatttacttagatccaccagaaatgaagatgtcatcaactactgcaaaaaccaaaattggattattcctgctgacatcagtattgagagagaattgaatctggtaaatctttcaatatcaattctggcaaatctaattagtgtgataggaatcatttacattatatataaattatttgtatcattacaaggaccttatagtggactacccaccaagaaaaaagtgattccagagaaaagagtggtagtacaaggtccttctactgaatttggcctaagtttaataaaacataacacatgtattgtagaaacagaaaatggtaagtttacaggtttaggaatatacgataacctattagtcatccccacacatgctgatcccagtagaactgtcaaggtggatggagtggaaacagaagtgttggactcttacgatttgtacaacaaagaaggagtaaaattagaaatcacagttctgatcttatccagaaatgaaaagtttagggatattagaaaatacataccaaattctgaggatgattatgtcaactgtaatctggctttagttgcaaaccaagatatgccacagatactggaggtcggagatgttgtctcttatggcaatatactcttgagtggtaataacaccgccagaatgctcaaatatgaataccctacaaaatcgggtttttgtggaggtgttttgtacaaggtgggtcaagtaattggcatacacgtaggtggcaatggaagacaaggattctcagccatgttacttaaaagatatttcaaccaacaacaaggtcagataattctaaagaaaccagtcaaggaagttgattatcccagtatacatacccctactaagacaaaactccaaccaagtgttttccatgatatttttcccggagttaaggaacctgcagttctaacagaaaaagacccccggttggaagtagacttgaactcctctcttttttcaaaatatgctggaaatgttaatttagaaatgaatgagtacatgattgttgcagcatcccattatgcatcacaacttgaaacactagacatatccaaccagcaaatgtctattgaggaatgtgtgtatggaacagataacttagaagccttggatttaaatactagtgcaggatttccctatgtggctttgggaattaagaagaaggacttaataaatagagagactagagatgtcagtaaaattaaaaattgcttagacacttatggtgtagatttaccaatgataacatatttaaaagatgagctcagaacaccagaaaaaataaagttaggaaaaactagggtaatagaagcaagtagtttaaatgatactattcatatgagaatgctctttggaaacttgtttaaagcctttcacgcaaatccaggcatagtaacagggtgtgcagttggttgtgatccagaaacattttggtctaaaattcctccaatgctgggtgatgggtgtgtgatggcatttgattatacaaattatgatggaagtttacatccaatatggtttaggctacttgaaagagtgctggataggttgggattcccaggctgtgcagttagaaaattatcacactccacccatatatacaaaggaatgtactatgaagttgatggaggaatgccatctgggtgtgcaggtacttcaatcttcaactcaatgatcaataacatcataatcagaacaataattctacatgcttacaagaatatagacctggatcagttgaggatacttacatatggtgatgatataatttttacttacccagataaactagacatggcttatttagcccaaattggagaaaaatatggtttaaaaatgacacctgcagacaagtcagatacatttaaggatttggatctgagtacagcaactttcctaaaaagaggctttaaacctgactcaaaacacccatttttagtccatcccatatatccaatccaggatatctatgaatcaatcagatggacaaagaatcccagatgtttacaggagcatgtgctcagtttggctcacttatgctggcatagtggaccagagcagtatgctgattttgtcaagagaatcagatcaacatctgttgggaagaacctatacataccatcttatgatgtactactttatgagtggtatgagaaattttaagttaatatatacagttactatttaggtagtttgggtgtataaaaaaaaaaaaaaaaaaaaaaaaaaaaaaaaaaaaaaaaaaaaaaaaaagaagagctttagtgagggttaatt

SEQ ID NO: 6 - DNA, 7087 bp, synthetic construct
  source 1..7087: mol_type = other DNA
gcggccgcatttaggtgacactatagccaaagtagttggtcccgtcccgcatgcaacttagaagctttgcacaaagaccaatagccagtaatcatccagattacttaaggtcaagcacttctgtttccccggtcaaagttgatatgctccaccagggcaaaaacaactgcgatcgttatccgcaaggcgcctacacaaagcttagtagcattctgaaagttgtctggttggtcgttccaccattccccctggtagacctggcagatgaggctagaaactccccactggcgacagtgttctagcctgcgtggctgcctgcacaccctctgggtgtgaagccatacgatggacagggtgtgaagagccccgtgtgctcgctttgacgtcctccggcccctgaatgtggctaaccttaaccctgcagctagagcacacaatccagtgtgtttctagtcgtaatgagcaattgcgggacgggaccaactactttgggtgtccgtgtttccttgttctctttgaatatctgcttatggtgacaatatatacgtatatattgacaccatgggcgcacaagtatccaggcaaaatgttggaactcactccacacagaattctgtatcaaatggatctagtttgaattattttaatatcaattacttcaaggatgctgcttcaaatggagcatctaaattggagttttcacaagatcctagtaaatttactgatcctgttaaagatgttttggaaaaaggaataccaacactgcagtcgcctacagtggaagcttgtgggtactctgatagaattatacaaattactagaggagattcaacaataacttcacaagatgtggctaatgctgttgttgcatacggtgtttggccgcactacttgtcatctgaagacgccactgcaattgacaaaccctcccgtcctgacacatcgtccaacagattctacacactaaagagtataacttggtctaggtcttcaaagggttggtggtggaaactaccagatgcattgagagatatgggtatctttggtgaaaacatgttctaccattatctgggtaggagtgggtatacagtgcatgtgcagtgtaatgctactaaatttcatcaaggcacactggtggttgcaatgatacctgaacatcaaatcgctagtcttcaacacggcgatgtaaatgtcgggtacaattttacccacccgggagaggaagggagagaggtcaaaaacactacacgtgaacatcttcagcagcagcctactgaagaacactggctcaattttgatggtacattattaggaaatattacaatatttcctcatcaatttattaatctgaggagtaacaattctgccacaataattgtcccgtacgttaatgcagttccaatggattcaatgcgaagccacaataattggagtttagtgataatcccaatttgccctcttgagacaatgagcaccatcaccacggtacctattacaatttccattagtccaatgtgtgcagagttctctggtgcacgtgctagaagccaaggcttaccggtgttaattacaccaggctcaggtcaatttctaacaacagatgactttcaatctccatgtgcacttccttggtaccatccaactaaagaaattttcattccaggtgaagtcaaaaacttagttgaaatctgtcaagtagatagcttgataccaataaacaaccttgaagggaacataactagtgaagctatgtattcaatagaactgcagtcatcgactgaacaaagtaagatattttctattagaacagatattgcttcccaacctttagctactactctaattggtgaaatttctagttattttacacactggacagggagtttacgtttcagttttatgttctgtggtactgccaatactactgtcaaacttttattagcatacacaccacctggtattgcagaacctacaacaaggaaaaatgcaatgttgggtacccatataatatgggacataggtttgcaatctacaatatcaatggtagtaccttggatcagtgccagccattatagaaacacatcaccagatgtgtctacatctggattcataacttgctggtatcaaactggattagttattccacctgacactccttccacagctagattattgtgctttgtctctgggtgcaaagatttctgcttgcgcatggcccgagacaccaatttgcacatgcagaaaggtataatagcacaaaatccaattgagaactatgtagatgaagttcttaatgaagtcttagttgttcccaatatcaatagtagtcatcccacaacatcaaactctgctccagcattagacgctgcggaaacgggtcacactagtaatgttcaaccagaagatgtcattgaaaccaggtacgttcaaacatcacaaacaagagatgaaatgagtttagaaagtttccttggtaggtcagggtgtatacatgaatctaaattaaaagttgagatcggaaactatgatgaaaacaattttaatacttggaatattaatttacaggaaatggcccaaatcagaagaaagtttgaactgtttacttacactagatttgattctgaaattactttggttccatgcatttctgctcttagtcaagatattggtcacatcacaatgcagtatatgtatgtcccaccaggtgctccaataccgaaaagtagaaatgactatgcatggcaatctggaacaaatgcgtccattttttggcaacatggacaaacatatccaaggttctccttaccctttttgagtgtggcatctgcttattacatgttttatgatggatacaatgagaaaggcacgcattatggaacagttagcacaaacaacatgggcacattgtgctccagagtggtaacagagaaacacattcatgatatgcggataatgacaagggtctaccacaaagctaaacatgtcaaagcatggtgtccgcggccacccagagcacttgaatacacacgcgctcaccgtactaatttcaaaattgaaggtgaaaatgtcaaatcaagggttgcacatagacctgcagtgataacagctggccccagtgatatgtatgtacacgtagggaacctcatttacagaaaccttcatctctttaattctgagatgcatgaatctatcttggtgtcttactcttcagatctaatcatttaccgaacaaacactataggtgatgattatatcccttcttgtgattgcactcaagctacatattactgtaaacacaaaaatagatattttccgatcactgttacaagccatgactggtatgaaatacaggagagtgaatattaccctaagcacatacaatacaacttgttaattggtgaagggccttgtgagccaggtgattgtggtggaaaactactatgtaagcatggagttataggcattgtaacagctggtggtgataatcatgtagctttcattgaccttagacaatttcattgtgctgatgagcaaggaatcacagactacatacacatgttaggtgaggcatttggaaatggatttgtagatagtgtgaaggagcatgtgcatgccattaacccaattggtaatattagcaagaaaattgtcaaatggatgttaaggataatatcagcaatggttataataattagaaactcttctgacccacaaaccatattggcaacactaacactgattggatgttcaggatctccgtggaggtttctcaaagaaaaattttgtaagtggacacagcttagttacatacataaggaatcagattcatggttaaagaaattcactgaagcatgtaatgcagcaagaggtcttgaatggatagggaataaaatatccaaattcattgagtggatgaagtcaatgcttccgcaagctcaacttaaggtcaagtacttgaatgaacttaagaaattaagcctatatgaaaaacaagttgaaagtctaagggtggcagatataaaaacccaggagaaaatcaaaatggagattgatactttgcatgacttgtcatgcaaattcttgccactgtatgcaagtgaagcaaaaagaataaaaaccctccacattaaatgtgataatataattaaacaaaagaagagatgtgaaccagtagctatagtcattcatggaccaccaggcgctggtaagtctataacaacaaatttcttagccaaaatgatcactaatgacagtgatatttattcccttcctccagacccaaaatactttgacggttatgaccagcagagtgtggttataatggatgacatcatgcaaaatccaactggagatgacatgactttattctgtcagatggtatctagtgttacctttataccaccaatggctgatctgccagacaagggtaaagcttttgattctagatttgttttgtgtagtacaaaccattctctcttggcaccacccacaataacctcgctccctgctatgaatagaagattcttttttgatctggatataatagtacatgataatttcaaagattcacagggaaaacttaatgtggcagcagctttcaaaccatgtgatgtagatatcaaaataggaaatgcacgctgttgtccatttgtgtgtggaaaagcagtctccttcaaagatcgcaattcttgtaataagtataccttagcacaaatttataacataatgcttgaagaggatcggcgcagaagacaaatggttgacgtcatgtcagctatattccaggggccaattgacatgaaaaatccaccaccacctgccatcactgatttactccaatccgttagaacccccgaggttattaaatattgtgaagaaaacagatggataatcccagcagagtgtaaaatagaaaaagagctaaacttggctaatacaattataacaattattgctaatgttattagtatagcaggtattatatatgtgatttataaattgttctgcacactacaaggaccttactcaggggaaccaaaaatcaaaactaagattccagagaggcgtgtggtggcacaaggaccagagggggagtttggaatgtctctgatcaaacataactcgtgtgttgtcacaacagaaaatggcaaatttacaggccttgggatatatgatagaactttgattgtgccaacccatgctgatcctggaaaggaaatccaaattgatggcataacaacaaaagtgattgattcatatcacctatataacaaggatggtattaaacttgagataacagttctaaagttagataggaatgagaagtttagagatattagaaagtatataccaaacaatgaagacgactatccagattgtaacttggccctattggcaaaccaacctgaacccactataattaatgttggtgatgttgtatcctatggtaacattttgcttagtggaagccagactgccagaatgctcaagtacaattatccaactaaatctggatattgtgggggtgtactatacaaaattgggcaggtattagggatacatgtggggggaaatggcagagatggcttttcagctatgttactcagatcttattttgctgacacccaaggccaaataaccttgtctaaaagaaccagtgagtgtaatcttcccagcatacataacccgtgtaaaaccaaattacaacctagtgtgttttatgatgtgttccctggttcaaaagaaccagctgtgctgtctgacaaggatactagattgcaggttgattttaatgaatcactattttcaaaatacaagggaaacacacattgtgtgatgaatgaacacataaaaattgcatcagcacattatgcagcacagttgattactcttgacatagatccaaatcctataacacttgaagatagtgtctttggcactgatggtctggaagctcttgatttgaatactagtgcaggttttccatatattacaatgggaattaaaaagagagaccttataaacaacaagaccaaggacataagtaagcttagagaagcccttgacaaatatggagttgacttaccaatggtcactttcctaaaagatgaacttagaaagcaagagaaaataataaaaggaaaaactagagttattgaagctagtagtgttaatgatactttgctgtttagaacgacctttggaaaccttttctcaaaatttcatttgaacccaggtattgttactggatcagcggttggctgcgatccagagactttttggtcgaaaataccagcaatgctagatgataaatgcattatggcctttgattacacaaattatgacggtagtatacatcctatttggtttgaagctcttaaacaagtactcacagatttatcattcaacccgatgttaatagacagattatgtaggtctaaacacattttcaaaaacacatattatgaggttgagggaggtgttccatctggatgctcgggcactagtatatttaacactatgataaataacatcattataagaactttagtattagatgcatataagaacatagatttagacaaacttaagataattgcctatggtgatgatgtcatattttcatacacatacgaattggatatggaagctatagcaatagaaggtgttaaatatggcctaaccataactcctgctgataaatctactactttcagaaaactagactataacaatgttactttcctaaaaagagggtttaagcaagatgaaaagtatagttttttaatccatcccacttttcctgagaatgaaatatttgaatcaattagatggacaaagaaaccttctcaaatgcacgagcatgttctgtctttgtgtcatttaatgtggcataatggacgtgatgcatacaaaggatttgtggagaagatacgcagcgttagcgctggtcgcatgctgtatatccctccatatgatctgctcttgcatgagtggtatgagaaattttaaatagatatagaaatattaaacaattagtttattagttttagaaaaaaaaaaaaaaaaaaaaaaaaagactctcgag

SEQ ID NO: 7 - DNA, 7178 bp, synthetic construct
  source 1..7178: mol_type = other DNA
gcggccgcatttaggtgacactatagataaaactggatccaggttgttcccacctggatctcctacgtggagttgtactctattattccggtaatcttgtacgccagttttatatcccctacccctttgtaacttagaagttaaacacacagaccaataggcggtggctatccaagtcactaatggtcaagcacttctgtttccccggtcaagattgatacgctccaacagggcgaaaacagtcaagatcgttaaccgcaaagtgactacgcaaagcttagtaatgccttgaagatctatggctggtcgttccgctataaccccctagtagacctggcagatgaggctagaaacaccccactggcaacagtgttctagcctgcgtggctgcctgcacaccctttcgggtgtgaagccatatatttgacaaggtgtgaagagccccgtgtgctcactttgagcctccggcccctgaatgtggctaaccttaaccctgcagctagcgtatgtaatccaacatattgctagtcgtaatgagcaattgcgggatgggaccaactactttgggtgtccgtgtttcacttttttccttttatttgcttatggtgacaatatatatagtatatatattggcatccatgggcgcacaagtgtctagacagaatgttggaactcattctactcaaaactcagtttcaaatggatcaagtctaaattactttaacataaattattttaaggatgctgcatccagtggagcttcaaaacttgaattttcacaagatcctagtaaattcactgacccagttaaagatgtcctagagaaaggaattccaacattacaatctccaactgtggaagcatgtggctattcagatagaataatacaaattacaagaggtgattccacaataacgtcacaagatgtggctaatgcagttataggttatggtgtctggccacattacttgagtgatgaggatgctactgctattgacaaacctacacaaccagacacttcatctaacagattttacactttggaaagtaaaatatggcatactgattccaagggatggtggtggaagttgccggatgcacttaaagatatgggaatatttggagaaaacatgtactatcactatcttggtaggagtgggtacacagtacatgttcagtgcaatgccagtaaattccatcaaggaacgctactggtggtgatgataccagagcaccagttagcaaatgcaaattctactaaagcaggagccgggtataattacacacatcctggtgagcggggaagacaagttggtgatcaacgcgcaaacatcactggagcacatcctagtgatgataactggctcaattttgacggcacaatgctaggtaatgtcctaatattcccacatcaatttattaatcttagaagcaataactctgcaacaataatagtcccttatgtaaatgctgttccaatggattcaatgctcagacacaataactggagcctggtaataataccaattagcaagttgcaagctgataatactgctagtgttacagttccaatcactatatcaataagcccaatgtttgctgaattttctggagcacgagcaagacctacaagagctgttacagaaggcttacctacatatacaacaccaggttcaggtcagtttatgacaactgatgattttcaatcaccttctgcattaccttggtatcatccaactaaagaaattttaattccaggagaagttaaaaatttaatagaaatgtgtcaggttgatacacttatacctatcaataacttagaaaataatttaaggaaagttaatttatacactgttgaactaaccaatcagacaacaccagcccaaaagatcttctctattaaagtggacattgcctcacaacctttagcaaatactatgcttggccaaattgctagttatttcacacactggactggtagtttacgctttagttttatgttttgtggtacagcaaacacaactcttaaattacttctagcatacacaccaccaggcattggggaaccaacagacaggaaacaagctatgcttggtacacatatagtctgggatgttggattacaatcgacagtatctttggttgtcccgtgggttagttctagtcattttagatacacctcaagagatgaacactcagcagcaggatacataacatgctggtaccaaacaagtcttgtctttcctccgagcactcctggtacagctgatatgttgtgttttgtatcagcttgtaaggacttttgtctacgtatggctagggacacagacatgcacactcaaaatgatgccattgaacaaaatccaattgaaaattatgtggatcaagtgcttaacgaagttttggttgtaccaaatattaaagagagccaccctagtacatcaaactctgccccaattttagatgctgctgaaactggacatactagtaatgtgcaaccagaggacactattgaaacccgctatgtccaaaccacacaaactagagatgagatgagcattgagagtttccttggtagatcaggatgtgtacatacctcaacaattgaaacgacactcaagcacaaagacagattcaaaacatggaatattaatcttaaagagatggctcaaattaggagaaagtttgaaatgtttacatacgtaagatttgattcagaaataaccttagttccatctattgcaggacacggtgcagatataggacacatagttatgcagtacatgtatgtaccacctggggctccactaccagaagacagagaacacttcgcctggcaatcaagcaccaatgcatcaatattttggcaacatggacaaccctttcctagattttcattacccttcttgagtgttgcatctgcttattatatgttttatgatggttataatggtgacattcctggagcaaaatatggtaccactgtggttaatcgcatgggggcactgtgtatgagaattgtcacaaacaagcaagttcatgatgttgaagttacaactaatatttatcataaagctaaacatataaaagcatggtgccctagaccacctagagctgttccatacaaatatgttaactataataattatgcagatagtagtaatgttgacatttttatagaatcaagaagaagtttactaacagctggaccaagcgacatgtatgtccatgtcggtaatctgatatatagaaaccttcatctcttcaattcagaaatgcaagattcagtgttagtgtcatactcttcagatttagtcatttaccgaacaaacacaacaggtaatgactacattcctacttgtgattgtactgatgccacatattactgtaagcataaagatagatactatcccattaaggtcaccagtcatgattggtatgaaatacaagagagcgagtactatcccaaacacatccagtacaatctcctgattggtgaaggaccatgtgagccaggtgactgtggtggaaaactattgtgcaggcatggagtaattgggatagtaactgcaggtggggagggacatgtagcttttattgatcttagacagtttcattgtgcagatgagcaaggggtgactgattatattcatatgttaggtgaggcttttggtgcaggttttgttgatagtgttaaggagcaaataaatgccattaatccaattaataatattagcaagaaaataattaaatggcttcttagaataatttcggcaatggtcataattattagaaattcctctgatccacaaacaattatagctacattgactttgattgggtgttcaggttccccttggagattcctcaaggaaaaattttgtaaatggacacagttgaattacatacataaagaatctgattcatggctcaaaaaatttactgaaatgtgtaatgcagccagaggtttggagtggattgggaataaaatatcaaaatttattgaatggatgaaatccatgcttcctcaagctcaacttaaagttaaatatctgagtgaacttaaaaaattaaactttcttgaaaagcagatagagcaccttagagttgctgattcccaaactcaggaaaaaattaagcatgagattgacactttgcatgacctttcttgtaaatttcttccactttatgctagtgaagcaaaaagaattaagatattacacaataagtgtagcacaattatcaaacaaaagaaaaggtgtgagccagttgctgttgttatacatggagagcctggtactggtaagtctatgaccacaaatttccttgccagaatgataacaagtgatagtgacatatactctttaccacctgaccccaaatattttgatggatatgaccaacaaagtgtggtcattatggatgacatcatgcaaaatccaagtggtgaagacatgaccttattttgtcagatggtgtctagtgttacattcatacccccaatggctgatttgcctgataaaggtaaaccttttgattcaagatttgtcttgtgtagtactaatcataatatgctagcaccacctacaatcacttcattgcctgctatgaataggagattctattttgacctagatattgtagtatgtgacaaatataaagattcacaaggaaagcttaatgtgtcagctgcttttcaaccatgcgatgttgacacaaaaattggaaatgcccgttgctgtccttttatttgtggtaaagctgtcatctttagggaccgcaatacctgtcaaacctacaccttggcacaagtgtataaccaaatgcttgctgaagacaaaaggcgcaggcaagttattgatgtaatggcagcaattttccaaggccctattactattgaaggaccaccacctgcagcaataaacgacttactgaaatcagttagaactccagaagtaattagatattgtgaagagaataaatggatagtgcctgctgagtgcaaagttgaaagagaccttaatatagcaaatacaattattacaattatagctaatattattagtatatctggaataatttatgtaatttacaaacttttctgcacattgcaaggaccatactcaggtgagcctaagccaaagaccaagataccagagaggagagttgtggctcagggtccagaagaagaatttggtagatcactcattaaacataattcatgtatagtgtcaacgcagaatggaaaattcacaggcttagggatttatgatagaacattaatcttaccaacacatgcagatcctggtaatgagatacagattgatggaattacaactaaagtagaggattcttatgatttgtataataaagatggaataaagctagaaattacagtcctaaaacttaagagaaatgaaaagttcaaagacattaggaaatacataccagagaatgaagatgattatccagattgtaatttggccttatctgccaatcaaccagaaacaaccataatcaatgtaggggatgtagtatcttatggcaatattttacttagtggaaatcagacagctaggatgcttaaatataattacccaacaaaatcaggttattgtggtggcatcctgtataaaattggtcaagtccttggaatacatgttggaggaaatgggagagatggatttgctgccatgctgctacgatcttacttttctgagacacaaggtcaaataattacttccaaatctacaagtgagtgtggtttacctagtatccacacaccaaacaaaacaaagttacaacctagtgttttttatgatgtgtttcctggtaataaagagccagctgttcttacaagtgatgaccctaggctagaagtagatttcaaagaagctttgttttctaaatataaaggaaataatcagtgtaccatgaatgatcatattaaagtggctatttctcattactcagcacaattgatgacattagatattgatccaaaacctatatccttagaagaaagtgtttttggcatagagggcttggaagcactagatctcaacaccagtgcaggatttccttatgtatctatgggcattaagaaaagagatttaattaataaacaaactaaagatctgacaaaattaaagatggcattagataagtacggtgttgatttgcctatggtaacctttcttaaagatgagcttaggaaaaaggagaaaatctgtgctggaaagacccgtgtaattgaagctagtagtgtaaatgatactgtgttatttagaacaacttttggcaatcttttttcaaaatttcacttaaatccaggtattatcactggctctgctgtgggatgtgatccagaaacattttggtcaaaaataccagttatgctagatggagaatgtataatggcctttgattatacaaattatgatgggagtatacatcctatttggtttcaagccttgaaagaagtcttagtcaatttatcatttgaatcaactttaatagataggttgtgcagatccaaacatatcttcaaaaacacatattatgaggttgagggcggggttccatcaggttgctctggtactagcatatttaacaccatgatcaacaacataatcataaggactttggtgctggatgcttacaaaaatatagatttagataagttaaagatcttggcttatggggatgatgtaatcttttcctacaaatatcagcttgacatggaagcaattgctaaagaaggcgtcaagtatgggctgacaataacacctgctgacaaatccaatgattttaagcagcttaattatgacaatgtaacatttctcaaaagaggatttagacaggatgaaaagcatcaatttctgatacacccaacatttccaattgaagagatccaagaatccatacgatggacaaagaaaccatctcaaatgcaggaacatgtactgtcattgtgtcacttaatgtggcataatggcagagatgtgtacaagaagtttgaggagcgtatacgtagtgttagcgctggtcgtgcactatatattcctccttacgacttactactgcatgagtggtatgaaaaattttaaattgtttagatatagaaatattaaacatttagttaaaaaaaaaaaaaaaaaaaaaaaaagactctcgag

SEQ ID NO: 8 - DNA, 7206 bp, synthetic construct; identical to SEQ ID NO: 5
  source 1..7206: mol_type = other DNA

SEQ ID NO: 9 - DNA, 7304 bp, synthetic construct
  source 1..7304: mol_type = other DNA
taatacgactcactatagttaaaacagcggatgggtatcccaccattcgacccattgggtgtagtactctggtactatgtacctttgtacgcctgtttctccccaaccacccttccttaaaattcccacccatgaaacgttagaagcttgacattaaagtacaataggtggcgccatatccaatggtgtctatgtacaagcacttctgtttcccaggagcgaggtataggctgtacccactgccaaaagcctttaaccgttatccgccaaccaactacgtaacagttagtaccatcttgttcttgactggacgttcgatcaggtggattttccctccactagtttggtcgatgaggctaggaattccccacgggtgaccgtgtcctagcctgcgtggcggccaacccagcttatgctgggacgcccttttaaggacatggtgtgaagactcgcatgtgcttggttgtgagtcctccggcccctgaatgcggctaaccttaaccctagagccttatgccacgatccagtggttgtaaggtcgtaatgagcaattccgggacgggaccgactactttgggtgtccgtgtttctcatttttcttcatattgtcttatggtcacagcatatatatacatatactgtgatcatgggcgctcaggtttctacacagaaaagtggatctcacgaaaatcaaaacattttgaccaatggatcaaatcagactttcacagttataaattactataaggatgcagcaagtacatcatcagctggtcaatcactgtcaatggacccatctaagtttacagaaccagttaaagatctcatgcttaagggtgcaccagcattgaattcacccaatgttgaggcctgtggttatagtgatagagtacaacaaatcacactcgggaattcaacaataacaacacaagaagcagccaacgctgttgtgtgttatgctgaatggccagagtaccttccagatgtggacgctagtgatgtcaataaaacttcaaaaccagacacttctgtctgtaggttttacacattggatagtaagacatggacaacaggttctaaaggctggtgctggaaattaccagatgcactcaaggatatgggtgtgttcgggcaaaacatgtttttccactcactaggaagatcaggttacacagtacacgttcagtgcaatgccacaaaattccatagcggttgtctacttgtagttgtaataccagaacaccaactggcttcacatgagggtggcaatgtttcagttaaatacacattcacgcatccaggtgaacgtggtatagatttatcatctgcaaatgaagtgggagggcctgtcaaggatgtcatatacaatatgaatggtactttattaggaaatctgctcattttccctcaccagttcattaatctaagaaccaataatacagccacaatagtgataccatacataaactcagtacccattgattcaatgacacgtcacaacaatgtctcactgatggtcatccctattgcccctcttacagtaccaactggagcaactccctcactccctataacagtcacaatagcacctatgtgcactgagttctctgggataaggtccaagtcaattgtgccacaaggtttgccaactacaactttgccggggtcaggacaattcttgaccacagatgacaggcaatcccccagtgcactgccaaattatgagccaactccaagaatacacatactagggaaagttcataacttgctagaaattatacaggtagatacactcattcctatgaacaacacgcatacaaaagatgaggttaacagttacctcataccactaaatgcaaacaggcaaaatgagcaggtttttgggacaaacctgtttattggtgatggggtcttcaaaactactcttctgggtgaaattgttcagtactatacacattggtctggatcacttagattctcttcgatgtatactggtcctgccttgtccagtgctaaactcactctagcatacaccccgcctggtgctcgtggtccacaggacaggagagaagcaatgctaggtactcatgttgtctgggatattggtctgcaatccaccatagtaatgacaataccatggacatcaggggtgcagtttagatatactgatccagatacatacaccagtgctggctttctatcatgttggtatcaaacttctcttatacttcccccagaaacgaccggccaggtctacttattatcattcataagtgcatgtccagattttaagcttaggctgatgaaagatactcaaactatctcacagactgttgcactcactgaaggcttaggtgatgaattagaagaagtcatcgttgagaaaacgaaacagacggtggcctcaatctcatctggtccaaaacacacacaaaaagtccccatactaactgcaaacgaaacaggggccacaatgcctgttcttccatcagacagcatagaaaccagaactacctacatgcactttaatggttcagaaactgatgtagaatgctttttgggtcgtgcagcttgtgtgcatgtaactgaaatacaaaacaaagatgctactggaatagataatcacagagaagcaaaattgttcaatgattggaaaatcaacctgtccagccttgtccaacttagaaagaaactggaactcttcacttatgttaggtttgattctgagtataccatactggccactgcatctcaacctgattcagcaaactattcaagcaatttggtggtccaagccatgtatgttccacatggtgccccgaaatccaaaagagtgggcgattacacatggcaaagtgcttcaaaccccagtgtattcttcaaggtgggggatacatcaaggtttagtgtgccttatgtaggattggcatcagcatataattgtttttatgatggttactcacatgatgatgcagaaactcagtatggcataactgttctaaaccatatgggtagtatggcattcagaatagtaaatgaacatgatgaacacaaaactcttgtcaagatcagagtttatcacagggcaaagctcgttgaagcatggattccaagagcacccagagcactaccctacacatcaatagggcgcacaaattatcctaagaatacagaaccagtaattaagaagaggaaaggtgacattaaatcctatggtttaggacctaggtacggtgggatttatacatcaaatgttaaaataatgaattaccacttgatgacaccagaagaccaccataatctgatagcaccctatccaaatagagatttagcaatagtctcaacaggaggacatggtgcagaaacaataccacactgtaaccgtacatcaggtgtttactattccacatattacagaaagtattaccccataatttgcgaaaagcccaccaacatctggattgaaggaagcccttattacccaagtagatttcaagcaggagtgatgaaaggggttgggccggcagagctaggagactgcggtgggattttgagatgcatacatggtcccattggattgttaacagctgaaggtagtggatatgtttgttttgctgacatacgacagttggagtgtatcgcagaggaacaggggctgagtgattacatcacaggtttgggtagagcttttggtgtcgggttcactgaccaaatctcaacaaaagtcacagaactacaagaagtggcgaaagatttcctcaccacaaaagttttgtccaaagtggtcaaaatggtttcagctttagtgatcatttgcagaaatcatgatgacttggtcactgttacggccactctagcactacttggatgtgatggatctccttggagatttctgaagatgtacatttccaaacactttcaggtgccttacattgaaagacaagcaaatgatggatggttcagaaagtttaatgatgcatgtaatgctgcaaagggattggaatggattgctaataagatttccaaactgattgaatggataaaaaacaaagtacttccccaagccaaagaaaaactagaattttgtagtaaactcaaacaacttgatatactagagagacaaataaccaccatgcatatctcgaatccaacacaggaaaaacgagagcagttgttcaataacgtattgtggttggaacaaatgtcgcaaaagtttgccccattttatgccgttgaatcaaaaagaatcagggaactcaagaacaaaatggtaaattatatgcaatttaaaagtaaacaaagaactgaaccagtgtgtgtattaatccatggtacacccggttctggtaaatcattaacaacatccattgtgggacgtgcaattgcagaacacttcaattcagcagtatattcacttccaccagatcccaagcactttgatggttatcagcaacaggaagttgtgattatggatgatctgaaccaaaatccagatggacaggatataagcatgttttgtcaaatggtttcttcagtggatttcttgcctccaatggctagtttagataacaagggcatgttattcaccagtaattttgttctagcctccacaaattctaacacactaagccccccaacaatcttgaatcctgaagctttagtcaggagatttggttttgacctagatatatgtttgcatactacctacacaaagaatggaaaactcaatgcaggcatgtcaaccaagacatgcaaagattgccatcaaccatctaatttcaagaaatgttgccccctagtctgtggaaaagctattagcttggtagacagaactaccaacgttaggtatagtgtggatcaactggtcacggctattataagtgatttcaagagcaaaatgcaaattacagattccctagaaacactgtttcaaggaccagtgtataaagatttagagattgatgtttgcaacacaccaccttcagaatgtatcaacgatttactgaaatctgtagattcagaagagattagggaatattgtaagaagaagaaatggattatacctgaaattcctaccaacatagaaagggctatgaatcaagccagcatgattattaatactattctgatgtttgtcagtacattaggtattgtttatgtcatttataaattgtttgctcaaactcaaggaccatattctggtaacccgcctcacaataaactaaaagccccaactttacgcccagttgttgtgcaaggaccaaacacagaatttgcactatccctgttaaggaaaaacataatgactataacaacctcaaagggagagttcacagggttaggcatacatgatcgtgtctgtgtgatacccacacacgcacagcctggtgatgatgtactagtgaatggtcagaaaattagagttaaggataagtacaaattagtagatccagagaacattaatctagagcttacagtgttgactttagatagaaatgaaaaattcagagatatcaggggatttatatcagaagatctagaaggtgtggatgccactttggtagtacattcaaataactttaccaacactatcttagaagttggccctgtaacaatggcaggacttattaatttgagtagcacccccactaacagaatgattcgttatgattatgcaacaaaaactgggcagtgtggaggtgtgctgtgtgctactggtaagatctttggtattcatgttggcggtaatggaagacaaggattttcagctcaacttaaaaaacaatattttgtagagaaacaaggccaagtaatagctagacataaggttagggagtttaacataaatccagtcaacacggcaactaagtcaaaattacatcccagtgtattttatgatgtttttccaggtgacaaggaacctgctgtattgagtgacaatgatcccagactggaagttaaattgactgaatcattattctctaagtacaaggggaatgtaaatacggaacccactgaaaatatgcttgtggctgtagaccattatgcagggcaactattatcactagatatccccacttctgaacttacactaaaagaagcattatatggagtagatggactagaacctatagatattacaaccagtgcaggatttccctatgtgagtcttgggatcaaaaagagagacattctgaataaagagacccaggacacagaaaagatgaagttttatctagacaagtatggcattgacttgcctctagttacatatattaaggatgaattaagaagtgttgacaaagtccgattagggaaaagtagattaattgaagcctccagtttgaatgattctgttaacatgagaatgaaactaggcaacctttacaaagcattccatcaaaatcccggtgttctgactggatcagcagtgggttgtgatcctgatgtgttttggtctgtcatcccttgcttaatggatgggcacctgatggcatttgattactctaattttgatgcctctttgtcaccagtttggtttgtctgtctagagaaggttttgaccaagttaggctttgcaggctgtgcattaattcaatcaatttgtaatacccatcatatctttagggatgaaatatatgtggttgaaggtggcatgccctcagggtgttcaggaaccagcatattcaattccatgatcaacaacataatcattaggactttgatattagatgcatataaaggaatagatttagacaaacttaaaatcttagcttacggtgatgatttgattgtttcttatccttatgaactggatccacaagtgttggcaactcttggtaaaaattatggactaaccatcacacccccagacaaatctgaaacttttacaaaaatgacatgggaaaacttgacatttttaaagagatacttcaagcctgatcaacaatttccctttttggttcacccagttatgcccatgaaagatatacatgagtcaatcagatggacaaaggatcctaaaaacacacaggatcacgtccgatcattatgcatgttagcatggcactcaggagaaaaagagtacaatgaattcattcagaagatcagaactactgacattggaaaatgtctaattctcccagaatacagcgtacttaggaggcgctggttggacctcttttaggttaacaatatagacacttaatttgagtagaagtaggagtttataaaaaaaaaaaaaaaaaaaaaaaaaaaaaaaaaaaaaaaaaaaaaaaaaagaagagctttagtgagggttaatt

SEQ ID NO: 10 - DNA, 7202 bp, synthetic construct
  source 1..7202: mol_type = other DNA
gcggccgcatttaggtgacactatagttaaaactgggtttgggttgttcccacccaaaccacccacgcggtgttgtacactgttattccggtaaccttgtacgccagttttatatcccttcccccccttgtaacttagaagacatgcgaatcgaccaatagcaggcaatcaaccagattgtcaccggtcaagcacttctgtttccccggctctcgttgatatgctccaacagggcaaaaacaattggtagtcgttacccgcaagatgcctacgcaaaacctagtagcatcttcgaagatttttggttggtcgctcagttgctaccccagcaatagacctggcagatgaggctagaaataccccactggtgacagtgttctagcctgcgtggctgcctgcacacccacacgggtgtgaagccaaagattggacaaggtgtgaagagcacgtgtgctcatcttgaagtcctccggcccctgaatgcggctaaccttaaccccgtagccattgctcgcaatccagcgagtatatggtcgtaatgagtaattacgggatgggaccgactactttgggtgtccgtgtttcactttttacttatcaatttgcttatggtgacaatatatatagatatatattgacaccatgggcgcccaagtatctagacagaatgttggtacgcactctacacaaaactctgttacaaatggatcaagtctaaattacttcaacatcaactacttcaaggatgcggcatcaagtggagcttccaaattagaattttcacaagaccccagcaaattcacagacccagtgaaagatgttctggagaaaggaattcccaccctacaatctcccacagttgaagcgtgtggttattcagacaggataatccaaatcactagaggtgattctacgattacttcacaggatgtagctaatgcagttgttgcatatggagtttggcctcattatctcacaccacaagatgcaactgctatagataaaccaactagacctgatacttcctccaatagattttatactttagaaagccagatgtggtcccctgaatccaaaggatggtggtggaagttacctgatgctttgaaggacatgggcatttttggggaaaacatgttttaccatttccttgggagaagtggttatacagtccatgttcaatgtaatgccagtaaatttcatcaaggaacactattagttgccatgataccagaacatcagttggctgccgccaagggtggcactgtcacagcaggatataaatacacgcatcctggtgaagcgggcagggatgttgggaaaactgaccgcaagcaaaacaaccgacagcctagtgatgacaattggctcaatttcgatggtactctcttaggcaactcgaccatcttcccacaccaatttatcaatctcaggagtaataattcagctactattatagttccatatgttaatgcagttccaatggattctatgcttagacacaataattggagtttggtgattatacctatatgcaaacttaggtgtcaaggtatatccccagttgtacctatcacaatatctataagccctatgtgtgctgagttttctggagctagagctagaaatgtcactcaaggactgccaacatttataacacctggatctggtcaattcttaactacagatgatttccaatctcccagtgcattgccgtggtatcaccctacaaaagaaatatcaataccaggacaagtccgaaacctagttgaactatgtcaagttgatactatgataccaatcaacaatattgcagcaaatgtcaccaatgagagcatgtatgccattagtcttgagagcaatgctgatattaaacaagctgtgtttgcaatgagagttgacatcacctcacaacccttagctacaacattaataggtgagatagctagttattacactcattggacagggagccttcgatttagtttcatgttttgtggttctgcattttcaactcttaaattgctacttgcttatacaccaccagggattgctgttccagatagcagaaagaaagctatgcttggtacacatgtcatatgggatgttggtttgcaatcaacagtatcaatagtggtaccttgggtgagtgctagccattacagaaatacaacccctgacacttattcattagctggttatatcacatgctggtatcaaacaaaactagtggaacccccaaacacaaccccagtggcagacatgatatgctttgtttcagggtgtaaggatttttgtttacgcatggcaagagatacaaatctacataaacaaagtggtgtgattgcacaaaatcctgtagaaagatatgtagatgaagttttgaatgaagtccttgtagtacccaatatcaatgaaagtaatccgactacatccaactcagcacctgcactggacgctgcagaaactggccataccagtggtgtacaaccagaagacatgattgagacccgttatgtccaaacatcacagactagagatgaaatgagcattgaaagcttccttggcaggtcaggttgtatacacatgtcaactatgaatataaattatgaaaattatgatgatgctcctgaaaattttaccaaatggaaaataagtttacaagagatggctcaaatacgtaggaaatttgaattattcacctatgtaagatttgattcagaagtgacaattgtaccatgtatagctggtcaaagtggagatgtgggacatgtagttatgcagtatatgtatgtcccacctggagcccctctacccacaaaaagaaatgactacacatggcagtccggcactaatgcatcagtattctggcaacatggtcaaacttaccccagattctcattaccattccttagtatagcatccgcatattatatgttttatgatggatatgatggtgattccgcacaatcacattatggcaccacagtagttaatgacatgggcacattatgctttaggatagtgactgaagaacacactagcagggtaaaggttactactagaatctatcataaggctaaacatgttaaagcttggtgtccaagacctcctagggcagtagaatatacaaatgcacatgtgaccaattataaacccactgatggagaagttactactgccattaggcatagagataatgttagagccatccaaaattttggacctagtgacatgtatgtgcatgttggaaacctaatatacagaaatctacacttgttcaactcagagatgcatgattcaatcttggtatcctactcatctgatctagtcatttaccgaacaaacactataggtgatgactttatcccatcttgtgattgcactgaagctacatattactgtaaacacaaaaatagatattatccaatcaaagtcactagtcatgattggtatgaaatacaagagagtgagtattatccaaaacacatacagtacaatttgctaattggcgaaggtccctgtgaacctggagattgtggaggtaaactactatgtaaacatggggtgattgggatcattacagctggtggtgagggccatgttgctttcattgatttgagacatttcttatgtgctgaggaacaaggtgttactgactatatacatatgttaggtgaagcttttggcaatggttttgtagatagtgttaaagaacatgttaatgcaataaaccctattaatagtattagtaaaaaagtgattaaatggctactgaggattgtatccgctatggtcattatcattagaaattcttcagatccacatactgtcattgctactctaacattaattggttgttctgggtcgccttggaggttcttgaaagagaaattttgcaaatggacccagttaaattatatccataaagaatctgattcatggctaaagaaatttactgaaatgtgtaatgcagcgagaggtcttgagtggataggcaataagatatctaagtttatagaatggatgaaatcaatgttaccacaagcacaactcaaagttaagtatttgaatgagttaaagaaattgagcttacttgaaaaacaggttgaaaacttaagatgtgccgacactaagacacaagagaagataaaagttgagatagacaccctgcatgatttatcttgtaaatttctcccattatatgccagtgaggctaaaagaatcaaagttatttataacaaatgcaataatatcatcaagcaaaagaaaagaagtgaacctgttgcagtaatgatacatggttcacctgggactggcaaatctatcacaacaagctttctggcaagaatgattactaatgaaagtgacatatactctttacctcctgacccaaagtactttgatggctatgatcagcaaagtgtcgttataatggatgacattatgcaaaatcctgacggtgaggacatgtcactgttttgccaaatggtttccagtgtgaccttcattccaccaatggctgatttgccagacaaggggaaaccctttgattcaagatttgttctatgtagcactaaccactctctcttagctcctcccaccatcacctctcttcaggcaatgaataggcggttcttcttagacttagatattgtggtccatgataattacaaagatacacaaggcaagttggatgtgtccaaagcattcaaaccatgtgatgttgggacaaaaataggaaatgcccgctgttgcccatttatatgtggcaaagcagtaaccttcaaagaccgcaatacttgcctgagttattcattgagccaaatttacaacttgattcttcaggaagacaaaaggcgcacccatgttgtggacgtcatgactgctatatttcaaggaccaatttcaatggaagtccccccccctcctgctataactgatctcttacgctctgtcagaacaccagaagttattaaatactgtgaaaacaacaaatggatcgttccagctgactgcaaaattgagcgagatttgaaccttgcaaataatatcataacaatcattgctaatataattagtatagcaggcatcatatacattatatacaaactgttctgctcttttcaaggaccctattcaggtgaacccaaaccaaaaacaaaagtcccagagaggagggtggttgcacaaggtccggaggaagaatttggccgctctctaatcaaacacaacacatgtgtagtcactactgataatggtaaatttactgggctgggaatctatgacaaacttatgatattacccacacatgctgatcctggtaaggaggtatatatcaatgggattgcaaccaaagttagtgattcatatgacttgtataataaacagggtgttaaattagagattacagctatagtgctagatagaaatgaaaagttcagggatatacgcaagtatataccggaaagggaggatgattaccctgattgcaacttagcccttgtagccaatcaatctgaacccactattataaatgtaggggatgtgatctcctatggtaacatcttgctcagtggcaatcaaacggcacgaatgcttaaatataattatcccacaaaatcagggtattgtggtggtgttctatataaaataggccaaattataggaatccatgtgggtggtaatggcagggatggattttctgcaatgttactcagatcttacttcaatcaaactcaaggagaaataacagtgacaaagaaggtggctgaatgtggacttccaacgatacacactccgtccaaaactaagctacaacctagtgtcttctttgatgtttttgaaggatccaaagaaccagctgtcctaactgacaaagacccccgattaactactgattttaataaagctctattttccaaatacaagggtaatatagaatgtagcatgactgatcatatgagggttgctatctcacactattcagctcaattaatgaccctggacattgattcaacaaatatgtccctagaggagagtgtatttggcactgaaggcttggaggctcttgatctaaatacaagtgcaggctttccatatataagtatgggaattaggaaaaaagatctaattaataacagtacaaaggacataactaaattgaagctagcacttgataaatatggggttgatttaccaatggttacattcctcaaagatgaacttagaaagaaggagaaaatatccactggcaaaacaagagtgattgaagctagtagtgtgaatgatacaatagcttttagggtagtgtatggaaatttgttttctacttttcacaagaaccctggtgtagtcacaggatctgcggtgggatgtgaccctgagaccttttggtcaaagataccagtgatgctagatggggaatgtataatggcatttgattacaccaattatgatggtagtatacacccaatttggtttgaagctctaaaacaagttctaaacaatttatcatttgaagacaagcttatagatagattgtgtagatcaaaacatatatttagagacacatactatgaagttgaaggtggggtgccctcaggttgttcaggaactagcatatttaatactatgataaataacatcattattaggactttagttcttgatgcttataagcatattgacttggacaaacttaaaataatagcttatggtgatgatgttattttctcttataagtatcccttagacatggaagcaattgctgtagaaggaaataagtacggtttgaccattacaccggctgataaatcagatacttttaggaagcttgattatgacaatgtaactttcctgaaaagaggttttaggcaagatgctaaatatccattcctaattcatcctacatttcctgtttctgagattcatgaatcaattagatggaccaagaaaccctcacagatgcaggaacatgttttatccttgtgtcatttaatgtggcacaatggacgggatgtgtacaaggagtttgagaggaagatacgcagcgtgagcgctggacgcgcgctgtacatccccccttatgagctactcctgcatgagtggtatgaaaaattttaaattatatatagaaataataaacatttagttttttagttttacaaaaaaaaaaaaaaaaaaaaaaaaagactctcgag